(19) United States
(12) Patent Application Publication
(10) Pub. No.: US 2010/0010058 A1
(43) Pub. Date: Jan. 14, 2010

(54) Azoline Compounds for Combating Arthropod Pests

(75) Inventors: Christopher Koradin, Ludwigshafen (DE); Markus Kordes, Bobenheim-Roxheim (DE); Ronan Le Vezouet, Mannheim (DE); Deborah L. Culbertson, Fuquay Varina, NC (US); Douglas D. Anspaugh, Apex, NC (US); Henry Van Tuyl Cotter, Raleigh, NC (US)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/305,267

(22) PCT Filed: May 25, 2007
(86) PCT No.: PCT/EP0207/055133

§ 371 Date: Dec. 17, 2008

Related U.S. Application Data
(60) Provisional application No. 60/815,743, filed on Jun. 22, 2006.

(51) Int. Cl. A01N 43/78; C07D 277/18; A01P 5/00; A01P 7/00

(52) U.S. Cl. 514/370; 548/198; 548/193

(57) ABSTRACT

The present invention relates to azoline compounds and their salts which are useful for combating arthropod pests. The present invention also relates to a method for combating arthropod pests and to agricultural compositions for combating said pests. It has been found that these objectives can be achieved by azoline compounds of the general formulae Ia or Ib, wherein X is S, O or NR⁴; Ar is phenyl or a 5 or 6 membered heteroaromatic ring; R¹ is H, C₁-C₆-alkyl, C₂-C₆-alkenyl, phenyl, a heteroaromatic ring etc.; R^{2a}, R^{2b} are H, CN, C₁-C₆-alkyl etc.; R¹ together with R^{2a} may also form a linear C₂-C₄-alkandiyl; R^{3a-d} are H, halogen, C₁-C₆-alkyl etc.

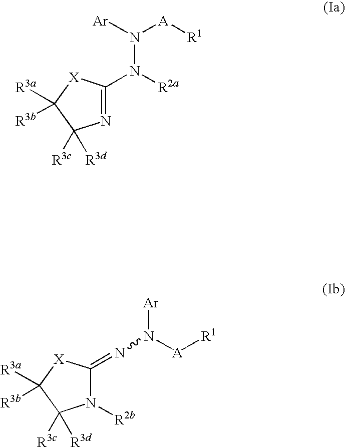

The present invention relates to azoline compounds, in particular to (azolin-2-yl)hydrazino compounds, (3H-azolin-2-yliden)hydrazino compounds, and their salts. The compounds are useful for combating arthropod pests and plant nematodes. The present invention also relates to a method for combating arthropod pests or nematodes and to agricultural compositions for combating said pests.

Arthropod pests and in particular insects and acarids and also plant-parasitic nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new compounds which provide effective control of such pests.

WO 2005 06 37 24 describes 1-(azolin-2-yl)-amino-1,2-diphenylethane compounds for combating insects, arachnids and nematodes.

DE 31 339 18 describes 2-arylhydrazino-2-thiazoline compounds of the general formula

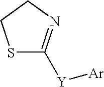

wherein Y is —N═N— or —NR¹—NR²—, with Ar being disubstituted phenyl, R¹ and R² being hydrogen or an acyl substituent and their use for fighting animal ecto- and endoparasites, especially ticks and gastric and intestinal nematodes. However, these compounds are limited in their activity or with regard to breadth of their activity spectrum.

For these reasons, there is still need for further compounds which have good pesticidal activity in particular against plant-parasitic pest. These compounds should also show a broad activity spectrum against a large number of different arthropod pests, especially against difficult to control insects, acarids and nematodes. Therefore, it is an object of the present invention to provide new compounds having good pesticidal activity and show a broad activity spectrum.

It has been found that these objectives can be achieved by azoline compounds of the general formulae Ia or Ib,

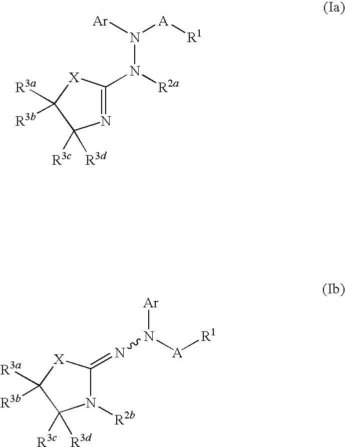

wherein
X is sulfur, oxygen or a radical NR⁴;
Ar is an aromatic radical selected from the group consisting of phenyl and a 5 or 6 membered heteroaromatic ring, which contains 1, 2, 3 or 4 heteroatoms selected from oxygen, sulfur and nitrogen as ring members, wherein Ar is unsubstituted or may carry any combination of 1, 2, 3, 4 or 5 radicals R^{y1};
R¹ is selected from the group consisting of hydrogen, CN, SH, OH, C₁-C₆-alkyl, which may carry 1, 2 or 3 radicals R^{a1}, C₃-C₁₀-cycloalkyl, which may be unsubstituted or may carry 1, 2, 3, 4 or 5 radicals R^{b1}, C₂-C₆-alkenyl, which may carry 1, 2 or 3 radicals R^{c1}, C₂-C₆-alkynyl, which may carry 1, 2 or 3 radicals R^{c1}, a radical O—R^{z1}, a radical S(O)ₘ—R^{z2} with m being 0, 1 or 2, a radical C(O)—R^{z3}, a radical NR^{z4}R^{z5},
   phenyl, which is unsubstituted or may carry any combination of 1, 2, 3, 4 or 5 radicals R^{y2},
   and a 5 or 6 membered heteroaromatic ring, which contains 1, 2, 3 or 4 heteroatoms selected from oxygen, sulfur and nitrogen as ring members, and which is unsubstituted or may carry any combination of 1, 2, 3, or 4 radicals R^{y3};
R^{2a}, R^{2b} are each independently selected from the group consisting of hydrogen, formyl, CN, C₁-C₆-alkyl, C₃-C₆-alkenyl, C₃-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, C₁-C₆-alkylsulfonyl,
   wherein the carbon atoms in the aliphatic radicals of the aforementioned groups may carry any combination of 1, 2 or 3 radicals R^{a2},
   C(O)NR^{a}R^{b}, C(S)NR^{a}R^{b}, (SO₂)NR^{a}R^{b},
   phenyl, benzyl, phenoxycarbonyl, phenylsulfonyl, 5 or 6 membered hetarylmethyl, 5 or 6 membered hetarylcarbonyl and benzoyl each of the last seven mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from oxygen, sulfur and nitrogen as ring members;
R¹ together with R^{2a} may also form a linear C₂-C₄-alkandiyl, which may carry 1, 2, 3 or 4 radicals R^{d} independently selected from halogen, C₁-C₄-alkyl and C₁-C₄-haloalkyl, wherein the radicals which are bound to adjacent carbon atoms of alkylene may together with the carbon atoms to which they are bound, form a fused phenyl ring, which may carry 1, 2, 3 or 4 radicals independently selected from halogen, C₁-C₄-alkyl and C₁-C₄-haloalkyl, and wherein 1 or 2 non-adjacent CH₂-moieties of C₂-C₄-alkandiyl may be replaced by oxygen or a radical N—R^{q}; wherein R^{q} has one of the meanings given for R^{2a};
R^{3a}, R^{3b}, R^{3c}, R^{3d} are each independently selected from the group consisting of hydrogen, halogen, cyano, nitro, hydroxy, mercapto, amino, C₁-C₆-haloalkyl, C₁-C₆-alkyl, C₁-C₆-alkylamino, Di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
   C₃-C₆-cycloalkyl, phenyl or benzyl, each of the last two mentioned radicals may be unsubstituted or may carry any combination of 1, 2, 3, 4 or 5 radicals R^{b3};
R⁴ is selected from the group consisting of hydrogen, formyl, CN, C₁-C₆-alkyl, C₃-C₆-alkenyl, C₃-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a4},
   C(O)NR^{a}R^{b}, (SO₂)NR^{a}R^{b},
   phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b4}, and wherein the 5 or 6 membered heteroaromatic ring in hetarylmethyl and hetarylcarbonyl contains 1, 2, 3 or 4 heteroatoms selected from oxygen, sulfur and nitrogen as ring members;
A is linear C₁-C₄-alkandiyl, which is unsubstituted or may carry any combination of 1, 2, 3 or 4 radicals R^{x}; or A can also be C(O) when R¹ is a radical O—R^{z1} or NR^{z4}R^{z5};
R^{x} is selected from the group consisting of halogen, C₁-C₆-alkyl and C₁-C₆-haloalkyl, wherein the carbon atoms in these groups may carry any combination of 1, 2 or 3 radicals Rax;
R^{y1}, R^{y2}, R^{y3} independently of each other are selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{c}R^{d}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals Ray,
   C(O)NR^{a}R^{b}, (SO₂)NR^{a}R^{b}, and radicals of the formula Y-Cy, wherein
   Y is a single bond, oxygen, sulfur or C₁-C₆-alkandiyl, wherein one carbon might be replaced with oxygen.
   Cy is selected from the group consisting of C₃-C₁₂-cycloalkyl, which is unsubstituted or substituted with any combination of 1, 2, 3, 4 or 5 radicals R^{bx}, phenyl, naphthyl and mono- or bicyclic 5- to 10-membered heterocyclyl, which contains 1, 2, 3 or 4 heteroatoms selected from oxygen, sulfur and nitrogen as ring members, wherein Cy is unsubstituted or may carry any combination of 1, 2, 3, 4 or 5 radicals R^{by};
   and wherein two radicals R^{y1}, R^{y2} or R^{y3} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from oxygen, sulfur and nitrogen as ring members, and wherein the fused ring is unsubstituted or may carry any combination of 1, 2, 3, or 4 radicals R^{by};
R^{z1} is selected from the group consisiting of C₁-C₆-alkyl, C₁-C₆-haloalkyl, C₁-C₆-alkoxy-C₁-C₄-alkyl, C₁-C₆-alkylcarbonyl, C₁-C₆-haloalkylcarbonyl, C₂-C₆-alkenylcarbonyl, C₂-C₆-alkynylcarbonyl, phenyl, benzyl and benzoyl, each of the last three mentioned radicals may be unsubstituted or may carry any combination of 1, 2, 3, 4 or 5 radicals R^{bz};
R^{z2} is selected from the group consisiting of C₁-C₆-alkyl, C₁-C₆-haloalkyl, and phenyl, which may be unsubstituted or may carry any combination of 1, 2, 3, 4 or 5 radicals R^{bz};
R^{z3} is selected from the group consisiting of hydrogen, C₁-C₆-alkyl, C₁-C₆-haloalkyl, C₁-C₆-alkoxy-C₁-C₄-alkyl, C₁-C₆-alkoxy, C₁-C₆-haloalkoxy, C₂-C₆-alkenyl, C₂-C₆-alkynyl, C₂-C₆-alkenyloxy, C₂-C₆-alkynyloxy, a radical NR^{c}R^{d}, phenyl, benzyl and phenoxy, each of the last three mentioned radicals may be unsubstituted or may carry any combination of 1, 2, 3, 4 or 5 radicals R^{bz};
R^{z4}, R^{z5} are each independently selected from the group consisting of hydrogen, C₁-C₆-alkyl, C₂-C₆-alkenyl, C₂-C₆-alkynyl, C₂-C₆-alkylcarbonyl, C₂-C₆-alkoxycarbonyl, wherein the carbon atoms in these groups may carry any combination of 1, 2 or 3 radicals R^{az}, or phenyl, phenyl-C₁-C₄-alkyl, benzoyl, wherein the last three mentioned groups may carry any combination of 1, 2, 3, 4 or 5 radicals R^{bz}
R^{a}, R^{b} are each independently selected from the group consisting of hydrogen, C₁-C₆-alkyl, C₂-C₆-alkenyl, or C₂-C₆-alkynyl, wherein the carbon atoms in these groups may carry any combination of 1, 2 or 3 radicals R^{ay};
R^{a1}, R^{a2}, R^{a3}, R^{a4}, R^{ax}, R^{ay} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{bx}, R^{by} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R^{c}, R^{d} are each independently selected from the group consisting of hydrogen, C₁-C₆-alkyl, C₂-C₆-alkenyl, or C₂-C₆-alkynyl, wherein the carbon atoms in these groups may carry any combination of 1, 2 or 3 radicals R^{ay};
R^{c1} is selected from the group consisting of halogen, OH, C₁-C₆-alkoxy and C₃-C₆-cycloalkyl;
   and by salts of the compounds of formulae Ia and Ib.

Therefore, the present invention relates to the aforementioned azoline compounds, i.e. (azolin-2-yl)hydrazino compounds of the formula Ia and their related (3H-azolin-2-yliden)hydrazino compounds of the general formula Ib and to the salts thereof. These compounds have a high pesticidal activity and are active against a broad spectrum of arthropod pests, in particular insects or acarids, and also against nematodes, in particular plant-parasitic nematodes.

Therefore, the present invention also relates to the use of compounds of formulae Ia or Ib and their salts for combating arthropod pests, in particular insects or acarids, or nematodes, in particular plant-parasitic nematodes.

Furthermore, the present invention relates to compositions, in particular agricultural compositions, comprising at least one compound of formulae Ia or Ib and/or a salt, in particular an agriculturally acceptable salt, thereof and a carrier material.

Furthermore, the invention relates to a method for the control of arthropod pests and nematodes, which comprises contacting said pest, their habit, breeding ground, food supply, plant, seed, soil, area, material or environment in which the pest is growing or may grow, or the materials, plants, seeds, soils, surfaces or spaces to be protected from an attack or infestation by said pests with a pesticidally effective amount of at least one (azolin-2-yl)hydrazino compound of the general formula Ia and/or at least one (3H-azolin-2-yliden)hydrazino compound of the general formula Ib and/or at least one salt thereof, in particular an agriculturally acceptable salt.

Furthermore, the present invention provides a method for protecting growing plants attack or infestation by insects, acarids or nematodes, which comprises applying to the plants, or to the soil or water in which they are growing, at least one compound of the general formulae Ia or Ib and/or at least one agriculturally acceptable salt thereof.

Furthermore, the invention relates to a method of protection of seed comprising contacting the seeds with at least one compound of formulae Ia or Ib and/or an agriculturally acceptable salt thereof or a composition containing at least one of these compounds in pesticidally effective amounts.

Furthermore, the invention relates to seed, comprising at least one compound of formulae Ia or Ib and/or an agriculturally acceptable salt thereof as defined above in an amount of from 0.1 g to 10 kg per 100 kg of seeds, calculated as the compound of formulae Ia or Ib.

Furthermore, the invention relates to a method for treating, controlling, preventing or protecting animals against infestation or infection by parasites which comprises administering or applying to the animals a parasiticidally effective amount of at least one compound of formulae Ia or Ib and/or an veterinarily acceptable salt thereof.

The compounds of the general formulae Ia and Ib may have one or more centers of chirality, in which case they are present as mixtures of enantiomers or diastereomers.

The present invention provides both the pure enantiomers or diastereomers or mixtures thereof. The compounds of the general formulae Ia or Ib may also exist in the form of different tautomers. The invention comprises the single tautomers, if separable, as well as the tautomer mixtures.

Salts of the compounds of the formulae Ia and Ib are especially agriculturally acceptable salts and veterinarily acceptable salts. They can be formed in a customary method, e.g. by reacting the compound with an acid of the anion in question if the compound of formula Ia or Ib has a basic functionality or by reacting an acidic compound of formula Ia or Ib with a suitable base.

Suitable agriculturally useful and veterinarily acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae Ia and Ib with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

Examples of other meanings are:

The term “C₁-C₆-alkyl” as used herein and in the alkyl moieties of C₁-C₆-alkoxy, C₁-C₆-alkylamino, di(C₁-C₆-alkyl)amino, C₁-C₆-alkylthio, C₁-C₆-alkylsulfonyl, C₁-C₆-alkylsulfoxyl, C₁-C₆-alkylcarbonyl, C₁-C₆-alkoxycarbonyl, C₁-C₆-alkylthiocarbonyl, and C₁-C₆-alkylcarbonyloxy refer to a saturated straight-chain or branched hydrocarbon group having 1 to 6 carbon atoms, especially 1 to 4 carbon groups, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl,

3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and their isomers. C₁-C₄-alkyl means for example methyl, ethyl, propyl, 1-methylethyl, butyl, 1-methylpropyl, 2-methylpropyl or 1,1-dimethylethyl.

In each alkyl radical the carbon atoms may carry 1, 2 or 3 radicals R^{#}. In other words, each of the hydrogen atoms in these radicals may independently from the others be replaced by one of the aforementioned radicals R^{#}. In case of R^{#} being halogen usually 1, 2, 3 or all of the hydrogen atoms in said alkyl radical are replaced by halogen, especially by fluorine or chlorine. These radicals are also referred to as haloalkyl. In case of R^{#} being cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, or C₁-C₆-alkylthio usually 1 or 2 of the hydrogen atoms in said alkyl radical may be replaced by the radical R^{#}.

The term “C₁-C₆-haloalkyl” as used herein and in the haloalkyl moieties of C₁-C₆-alkoxy and haloalkylthio refers to a straight-chain or branched saturated alkyl group having 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “C₁-C₆-alkoxy” as used herein refers to a straight-chain or branched saturated alkyl group having 1 to 6 carbon atoms (as mentioned above) which is attached via an oxygen atom. Examples include C₁-C₆-alkoxy such as methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethyl propoxy, n-hexoxy, 1-methyl pentoxy, 2-methyl pentoxy, 3-methyl pentoxy, 4-methyl pentoxy, 1,1-dimethylbutoxy, 1,2-dimethylbutoxy, 1,3-dimethylbutoxy, 2,2-dimethylbutoxy, 2,3-dimethyl butoxy, 3,3-dimethylbutoxy, 1-ethyl butoxy, 2-ethyl butoxy, 1,1,2-trimethylpropoxy, 1,2,2-trimethylpropoxy, 1-ethyl-1-methylpropoxy, 1-ethyl-2-methylpropoxy and the like.

The term “C₁-C₆-haloalkoxy” as used herein refers to a C₁-C₆-alkoxy group as mentioned above wherein the hydrogen atoms are partially or fully substituted by fluorine, chlorine, bromine and/or iodine, i.e., for example, C₁-C₆-haloalkoxy such as chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy, 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluorohexoxy, in particular chloromethoxy, fluoromethoxy, difluoromethoxy, trifluoromethoxy, 2-fluoroethoxy, 2-chloroethoxy or 2,2,2-trifluoroethoxy.

The term “C₁-C₆-alkoxy-C₁-C₆-alkyl” as used herein refers to C₁-C₆-alkyl wherein 1 carbon atom carries a C₁-C₆-alkoxy radical as mentioned above. Examples are CH₂—OCH₃, CH₂—OC2H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl, 4-(1,1-dimethylethoxy)butyl and the like.

The term “C₁-C₆-alkylcarbonyl” as used herein refers to a straight-chain or branched saturated alkyl group having 1 to 6 carbon atoms (as mentioned above) bonded via the carbon atom of the carbonyl group at any bond in the alkyl group. Examples include C₁-C₆-alkylcarbonyl such C(O)—CH₃, C(O)—C₂H₅, n-propylcarbonyl, 1-methylethylcarbonyl, n-butylcarbonyl, 1-methylpropylcarbonyl, 2-methylpropylcarbonyl, 1,1-dimethylethylcarbonyl, n-pentylcarbonyl, 1-methylbutylcarbonyl, 2-methylbutylcarbonyl, 3-methylbutylcarbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl and the like.

The term “C₁-C₆-alkoxycarbonyl” as used herein refers to a straight-chain or branched alkoxy group (as mentioned above) having 1 to 6 carbon atoms attached via the carbon atom of the carbonyl group. Examples include (C₁-C₆-alkoxy)carbonyl, for example CO—OCH₃, CO—OC2H₅, CO—O—CH₂—C₂H₅, CO—OCH(CH₃)₂, n-butoxycarbonyl, CO—OCH(CH₃)—C₂H₅, CO—OCH₂CH(CH₃)₂, CO—OC(CH₃)₃, n-pentoxycarbonyl, 1-methyl butoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethyl butoxycarbonyl, 1,2-dimethyl butoxycarbonyl, 1,3-dimethylbutoxycarbonyl, 2,2-dimethyl butoxycarbonyl, 2,3-dimethyl butoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethyl propoxycarbonyl, 1-ethyl-1-methylpropoxycarbonyl or 1-ethyl-2-methylpropoxycarbonyl

The term “C₁-C₆-alkylcarbonyloxy” as used herein refers to a straight-chain or branched saturated alkyl group having 1 to 6 carbon atoms (as mentioned above) bonded via the carbon atom of the carbonyloxy group at any bond in the alkyl group. Examples include C₁-C₆-alkylcarbonyloxy such O—C(O)—CH₃, O—C(O)—C₂H₅, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy, 1,2-dimethylpropylcarbonyloxy and the like.

The term “C₁-C₆-alkylthio” (C₁-C₆-alkylsulfanyl: C₁-C₆-alkyl-S—) as used herein refers to a straight-chain or branched saturated alkyl group having 1 to 6 carbon atoms (as mentioned above) which is attached via a sulfur atom, for example C₁-C₆-alkylthio such as methylthio, ethylthio, propylthio, 1-methylethylthio, butylthio, 1-methylpropylthio, 2-methylpropylthio, 1,1-dimethylethylthio, n-pentylthiocarbonyl, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methyl pentylthio, 1,1-dimethylbutylthio, 1,2-dimethylbutylthio, 1,3-dimethylbutylhio, 2,2-dimethylbutylthio, 2,3-dimethylbutylthio, 3,3-dimethylbutylthio, 1-ethylbutlthio, 2-ethylbutylthio, 1,1,2-trimethylpropylthio, 1,2,2-trimethylpropylthio, 1-ethyl-1-methylpropylthio or 1-ethyl-2-methylpropylthio.

The term “C₁-C₆-alkylthiocarbonyl” as used herein refers to a straight-chain or branched alkthio group (as mentioned above) having 1 to 6 carbon atoms attached via the carbon atom of the carbonyl group. Examples include C(O)—SCH₃, C(O)—SC2H₅, C(O)—SCH₂—C₂H₅, C(O)—SCH(CH₃)₂, n-butylthiocarbonyl, C(O)—SCH(CH₃)—C₂H₅, C(O)—SCH₂—CH(CH₃)₂, C(O)—SC(CH₃)₃, n-pentylthiocarbonyl, 1-methylbutylthiocarbonyl, 2-methylbutylthiocarbonyl, 3-methylbutylthiocarbonyl, 2,2-dimethylpropylthiocarbonyl, 1-ethylpropylthiocarbonyl, n-hexylthiocarbonyl, 1,1-dimethylpropylthiocarbonyl, 1,2-dimethylpropylthiocarbonyl, 1-methylpentylthiocarbonyl, 2-methylpentylthiocarbonyl, 3-methylpentylthiocarbonyl, 4-methylpentylthiocarbonyl, 1,1-dimethylbutylthiocarbonyl, 1,2-dimethylbutylthiocarbonyl, 1,3-dimethylbutylhiocarbonyl, 2,2-dimethylbutylthiocarbonyl, 2,3-dimethylbutylthiocarbonyl, 3,3-dimethylbutylthiocarbonyl, 1-ethylbutlthioycarbonyl, 2-ethylbutylthiocarbonyl, 1,1,2-trimethylpropylthiocarbonyl, 1,2,2-trimethylpropylthiocarbonyl, 1-ethyl-1-methylpropylthiocarbonyl or 1-ethyl-2-methylpropylthiocarbonyl

The term “C₁-C₆-alkylsulfoxyl” (C₁-C₆-alkylsulfoxyl: C₁-C₆-alkyl-S(═O)—), as used herein refers to a straight-chain or branched saturated hydrocarbon group (as mentioned above) having 1 to 6 carbon atoms bonded through the sulfur atom of the sulfinyl group at any bond in the alkyl group. Examples include C₁-C₆-alkylsulfinyl: S(O)CH₃, S(O)—C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl,

1,2-dimethylpropylsulfinyl, 2,2-dimethylpropylsulfinyl, 1-ethyl propylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethyl butylsulfinyl, 2-ethyl butylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The term “C₁-C₆-alkylamino” refers to a secondary amino group carrying one alkyl group as defined above e.g. methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino, 1-ethyl-2-methylpropylamino.

The term “di(C₁-C₆-alkyl)amino” refers to a tertiary amino group carrying two alkyl radicals as defined above e.g. dimethylamino, diethylamino, di-n-propylamino, diisopropylamino, N-ethyl-N-methylamino, N-(n-propyl)-N-methylamino, N-(isopropyl)N-methylamino, N-(n-butyl)-N-methylamino, N-(n-pentyl)-N-methylamino, N-(2-butyl)N-methylamino, N-(isobutyl)-N-methylamino, N-(n-pentyl)-N-methylamino, N-(n-propyl)-N-ethylamino, N-(isopropyl)-N-ethylamino, N-(n-butyl)-N-ethylamino, N-(n-pentyl)-N-ethylamino, N-(2-butyl)-N-ethylamino, N-(isobutyl)-N-ethylamino, N-(n-pentyl)-N-ethylamino, etc.

The term “C₁-C₆-alkylsulfonyl” (C₁-C₆-alkyl-S(═O)₂—) as used herein refers to a straight-chain or branched saturated alkyl group having 1 to 6 carbon atoms (as mentioned above) which is bonded via the sulfur atom of the sulfonyl group at any bond in the alkyl group. Examples include C₁-C₆-alkylsulfonyl such as SO₂—CH₃, SO₂—C₂H₅, n-propylsulfonyl, SO₂—CH(CH₃)₂, n-butylsulfonyl, 1-methylpropylsulfonyl, 2-methylpropylsulfonyl, SO₂—C(CH₃)₃, n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl and the like.

The term “C₂-C₆-alkenyl” as used herein and in the alkenyl moieties of C₂-C₆-alkenyloxy, C₂-C₆-alkenylamino, C₂-C₆-alkenylthio, C₂-C₆-alkenylsulfonyl, C₂-C₆-alkenylcarbonyl, C₂-C₆-alkenyloxycarbonyl, and C₂-C₆-alkenylcarbonyloxy refers to a straight-chain or branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

In each alkenyl radical the carbon atoms may carry 1, 2 or 3 radicals R^{#}. In other words, each of the hydrogen atoms in these radicals may independently from the others be replaced by one of the aforementioned radicals R^{#}. In case of R^{#} being halogen usually 1, 2, 3 or all of the hydrogen atoms in said alkyl radical are replaced by halogen, especially by fluorine or chlorine. These radicals are also referred to as haloalkenyl. In case of R^{#} being cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, or C₁-C₆-alkylthio usually 1 or 2 of the hydrogen atoms in said alkyl radical may be replaced by the radical R^{#}.

The term “C₂-C₆-alkenyloxy” as used herein refers to a straight-chain or branched alkenyl group having 2 to 6 carbon atoms (as mentioned above) which is attached via an oxygen atom, such as vinyloxy, allyloxy (propen-3-yloxy), methallyloxy, buten-4-yloxy, etc.

The term “C₂-C₆-alkenylthio” as used herein refers to a straight-chain or branched alkenyl group having 2 to 6 carbon atoms (as mentioned above) which is attached via a sulfur atom, for example vinylsulfanyl, allylsulfanyl(propen-3-ylthio), methallylsufanyl, buten-4-ylsulfanyl, etc.

The term “C₂-C₆-alkenylamino” as used herein refers to a straight-chain or branched alkenyl group having 2 to 6 carbon atoms (as mentioned above) which is attached via a nitrogen atom, for example vinylamino, allylamino (propen-3-ylamino), methallylamino, buten-4-ylamino, etc.

The term “C₂-C₆-alkenylsulfonyl” as used herein refers to a straight-chain or branched alkenyl group having 2 to 6 carbon atoms (as mentioned above) which is attached via a sulfonyl (SO₂) group, for example vinylsulfonyl, allylsulfonyl(propen-3-ylsulfonyl), methallylsulfonyl, buten-4-ylsulfonyl, etc.

The term “C₂-C₆-alkenylcarbonyl” as used herein refers to a straight-chain or branched alkenyl group having 2 to 6 carbon atoms (as mentioned above) which is attached via a carbonyl (C═O) group, for example vinylcarbonyl, allylcarbonyl(propen-3-ylcarbonyl), methallylcarbonyl, buten-4-ylcarbonyl, etc.

The term “C₂-C₆-alkenylcarbonyloxy” as used herein refers to a straight-chain or saturated alkenyl group having 2 to 6 carbon atoms (as mentioned above) which is attached via a carbonyloxy (C(O)O) group, for example vinylcarbonyloxy, allylcarbonyloxy (propen-3-ylcarbonyloxy), methallylcarbonyloxy, buten-4-ylcarbonyloxy, etc.

The term “C₂-C₆-alkenyloxycarbonyl” as used herein refers to a straight-chain or saturated alkenyl group having 2 to 6 carbon atoms (as mentioned above) which is attached via an oxycarbonyl (OC(O)) group, for example vinyloxycarbonyl, allyloxycarbonyl (propen-3-yloxycarbonyl), methallyloxycarbonyl, buten-4-yloxycarbonyl, etc.

The term “C₂-C₆-alkynyl” as used herein and in the alkynyl moieties of C₂-C₆-alkynyloxy, C₂-C₆-alkynylamino, C₂-C₆-alkynylthio, C₂-C₆-alkynylsulfonyl, C₂-C₆-alkynylcarbonyl, C₂-C₆-alkynyloxycarbonyl, and C₁-C₆-alkynylcarbonyloxy refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methyl pent-1-yn-1-yl, 3-methyl pent-1-yn-3-yl, 3-methyl pent-1-yn-4-yl, 3-methyl pent-1-yn-5-yl, 4-methylpent-1-yn-1-yl, 4-methylpent-2-yn-4-yl or 4-methylpent-2-yn-5-yl and the like.

In each alkynyl radical the carbon atoms may carry 1, 2 or 3 radicals R^{#}. In other words, each of the hydrogen atoms in these radicals may independently from the others be replaced by one of the aforementioned radicals R^{#}. In case of R^{#} being halogen usually 1, 2, 3 or all of the hydrogen atoms in said alkyl radical are replaced by halogen, especially by fluorine or chlorine. These radicals are also referred to as haloalkyl. In case of R^{#} being cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, or C₁-C₆-alkylthio usually 1 or 2 of the hydrogen atoms in said alkyl radical may be replaced by the radical R^{#}.

The term “C₂-C₆-alkynyloxy” as used herein refers to a straight-chain or branched alkynyl group having 2 to 6 carbon atoms (as mentioned above) which is attached via an oxygen atom, such as propargyloxy (propyn-3-yloxy), butyn-3-yloxy, butyn-4-yloxy, etc.

The term “C₂-C₆-alkynylthio” as used herein refers to a straight-chain or branched alkynyl group having 2 to 6 carbon atoms (as mentioned above) which is attached via a sulfur atom, for example propargylsulfanyl(propyn-3-ylthio), butyn-3-ylsufanyl, butyn-4-ylsulfanyl, etc.

The term “C₂-C₆-alkynylamino” as used herein refers to a straight-chain or branched alkynyl group having 2 to 6 carbon atoms (as mentioned above) which is attached via a nitrogen atom, for example propargylamino (propyn-3-ylamino), butyn-3-amino, butyn-4-ylamino, etc.

The term “C₂-C₆-alkynylsulfonyl” as used herein refers to a straight-chain or branched alkynyl group having 2 to 6 carbon atoms (as mentioned above) which is attached via a sulfonyl (SO₂) group, for example propargylsulfonyl(propyn-3-yltsulfonyl), butyn-3-ylsulfonyl, butyn-4-ylsulfonyl, etc.

The term “C₂-C₆-alkynylcarbonyl” as used herein refers to a straight-chain or branched alkynyl group having 2 to 6 carbon atoms (as mentioned above) which is attached via a carbonyloxy (C═O) group, for example propargylcarbonyl(propyn-3-ylcarbonyl), butyn-3-ylcarbonyl, butyn-4-ylcarbonyl, etc.

The term “C₂-C₆-alkynylcarbonyloxy” as used herein refers to a straight-chain or branched alkynyl group having 2 to 6 carbon atoms (as mentioned above) which is attached via a carbonyloxy (C(O)O) group, for example propargylcarbonyloxy(propyn-3-ylcarbonyloxy), butyn-3-ylcarbonyloxy, butyn-4-ylcarbonyloxy, etc.

The term “C₂-C₆-alkynyloxycarbonyl” as used herein refers to a straight-chain or branched alkynyl group having 2 to 6 carbon atoms (as mentioned above) which is attached via a oxycarbonyl (OC(O)) group, for example propargyloxycarbonyl (propyn-3-yloxycarbonyl), butyn-3-yloxycarbonyl, butyn-4-yloxycarbonyl, etc.

The term “C₃-C₁₂-cycloalkyl” as used herein refers to a mono- or bi- or polycyclic hydrocarbon radical having 3 to 12 carbon atoms, in particular 3 to 6 carbon atoms. Examples of monocyclic radicals comprise cyclopropyl, cyclobutyl, cyclopentyl, cyclohexyl, cycloheptyl, cyclooctyl, cyclononyl or cyclodecyl. Examples of bicyclic radicals comprise bicyclo[2.2.1]heptyl, bicyclo[3.1.1]heptyl, bicyclo[2.2.2]octyl, and bicyclo[3.2.1]octyl. Examples of tricylcic radicals are adamantyl and homoadamantyl.

Each cycloalkyl radical may carry 1, 2, 3, 4 or 5 of the aforementioned radicals R^{#}. In other words, 1, 2, 3, 4 or 5 of the hydrogen atoms in these radicals may independently from the others be replaced by one of the aforementioned radicals R^{#}. Preferred radicals R^{#} on cycloalkyl are selected from halogen, especially fluorine or chlorine, and C₁-C₆-alkyl.

The term “mono- or bicyclic heteroaromatic ring” as used herein refers to a monocyclic heteroaromatic radical which has 5 or 6 ring members, which may comprise a fused 5, 6 or 7 membered ring thus having a total number of ring members from 8 to 10, wherein in each case 1, 2, 3 or 4 of these ring members are heteroatoms selected, independently from each other, from the group consisting of oxygen, nitrogen and sulfur. The heterocyclic radical may be attached to the remainder of the molecule via a carbon ring member or via a nitrogen ring member. The fused ring comprises C₅-C₇-cycloalkyl, C₅-C₇-cycloalkenyl, or 5 to 7 membered heterocyclyl and phenyl.

Examples for monocyclic 5 to 6 membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

Examples for 5 to 6 membered heteroaromatic rings carrying a fused phenyl ring are quinolinyl, isoquinolinyl, indolyl, indolizinyl, isoindolyl, indazolyl, benzofuryl, benzthienyl, benzo[b]thiazolyl, benzoxazolyl, benzthiazolyl, benzoxazolyl, and benzimidazolyl. Examples for 5 to 6 membered heteroaromatic rings carrying a fused cycloalkenyl ring are dihydroindolyl, dihydroindolizinyl, dihydroisoindolyl, dihydrochinolinyl, dihydroisochinolinyl, chromenyl, chromanyl and the like.

The term “5 to 10 membered heterocyclyl” comprises monocyclic heteroaromatic rings as defined above and nonaromatic saturated or partially unsaturated heterocyclic rings having 5, 6, 7, 8, 9 or 10 ring members. Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, dioxanyl, thiopyranyl, dihydrothiopyranyl, tetrahydrothiopyranyl, morpholinyl, thiazinyl and the like.

The term “5 to 7 membered carbocycle” comprises monocyclic aromatic rings and nonaromatic saturated or partially unsaturated carbocyclic rings having 5, 6 or 7 ring members. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl and the like.

The term “linear (C₁-C₄)-alkandiyl” as used herein refers to methylendiyl, ethane-1,2-diyl, propane-1,3-diyl and butane-1,4-diyl.

In a first embodiment of the invention X is oxygen. Those compounds are being referred to as compounds of the formulae I-A-a for the (oxazolin-2-yl)hydrazino compounds and I-A-b for the (3H-oxazolin-2-yliden)hydrazino compounds.

In another embodiment of the invention X is sulfur. Those compounds are being referred to as compounds of the formulae I-B-a for the (thiazolin-2-yl)hydrazino compounds and I-B-b for the (3H-thiazolin-2-yliden)hydrazino compounds.

In yet another embodiment of the invention X is NR⁴, wherein R⁴ has the meaning defined above. Those compounds are being referred to as compounds of the formulae I-C-a for the (imidazolin-2-yl)hydrazino compounds and I-C-b for the (3H-imidazolin-2-yliden)hydrazino compounds.

As regards the pesticidal activity of the compounds of general formulae Ia and Ib, preference is given to those compounds of the formulae Ia, Ib, or I-A-a, I-A-b, I-B-a, 1-B-b, I-C-a or I-C-b respectively, wherein the variables A, Ar, R¹, R^{2a}, R^{2b}, R^{3a}, R^{3b}, R^{3c} and R^{3d} have independently of each other or more preferably in combination the following meanings:
A is preferably a radical CR⁵R⁶, wherein R⁵ is selected from hydrogen or C₁-C₄-alkyl and R⁶ is selected from hydrogen, halogen, C₁-C₄-alkyl, C₁-C₄-haloalkyl, C₁-C₄-alkoxy-C₁-C₄-alkyl, or phenyl, which is unsubstituted or substituted with any combination of 1, 2, 3, 4 or 5 radicals selected from halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy; preference is also given to compounds Ia and Ib, wherein A is a radical C(R^{5a}R^{6a})—C(R^{5b}R^{6b}), wherein R^{5a}, R^{5b}, R^{6a} and R^{6b} have independently one of the meanings given for R⁵ and R⁶; in particular R^{5a}, R^{5b}, R^{6a} and R^{6b} are hydrogen or one of these radicals is different from hydrogen, in particular this radical is C₁-C₄-alkyl while the others are hydrogen; in particular A is CH₂ or CH₂CH₂, especially CH₂.
Ar is preferably phenyl, which is unsubstituted or may carry any combination of 1, 2, 3, 4 or 5 radicals R^{y1} as defined above. In particular the phenyl carries 1, 2, 3, 4 or 5 radicals R^{y1}, which are, independently of each other, preferably selected from the group consisting of halogen, C₁-C₆-alkyl, C₁-C₆-haloalkyl, C₁-C₆-alkylthio, C₁-C₆-haloalkylthio, C₁-C₆-alkoxy, di(C₁-C₆-alkyl)amino and C₁-C₆-haloalkoxy.
R¹ is preferably phenyl, which is unsubstituted or may carry any combination of 1, 2, 3, 4 or 5 radicals R^{y2} as defined above. In particular the radicals R^{y2} are independently of each other selected from the group consisting of halogen, C₁-C₆-alkyl, C₁-C₆-haloalkyl, C₁-C₆-alkylthio, C₁-C₆-haloalkylthio, C₁-C₆-alkoxy, di(C₁-C₆-alkyl)amino and C₁-C₆-haloalkoxy.
R^{2a}, R^{2b} are preferably selected from the group consisting of hydrogen, C₁-C₄-alkyl, formyl, CN, C₁-C₆-alkylcarbonyl, C₁-C₄-haloalkylcarbonyl, C₁-C₆-alkoxycarbonyl, C₁-C₄-alkoxy-C₁-C₄-alkoxycarbonyl or C₁-C₆-alkylthiocarbonyl; in particular R^{2a} or R^{2b} are selected from hydrogen, C₁-C₄-alkyl, C₁-C₄-alkylcarboxyl, C₁-C₆-alkylcarbonyl or CN; especially R^{2a} or R^{2b} is hydrogen.
   Likewise preferred are compounds, wherein R^{2a} or R^{2b} are selected from benzyl or a 5 or 6 membered hetarylmethyl, wherein the hetaryl moiety contains 1, 2, 3 or 4 heteroatoms selected from oxygen, sulfur and nitrogen as ring members and wherein benzoyl and hetaryl are unsubstituted or substituted as mentioned above.
   Likewise preferred are compounds, wherein R^{2a} or R^{2b} are selected from benzoyl or a 5 or 6 membered hetarylcarbonyl, wherein the hetaryl moiety contains 1, 2, 3 or 4 heteroatoms selected from oxygen, sulfur and nitrogen as ring members and wherein benzoyl and hetaryl are unsubstituted or substituted as mentioned above.
R^{3a}, R^{3b}, R^{3c}, R^{3d} are preferably each hydrogen or one of these radicals may also be C₁-C₄-alkyl.

In compounds I-C-a and I-C-b the radical R⁴ is preferably selected from the group consisting of hydrogen, formyl, C₁-C₆-alkyl, C₁-C₆-alkylcarbonyl, C₁-C₆-alkoxycarbonyl, phenyl, benzyl, phenoxycarbonyl and benzoyl each of the last four mentioned radicals may be unsubstituted or may carry any combination of 1, 2, 3, 4 or 5 radicals R^{b4} in particular 1, 2 or 3 halogen atoms or 1 radical R^{b4} different from halogen; especially R⁴ is selected from hydrogen, C₁-C₆-alkyl, C₁-C₆-alkylcarbonyl, phenyl or benzyl.

Preference is also given to those compounds of formulae I-A-a, I-A-b, I-B-a, I-B-b, I-C-a or I-C-b, wherein the variables A, Ar, R^{2a}, R^{2b}, R^{3a}, R^{3b}, R^{3c}, R^{3d} and R⁴ have independently of each other or more preferably in combination the meanings given above, and R¹ is selected from the group consisting of
CN,
C₃-C₁₀-cycloalkyl, which is unsubstituted or may carry 1, 2 or 3 radicals R^{b1},
C₂-C₆-alkenyl, which is unsubstituted or may carry 1, 2 or 3 radicals R^{c1},
C₂-C₆-alkynyl, which is unsubstituted or may carry 1, 2 or 3 radicals R^{c1},
a radical O—R^{z1},
a radical S(O)ₘ—R^{z2} with m being 0, 1 or 2, or
a radical C(O)—R^{z3}.

More preference is given to those compounds wherein R¹ is selected from the group consisting of
CN,
C₃-C₁₀-cycloalkyl, which may be unsubstituted or may carry 1, 2, 3, 4 or 5 radicals R^{b1},
C₂-C₆-alkenyl,
C₂-C₆-alkynyl,
a radical O-R^{z1},
a radical S(O)ₘ—R^{z2} with m being 0, 1 or 2, and
a radical C(O)—R^{z3}.

Preference is also given to those compounds of formulae I-A-a, I-A-b, I-B-a, I-B-b, I-C-a or I-C-b, wherein the variables A, Ar, R^{2a}, R^{2b}, R^{3a}, R^{3b}, R^{3c}, R^{3d} and R⁴ have independently of each other or more preferably in combination the meanings given above, and R¹ is C₁-C₆-alkyl, which may carry 1, 2 or 3 radicals R^{a1}, in particular 1, 2 or 3 halogen atoms or 1 radical R^{a1} different from halogen.

In a further embodiment A is C(O) and R¹ is a radical O-R^{z1} or NR^{z4}R^{z5}. Preference is given to those compounds, wherein the substituent radicals have the following meaning:
R^{z1} is selected from the group consisting of C₁-C₄-alkyl, C₁-C₄-haloalkyl, C₁-C₄-alkoxy-C₁-C₄-alkyl, phenyl and benzyl, each of the last two mentioned radicals may be unsubstituted or may carry any combination of 1, 2 or 3 radicals R^{b}, especially 1 or 2 radicals R^{b}.
R^{z4} and R^{z5} are independently from each other selected from the group consisting of hydrogen, C₁-C₆-alkyl, C₂-C₆-alkenyl, wherein the carbon atoms in these groups may carry any combination of 1, 2 or 3 radicals R^{az}, in particular 1, 2 or 3 halogen atoms or 1 radical R^{az} different from halogen, or phenyl, phenyl-C₁-C₄-alkyl, benzoyl, wherein the last three mentioned groups may carry any combination of 1, 2, 3, 4 or 5 radicals R^{bz}.

In a further embodiment R¹ together with R^{2a} forms a linear C₂-C₄-alkandiyl, which may carry 1, 2, 3 or 4 radicals independently selected from halogen, C₁-C₄-alkyl and C₁-C₄-haloalkyl, wherein the radicals which are bound to adjacent carbon atoms of alkylene may together with the carbon atoms to which they are bound, form a fused phenyl ring, which may carry 1, 2, 3 or 4 radicals independently selected from halogen, C₁-C₄-alkyl and C₁-C₄-haloalkyl. In particular, the fused phenyl ring is unsubstituted or carries 1, 2 or 3 halogen atoms and/or 1 radical different from halogen. In this embodiment, one or two, in particular one CH₂ moieties of the linear C₂-C₄-alkandiyl may be replaced by O or NR^{q} as defined above. The one or two CH₂ moieties of the linear C₂-C₄-alkandiyl, which are replaced, are not adjacent and preferably also not adjacent to the nitrogen atom that carries R^{2a}.

Apart from the foregoing the substituent radicals R^{x}, R^{z1}, R^{z2}, R^{z3}, R^{a1}, R^{a2}, R^{a3}, R^{a4}, R^{ax}, R^{ay}, R^{az}, R^{b1}, R^{b2}, R^{b3}, R^{b4}, R^{bx}, R^{by}, R^{bz}, R^{a}, R^{b}, R^{c} and R^{d}, if present have preferably the following meanings:

R^{x}, if present, is preferably selected from the group consisting of halogen, C₁-C₄-alkyl, C₁-C₄-haloalkyl, C₁-C₄-alkoxy-C₁-C₄-alkyl, and phenyl, which is unsubstituted or substituted with any combination of 1, 2, 3, 4 or 5 radicals selected from halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy. Preferably R^{x} is not present, i.e. A is unsubstituted alkylene, or A carries only one radical R^{x} is different from hydrogen. More preferably R^{x}, if present, is C₁-C₄-alkyl in particular methyl.

R^{z1}, if present, is preferably selected from the group consisting of C₁-C₄-alkyl, C₁-C₄-haloalkyl, C₁-C₄-alkoxy-C₁-C₄-alkyl, phenyl and benzyl, each of the last two mentioned radicals may be unsubstituted or may carry any combination of 1, 2 or 3 radicals R^{bz}, especially 1 or 2 radicals R^{bz}.

R^{z2}, if present, is/are independently selected from the group consisting of C₁-C₆-alkyl, C₁-C₆-haloalkyl and phenyl, which may be unsubstituted or may carry any combination of 1, 2, 3 radicals selected from the group consisting of halogen, C₁-C₆-alkyl, C₁-C₆-haloalkyl, C₁-C₆-alkoxy and C₁-C₆-haloalkoxy, in particular 1, 2 or 3 halogen atoms or 1 radical different from halogen.

R^{z3}, if present, is/are independently selected from the group consisting of C₁-C₆-alkyl, C₁-C₆-haloalkyl, C₁-C₆-alkoxy, C₁-C₆-haloalkoxy, a radical NR^{c}R^{d}, phenyl, benzyl and phenoxy, each of the last three mentioned radicals may be unsubstituted or may carry any combination of 1, 2 or 3 radicals R^{bz}, preferably selected independently from halogen, C₁-C₆-alkyl, C₁-C₆-haloalkyl, C₁-C₆-alkoxy and C₁-C₆-haloalkoxy, in particular 1, 2 or 3 halogen atoms or 1 radical R^{bz} different from halogen.

If present the radical(s) R^{a1} is/are independently selected from the group consisting of halogen, cyano, C₃-C₆-cycloalkyl, C₁-C₆-alkoxy and C₁-C₆-haloalkoxy.

R^{a2}, R^{a3}, R^{a4}, R^{ax} and R^{ay}, if present, are independently selected from the group consisting of halogen, cyano, C₁-C₆-haloalkoxy and C₁-C₆-alkoxy.

R^{b1}, if present, is/are independently selected from the group consisting of halogen, cyano, nitro, C₁-C₆-alkyl, C₁-C₆-haloalkyl, C₁-C₆-haloalkoxy and C₁-C₆-alkoxy.

R^{b2}, R^{b3}, R^{b4}, R^{bx} and R^{by}, if present, are independently selected from the group consisting of halogen, cyano, nitro, C₁-C₆-alkyl, C₁-C₆-haloalkyl, C₁-C₆-haloalkoxy and C₁-C₆-alkoxy.

R^{c1}, if present, is/are independently selected from the group consisting of hydrogen, halogen, C₁-C₆-alkyl and C₁-C₆-haloalkyl.

If present, R^{az} is/are preferably selected from the group consisting of halogen, cyano, nitro, C₃-C₆-cycloalkyl, C₁-C₆-alkoxy, C₁-C₆-haloalkoxy, C₁-C₆-alkylcarbonyl, C₁-C₆-alkoxycarbonyl and C₁-C₆-alkylsulfonyl.

If present, R^{bz} is/are preferably selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, di(C₁-C₄-alkyl)amino, C₁-C₄-alkylsulfonyl, C₁-C₄-alkylcarbonyl and C₁-C₄-alkoxycarbonyl.

If present, R^{a} and R^{b} are, independently of each other, preferably selected from the group consisting of hydrogen, C₁-C₆-alkyl and C₂-C₆-alkenyl.

If present, R^{c} and R^{d} are, independently of each other, preferably selected from the group consisting of C₁-C₆-alkyl or C₂-C₆-alkenyl, wherein the carbon atoms in these groups may carry any combination of 1, 2 or 3 radicals Ray, preferably selected from halogen, cyano, nitro, C₁-C₆-alkoxy and C₁-C₆-haloalkoxy. In particular R^{c} and R^{d} are unsubstituted or carry 1, 2 or 3 halogen atoms or 1 radical Ray different from halogen.

Preference is also given to those compounds of formulae I-A-a, I-A-b, I-B-a, I-B-b, I-C-a or I-C-b, wherein the variables A, R^{2a}, R^{2b}, R^{3a}, R^{3b}, R^{3c}, R^{3d} and R⁴ have independently of each other or more preferably in combination the meanings given above, and the radicals Ar and/or R¹ are selected independently of each other from:

### a) Phenyl:

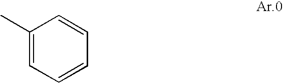

b) Monosubstituted phenyl radicals:

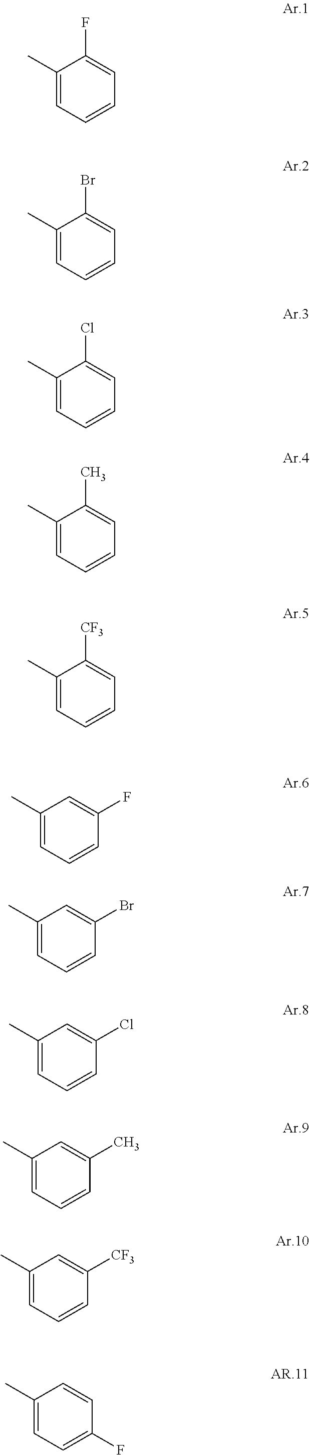

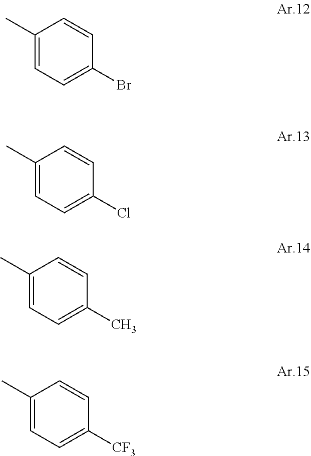

c) disubstituted phenyl radicals:

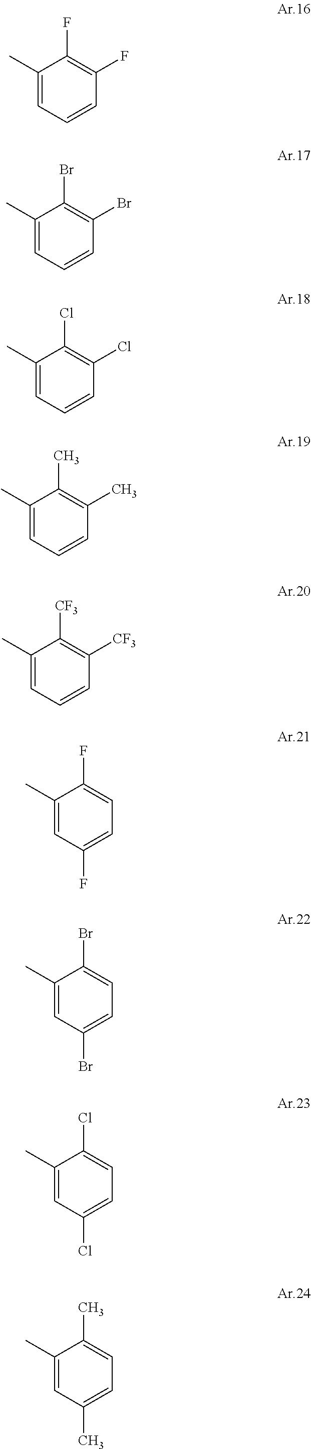

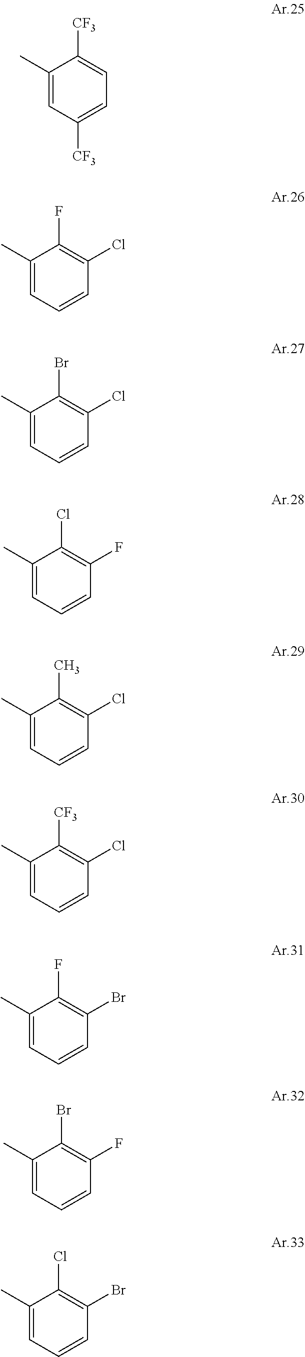

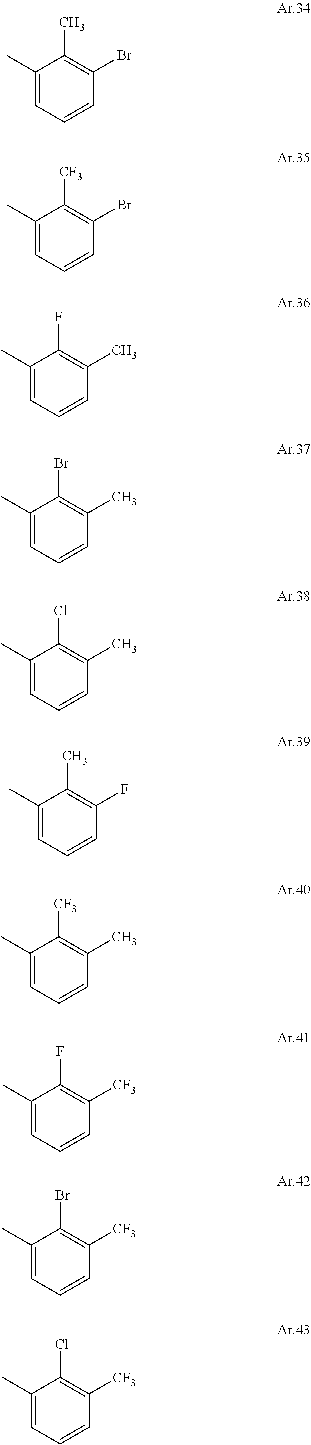

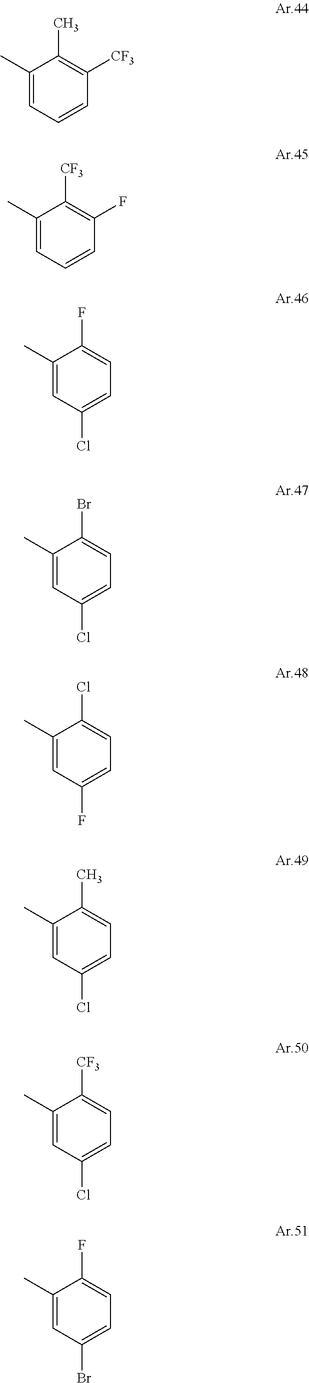

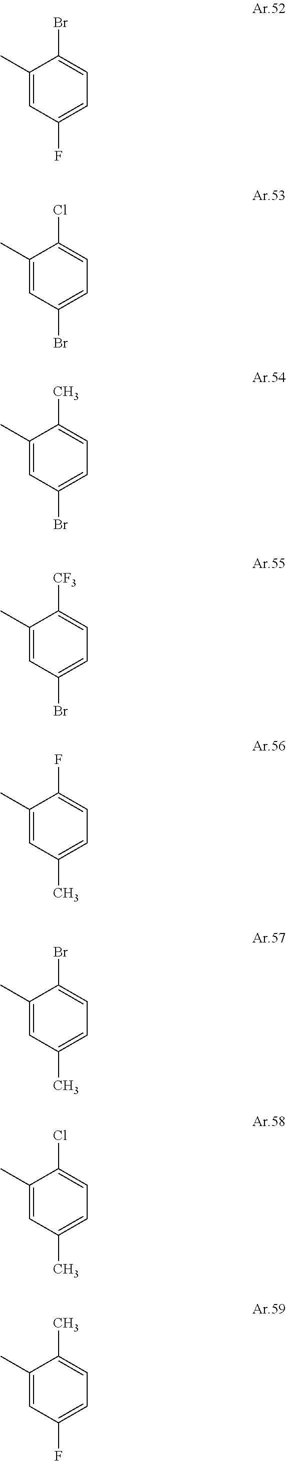

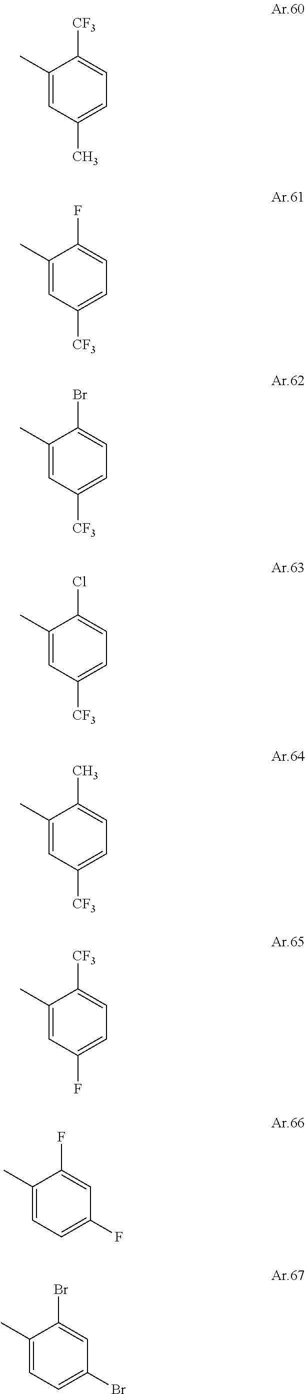

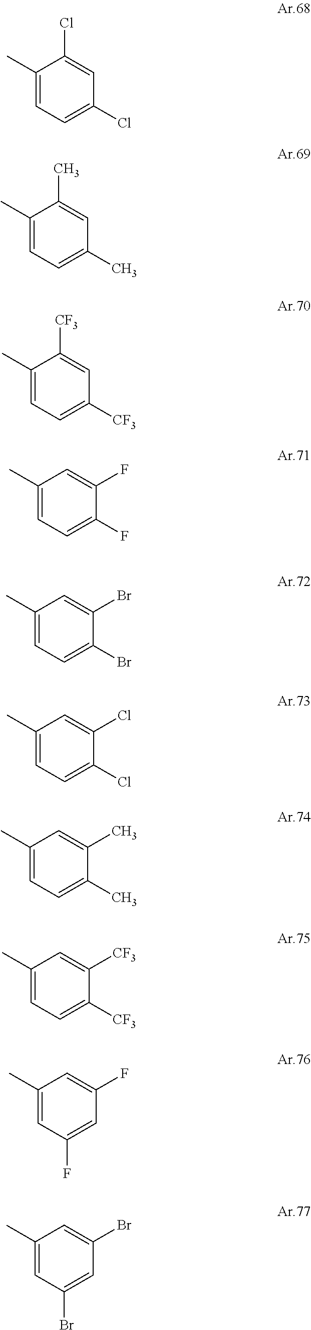

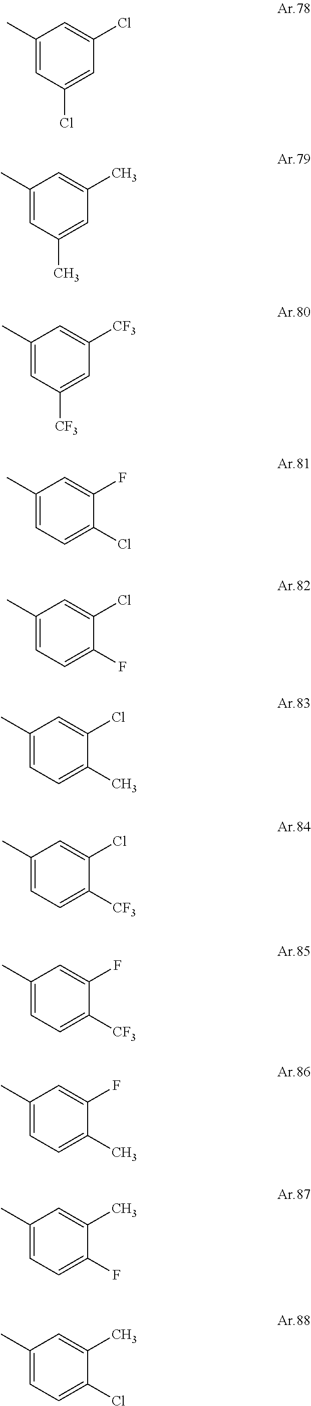

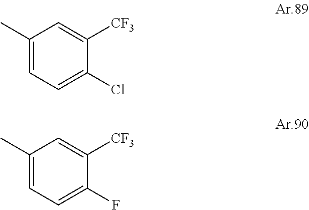

d) and trisubstituted phenyl radicals:

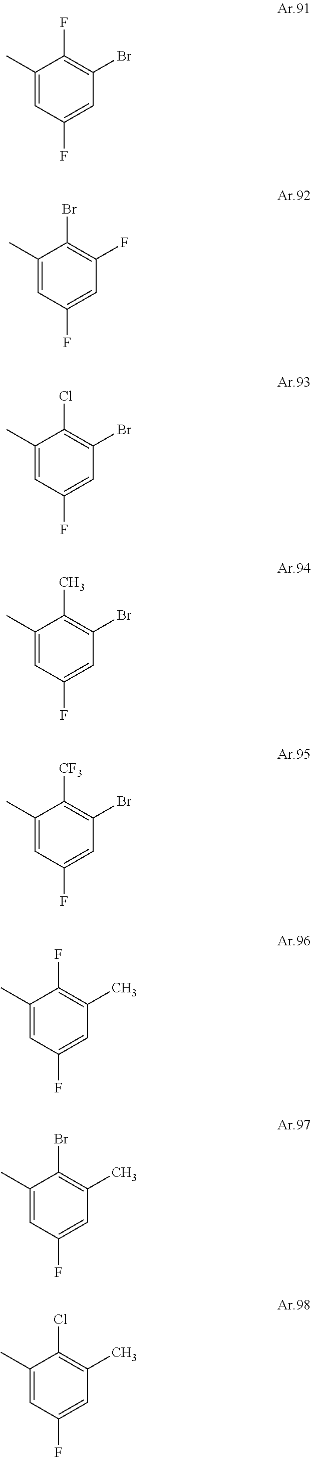

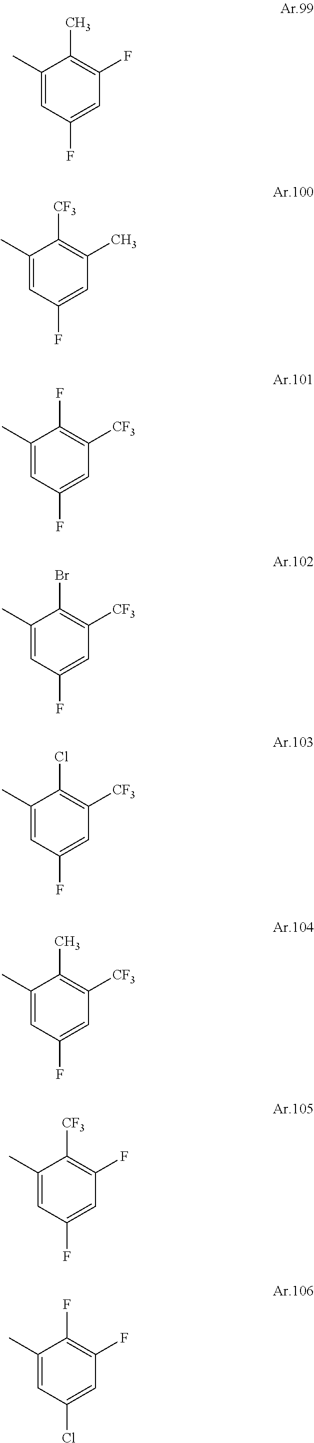

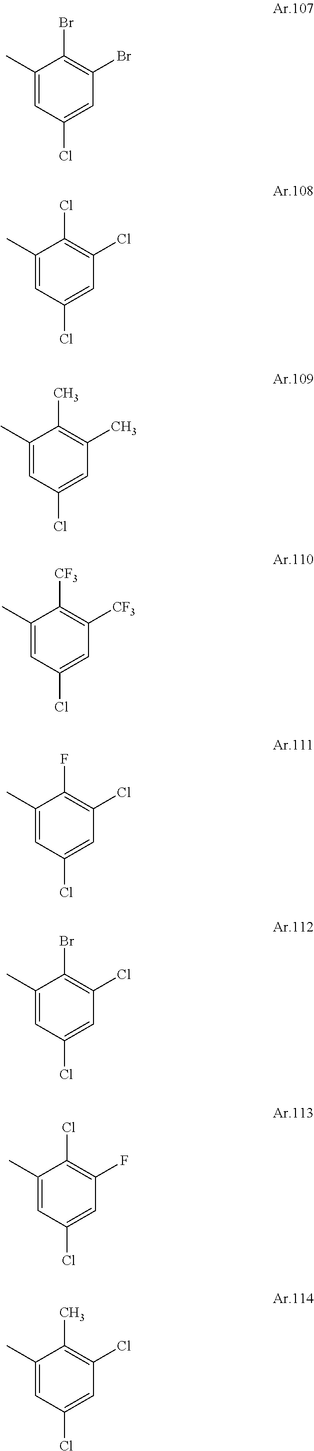

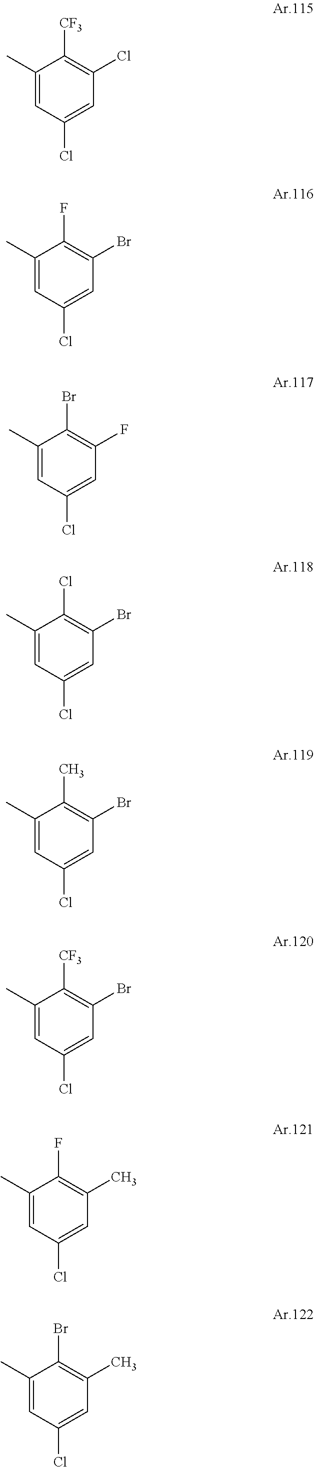

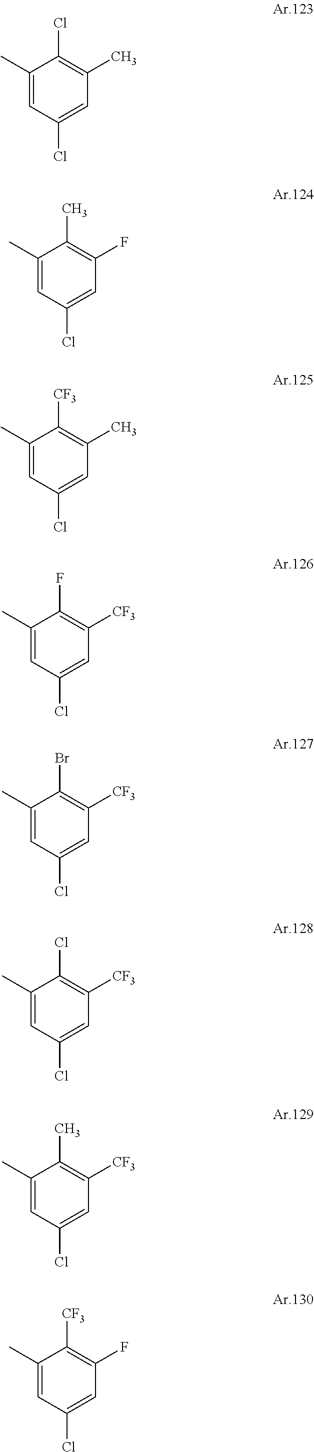

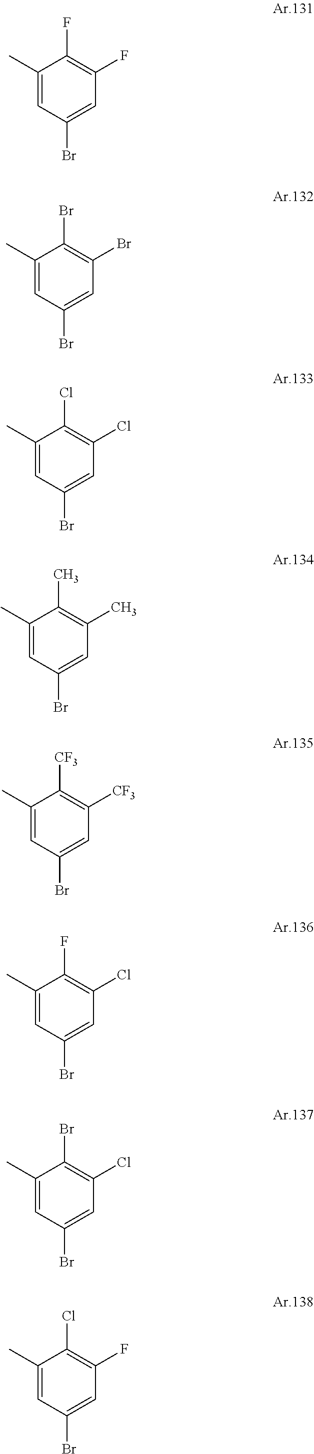

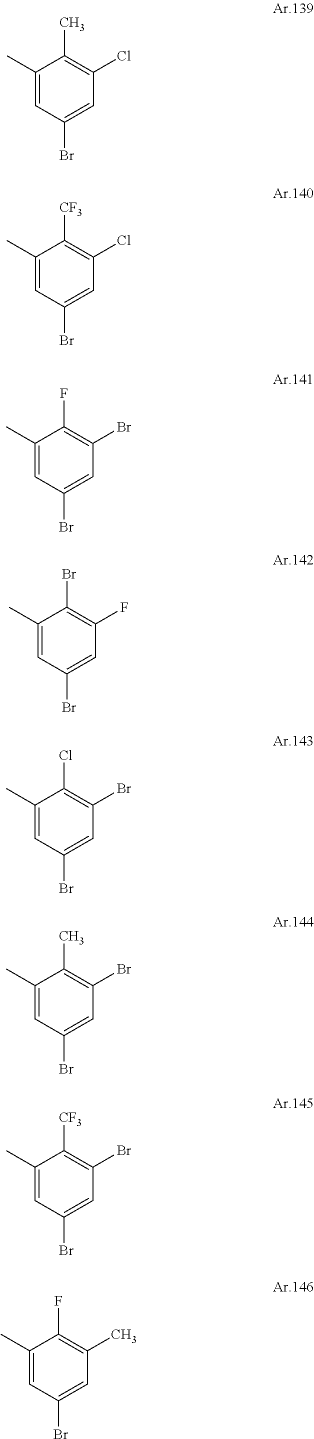

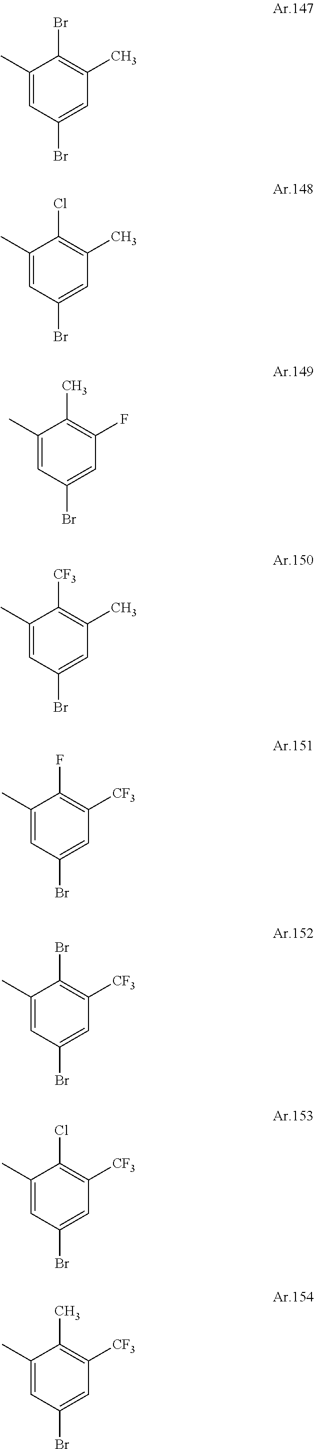

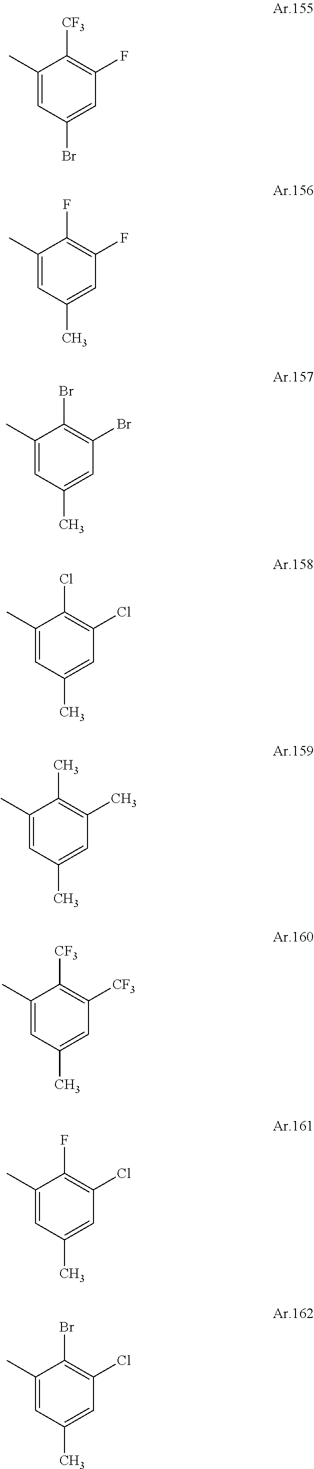

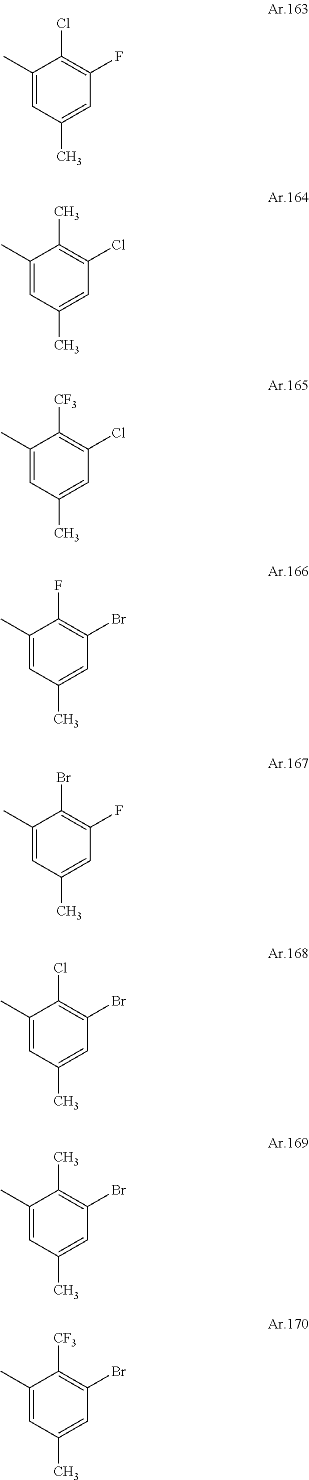

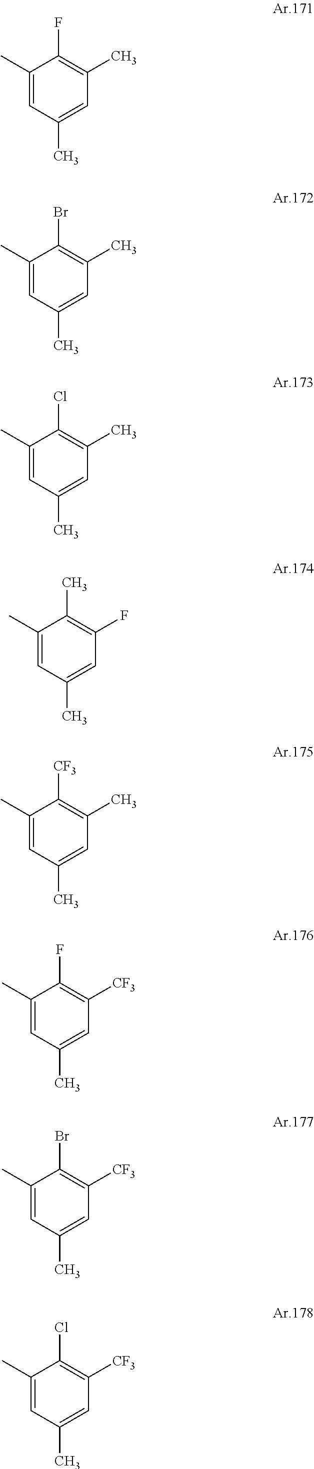

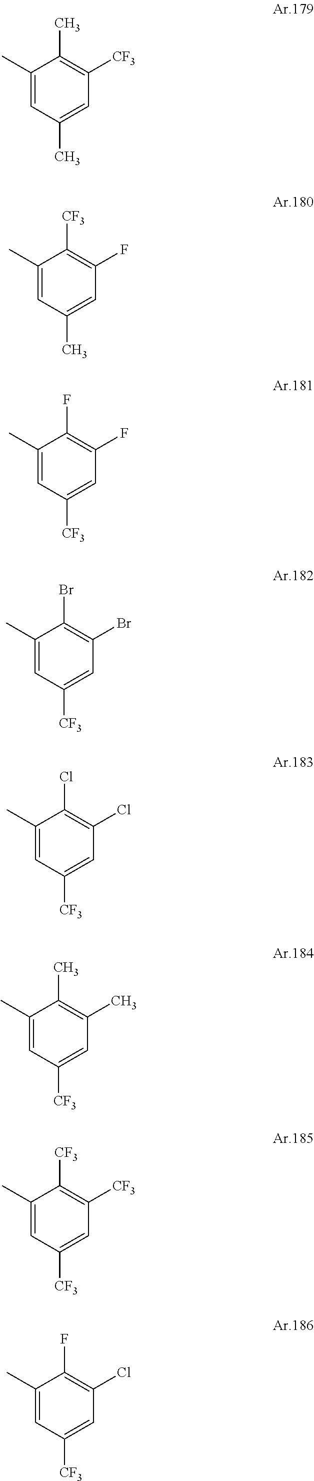

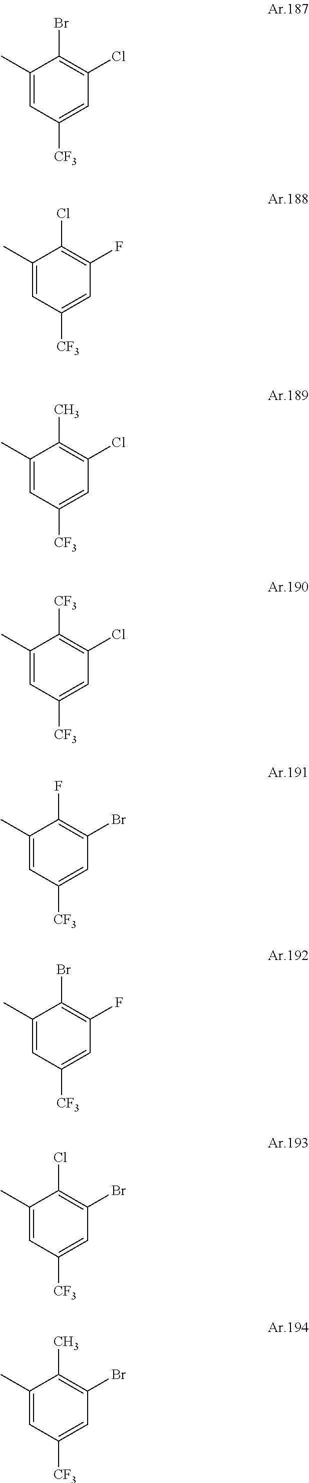

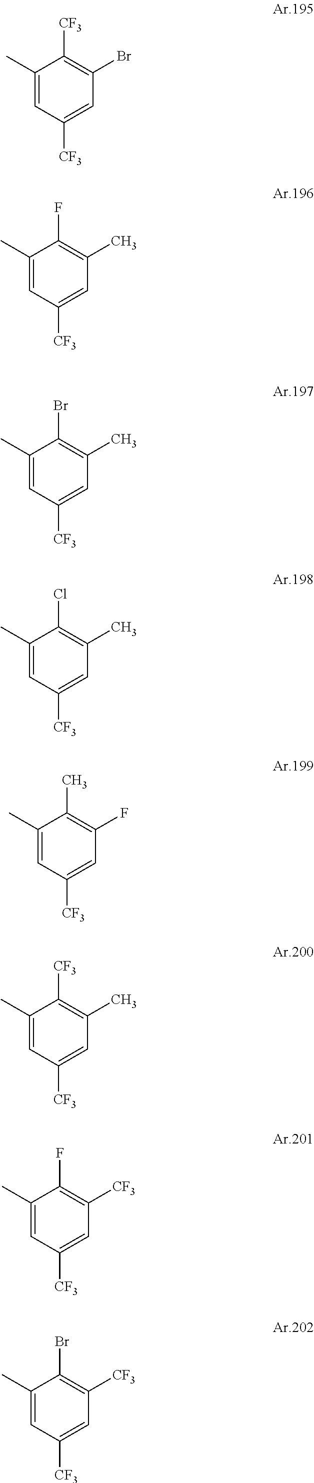

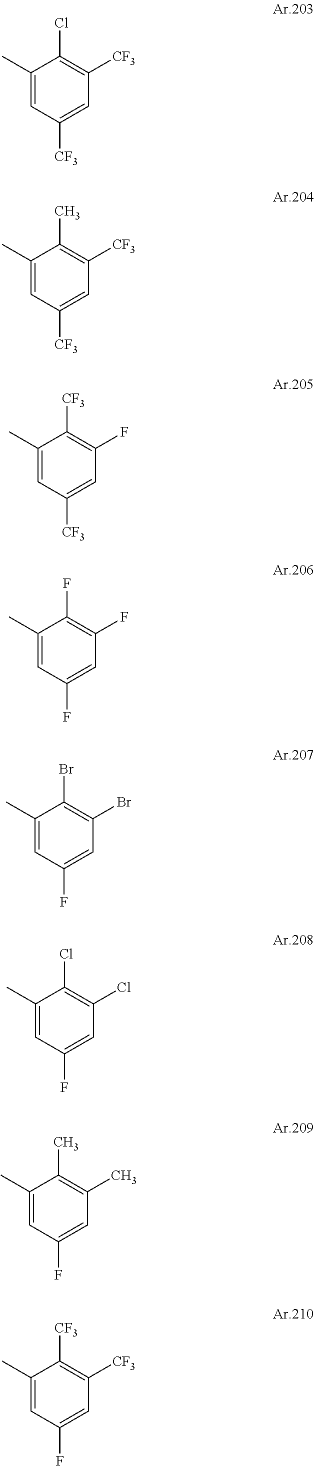

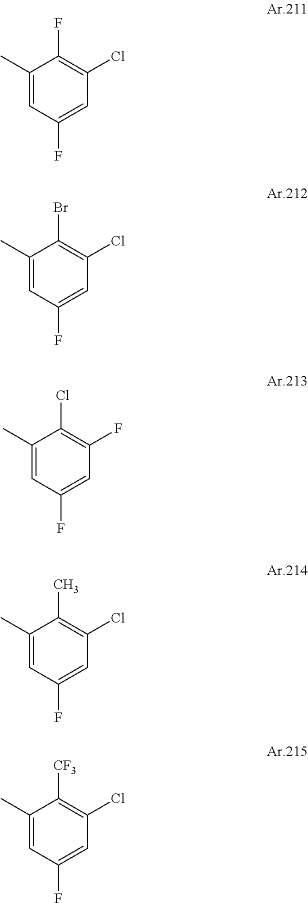

e) 5 or 6 membered heteroaromatic rings

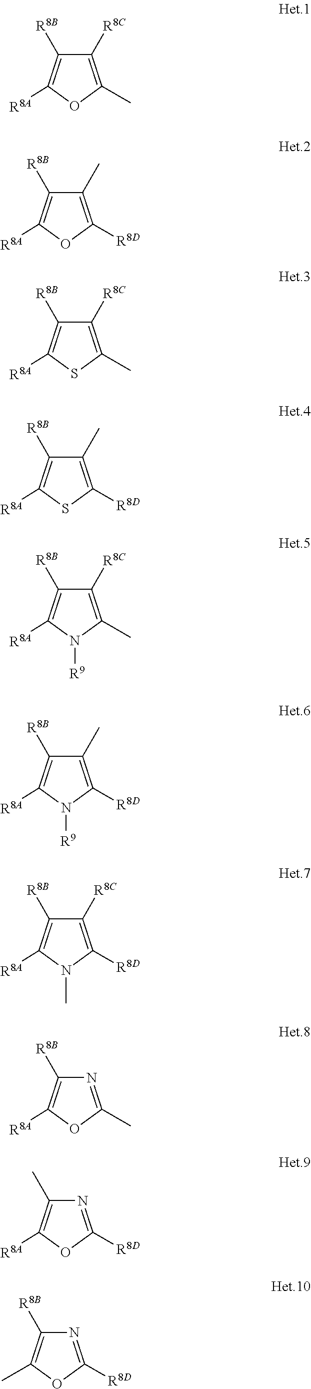

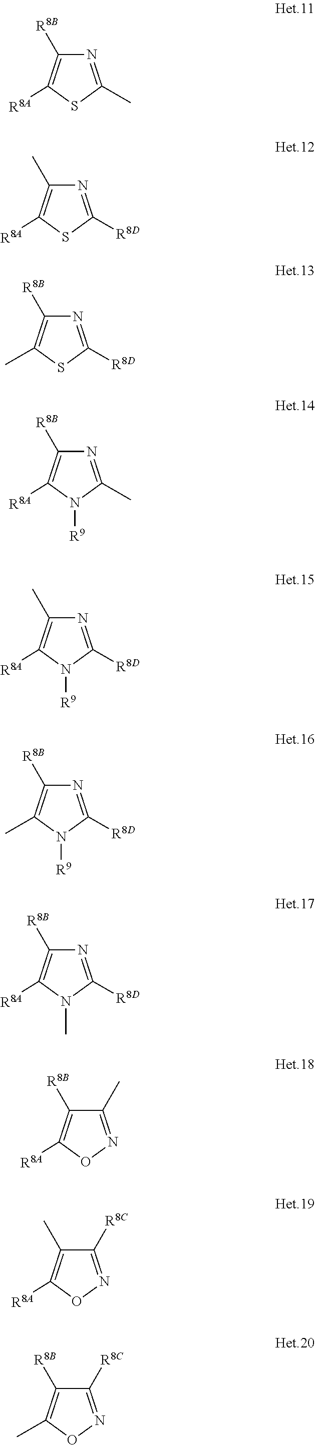

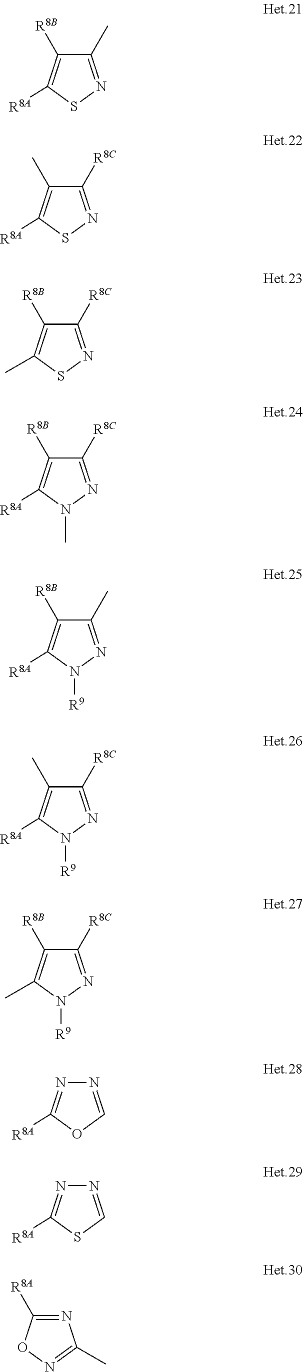

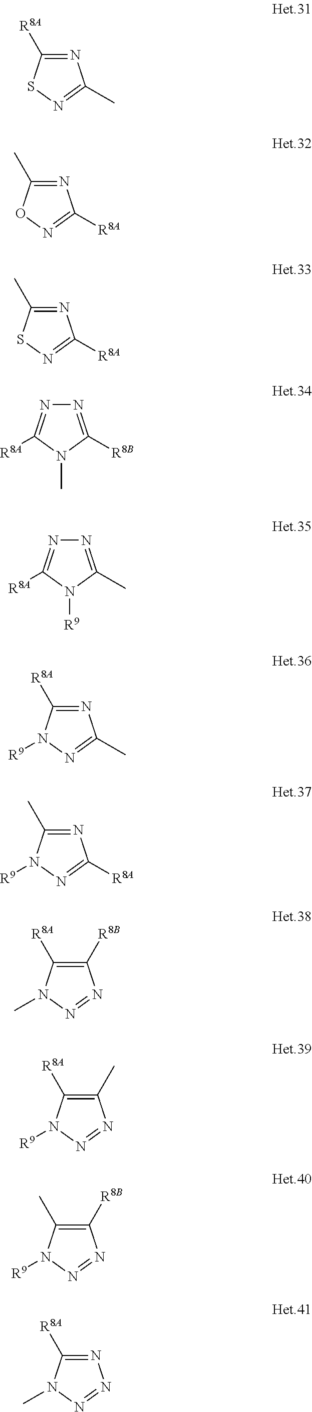

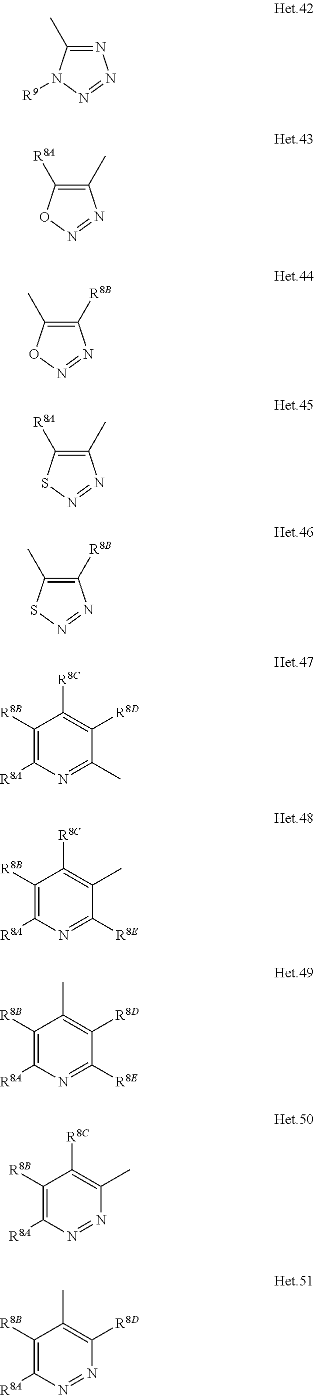

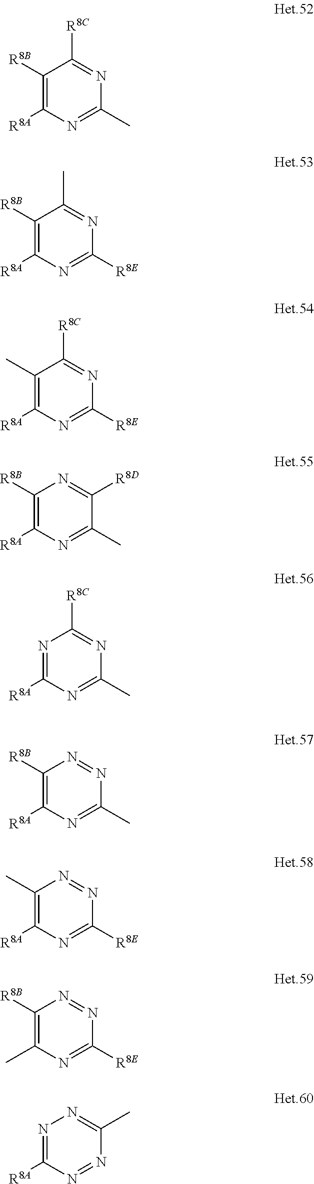

wherein the radicals R^{8A}, R^{8B}, R^{8C}, R^{8D}, R^{8E} and R⁹ have the meaning given for R^{y1}, R^{y2} and R^{y3}, and wherein the free bond denotes the position of attachment in formulae I-A-a, I-A-b, I-B-a, I-B-b, I-C-a or I-C-b.

Especially preferred are compounds, wherein the radicals Ar and/or R¹ are selected from Het.1, Het.2, Het.3, Het.4, Het.5, Het.6, Het.8, Het.9, Het.10, Het.11, Het.12, Het.13, Het.14, Het.15, Het.17, Het.18, Het.19, Het.20, Het.21, Het.22, Het.23, Het.25, Het.26, Het.27, Het.28, Het.29, Het.30, Het.31, Het.32, Het.33, Het.47, Het.48, Het.49, Het.52, Het.53 and Het.54.

Preferred are also compounds, wherein the radicals Ar and/or R¹ are selected from Het.1, Het.3, Het. 25, Het.26, Het.27, Het.47, Het.48, Het.49, Het.52, Het.53 and Het.54.

Preferred are also compounds of formulae I-A-a, I-A-b, I-B-a, I-B-b, I-C-a or I-C-b,

wherein Ar and/or R¹ are selected from the following table A of monosubstituted heteroarylradicals (Het-R.1-Het-R.512):

TABLE A

| Radical | Het | R^{8A} | R^{8B} | R^{8C} | R^{8D} | R^{8E} | R⁹ |
|---|---|---|---|---|---|---|---|
| Het-R.1 | Het.1 | H | H | H | / | / | / |
| Het-R.2 | Het.1 | CH₃ | H | H | / | / | / |
| Het-R.3 | Het.1 | H | CH₃ | H | / | / | / |
| Het-R.4 | Het.1 | H | H | CH₃ | / | / | / |
| Het-R.5 | Het.1 | CF₃ | H | H | / | / | / |
| Het-R.6 | Het.1 | H | CF₃ | H | / | / | / |
| Het-R.7 | Het.1 | H | H | CF₃ | / | / | / |
| Het-R.8 | Het.1 | Cl | H | H | / | / | / |
| Het-R.9 | Het.1 | H | Cl | H | / | / | / |
| Het-R.10 | Het.1 | H | H | Cl | / | / | / |
| Het-R.11 | Het.2 | H | H | / | H | / | / |
| Het-R.12 | Het.2 | CH₃ | H | / | H | / | / |
| Het-R.13 | Het.2 | H | CH₃ | / | H | / | / |
| Het-R.14 | Het.2 | H | H | / | CH₃ | / | / |
| Het-R.15 | Het.2 | CF₃ | H | / | H | / | / |
| Het-R.16 | Het.2 | H | CF₃ | / | H | / | / |
| Het-R.17 | Het.2 | H | H | / | CF₃ | / | / |
| Het-R.18 | Het.2 | Cl | H | / | H | / | / |
| Het-R.19 | Het.2 | H | Cl | / | H | / | / |
| Het-R.20 | Het.2 | H | H | / | Cl | / | / |
| Het-R.21 | Het.3 | H | H | H | / | / | / |
| Het-R.22 | Het.3 | CH₃ | H | H | / | / | / |
| Het-R.23 | Het.3 | H | CH₃ | H | / | / | / |
| Het-R.24 | Het.3 | H | H | CH₃ | / | / | / |
| Het-R.25 | Het.3 | CF₃ | H | H | / | / | / |
| Het-R.26 | Het.3 | H | CF₃ | H | / | / | / |
| Het-R.27 | Het.3 | H | H | CF₃ | / | / | / |
| Het-R.28 | Het.3 | Cl | H | H | / | / | / |
| Het-R.29 | Het.3 | H | Cl | H | / | / | / |
| Het-R.30 | Het.3 | H | H | Cl | / | / | / |
| Het-R.31 | Het.4 | H | H | / | H | / | / |
| Het-R.32 | Het.4 | CH₃ | H | / | H | / | / |
| Het-R.33 | Het.4 | H | CH₃ | / | H | / | / |
| Het-R.34 | Het.4 | H | H | / | CH₃ | / | / |
| Het-R.35 | Het.4 | CF₃ | H | / | H | / | / |
| Het-R.36 | Het.4 | H | CF₃ | / | H | / | / |
| Het-R.37 | Het.4 | H | H | / | CF₃ | / | / |
| Het-R.38 | Het.4 | Cl | H | / | H | / | / |
| Het-R.39 | Het.4 | H | Cl | / | H | / | / |
| Het-R.40 | Het.4 | H | H | / | Cl | / | / |
| Het-R.41 | Het.5 | H | H | H | / | / | H |
| Het-R.42 | Het.5 | CH₃ | H | H | / | / | H |
| Het-R.43 | Het.5 | H | CH₃ | H | / | / | H |
| Het-R.44 | Het.5 | H | H | CH₃ | / | / | H |
| Het-R.45 | Het.5 | CF₃ | H | H | / | / | H |
| Het-R.46 | Het.5 | H | CF₃ | H | / | / | H |
| Het-R.47 | Het.5 | H | H | CF₃ | / | / | H |
| Het-R.48 | Het.5 | Cl | H | H | / | / | H |
| Het-R.49 | Het.5 | H | Cl | H | / | / | H |
| Het-R.50 | Het.5 | H | H | Cl | / | / | H |
| Het-R.51 | Het.5 | H | H | H | / | / | CH₃ |
| Het-R.52 | Het.5 | CH₃ | H | H | / | / | CH₃ |
| Het-R.53 | Het.5 | H | CH₃ | H | / | / | CH₃ |
| Het-R.54 | Het.5 | H | H | CH₃ | / | / | CH₃ |
| Het-R.55 | Het.5 | CF₃ | H | H | / | / | CH₃ |
| Het-R.56 | Het.5 | H | CF₃ | H | / | / | CH₃ |
| Het-R.57 | Het.5 | H | H | CF₃ | / | / | CH₃ |
| Het-R.58 | Het.5 | Cl | H | H | / | / | CH₃ |
| Het-R.59 | Het.5 | H | Cl | H | / | / | CH₃ |
| Het-R.60 | Het.5 | H | H | Cl | / | / | CH₃ |
| Het-R.61 | Het.6 | H | H | / | H | / | H |
| Het-R.62 | Het.6 | CH₃ | H | / | H | / | H |
| Het-R.63 | Het.6 | H | CH₃ | / | H | / | H |
| Het-R.64 | Het.6 | H | H | / | CH₃ | / | H |
| Het-R.65 | Het.6 | CF₃ | H | / | H | / | H |
| Het-R.66 | Het.6 | H | CF₃ | / | H | / | H |
| Het-R.67 | Het.6 | H | H | / | CF₃ | / | H |
| Het-R.68 | Het.6 | Cl | H | / | H | / | H |
| Het-R.69 | Het.6 | H | Cl | / | H | / | H |
| Het-R.70 | Het.6 | H | H | / | Cl | / | H |
| Het-R.71 | Het.6 | H | H | / | H | / | CH₃ |
| Het-R.72 | Het.6 | CH₃ | H | / | H | / | CH₃ |
| Het-R.73 | Het.6 | H | CH₃ | / | H | / | CH₃ |
| Het-R.74 | Het.6 | H | H | / | CH₃ | / | CH₃ |
| Het-R.75 | Het.6 | CF₃ | H | / | H | / | CH₃ |
| Het-R.76 | Het.6 | H | CF₃ | / | H | / | CH₃ |
| Het-R.77 | Het.6 | H | H | / | CF₃ | / | CH₃ |
| Het-R.78 | Het.6 | Cl | H | / | H | / | CH₃ |
| Het-R.79 | Het.6 | H | Cl | / | H | / | CH₃ |
| Het-R.80 | Het.6 | H | H | / | Cl | / | CH₃ |
| Het-R.81 | Het.7 | H | H | H | H | / | / |
| Het-R.82 | Het.7 | CH₃ | H | H | H | / | / |
| Het-R.83 | Het.7 | H | CH₃ | H | H | / | / |
| Het-R.84 | Het.7 | H | H | CH₃ | H | / | / |
| Het-R.85 | Het.7 | H | H | H | CH₃ | / | / |
| Het-R.86 | Het.7 | CF₃ | H | H | H | / | / |
| Het-R.87 | Het.7 | H | CF₃ | H | H | / | / |
| Het-R.88 | Het.7 | H | H | CF₃ | H | / | / |
| Het-R.89 | Het.7 | H | H | H | CF₃ | / | / |
| Het-R.90 | Het.7 | Cl | H | H | H | / | / |
| Het-R.91 | Het.7 | H | Cl | H | H | / | / |
| Het-R.92 | Het.7 | H | H | Cl | H | / | / |
| Het-R.93 | Het.7 | H | H | H | Cl | / | / |
| Het-R.94 | Het.8 | H | H | / | / | / | / |
| Het-R.95 | Het.8 | H | CH₃ | / | / | / | / |
| Het-R.96 | Het.8 | CH₃ | H | / | / | / | / |
| Het-R.97 | Het.8 | H | CF₃ | / | / | / | / |
| Het-R.98 | Het.8 | CF₃ | H | / | / | / | / |
| Het-R.99 | Het.8 | H | Cl | / | / | / | / |
| Het-R.100 | Het.8 | Cl | H | / | / | / | / |
| Het-R.101 | Het.9 | H | / | / | H | / | / |
| Het-R.102 | Het.9 | CH₃ | / | / | H | / | / |
| Het-R.103 | Het.9 | H | / | / | CH₃ | / | / |
| Het-R.104 | Het.9 | CF₃ | / | / | H | / | / |
| Het-R.105 | Het.9 | H | / | / | CF₃ | / | / |
| Het-R.106 | Het.9 | Cl | / | / | H | / | / |
| Het-R.107 | Het.9 | H | / | / | Cl | / | / |
| Het-R.108 | Het.10 | / | H | / | H | / | / |
| Het-R.109 | Het.10 | / | H | / | CH₃ | / | / |
| Het-R.110 | Het.10 | / | CH₃ | / | H | / | / |
| Het-R.111 | Het.10 | / | H | / | CF₃ | / | / |
| Het-R.112 | Het.10 | / | CF₃ | / | H | / | / |
| Het-R.113 | Het.10 | / | H | / | Cl | / | / |
| Het-R.114 | Het.10 | / | Cl | / | H | / | / |
| Het-R.115 | Het.11 | H | H | / | / | / | / |
| Het-R.116 | Het.11 | H | CH₃ | / | / | / | / |
| Het-R.117 | Het.11 | CH₃ | H | / | / | / | / |
| Het-R.118 | Het.11 | H | CF₃ | / | / | / | / |
| Het-R.119 | Het.11 | CF₃ | H | / | / | / | / |
| Het-R.120 | Het.11 | H | Cl | / | / | / | / |
| Het-R.121 | Het.11 | Cl | H | / | / | / | / |
| Het-R.122 | Het.12 | H | / | / | H | / | / |
| Het-R.123 | Het.12 | CH₃ | / | / | H | / | / |
| Het-R.124 | Het.12 | H | / | / | CH₃ | / | / |
| Het-R.125 | Het.12 | CF₃ | / | / | H | / | / |
| Het-R.126 | Het.12 | H | / | / | CF₃ | / | / |
| Het-R.127 | Het.12 | Cl | / | / | H | / | / |
| Het-R.128 | Het.12 | H | / | / | Cl | / | / |
| Het-R.129 | Het.13 | / | H | / | H | / | / |
| Het-R.130 | Het.13 | / | H | / | CH₃ | / | / |
| Het-R.131 | Het.13 | / | CH₃ | / | H | / | / |
| Het-R.132 | Het.13 | / | H | / | CF₃ | / | / |
| Het-R.133 | Het.13 | / | CF₃ | / | H | / | / |
| Het-R.134 | Het.13 | / | H | / | Cl | / | / |
| Het-R.135 | Het.13 | / | Cl | / | H | / | / |
| Het-R.136 | Het.14 | H | H | / | / | / | H |
| Het-R.137 | Het.14 | H | CH₃ | / | / | / | H |
| Het-R.138 | Het.14 | CH₃ | H | / | / | / | H |
| Het-R.139 | Het.14 | H | CF₃ | / | / | / | H |
| Het-R.140 | Het.14 | CF₃ | H | / | / | / | H |
| Het-R.141 | Het.14 | H | Cl | / | / | / | H |
| Het-R.142 | Het.14 | Cl | H | / | / | / | H |
| Het-R.143 | Het.14 | H | H | / | / | / | CH₃ |
| Het-R.144 | Het.14 | H | CH₃ | / | / | / | CH₃ |
| Het-R.145 | Het.14 | CH₃ | H | / | / | / | CH₃ |
| Het-R.146 | Het.14 | H | CF₃ | / | / | / | CH₃ |
| Het-R.147 | Het.14 | CF₃ | H | / | / | / | CH₃ |
| Het-R.148 | Het.14 | H | Cl | / | / | / | CH₃ |
| Het-R.149 | Het.14 | Cl | H | / | / | / | CH₃ |
| Het-R.150 | Het.15 | H | / | / | H | / | H |
| Het-R.151 | Het.15 | CH₃ | / | / | H | / | H |
| Het-R.152 | Het.15 | H | / | / | CH₃ | / | H |
| Het-R.153 | Het.15 | CF₃ | / | / | H | / | H |
| Het-R.154 | Het.15 | H | / | / | CF₃ | / | H |
| Het-R.155 | Het.15 | Cl | / | / | H | / | H |
| Het-R.156 | Het.15 | H | / | / | Cl | / | H |
| Het-R.157 | Het.15 | H | / | / | H | / | CH₃ |
| Het-R.158 | Het.15 | CH₃ | / | / | H | / | CH₃ |
| Het-R.159 | Het.15 | H | / | / | CH₃ | / | CH₃ |
| Het-R.160 | Het.15 | CF₃ | / | / | H | / | CH₃ |
| Het-R.161 | Het.15 | H | / | / | CF₃ | / | CH₃ |
| Het-R.162 | Het.15 | Cl | / | / | H | / | CH₃ |
| Het-R.163 | Het.15 | H | / | / | Cl | / | CH₃ |
| Het-R.164 | Het.16 | / | H | / | H | / | H |
| Het-R.165 | Het.16 | / | H | / | CH₃ | / | H |
| Het-R.166 | Het.16 | / | CH₃ | / | H | / | H |
| Het-R.167 | Het.16 | / | H | / | CF₃ | / | H |
| Het-R.168 | Het.16 | / | CF₃ | / | H | / | H |
| Het-R.169 | Het.16 | / | H | / | Cl | / | H |
| Het-R.170 | Het.16 | / | Cl | / | H | / | H |
| Het-R.171 | Het.16 | / | H | / | H | / | CH₃ |
| Het-R.172 | Het.16 | / | H | / | CH₃ | / | CH₃ |
| Het-R.173 | Het.16 | / | CH₃ | / | H | / | CH₃ |
| Het-R.174 | Het.16 | / | H | / | CF₃ | / | CH₃ |
| Het-R.175 | Het.16 | / | CF₃ | / | H | / | CH₃ |
| Het-R.176 | Het.16 | / | H | / | Cl | / | CH₃ |
| Het-R.177 | Het.16 | / | Cl | / | H | / | CH₃ |
| Het-R.178 | Het.17 | H | H | / | H | / | / |
| Het-R.179 | Het.17 | CH₃ | H | / | H | / | / |
| Het-R.180 | Het.17 | H | CH₃ | / | H | / | / |
| Het-R.181 | Het.17 | H | H | / | CH₃ | / | / |
| Het-R.182 | Het.17 | CF₃ | H | / | H | / | / |
| Het-R.183 | Het.17 | H | CF₃ | / | H | / | / |
| Het-R.184 | Het.17 | H | H | / | CF₃ | / | / |
| Het-R.185 | Het.17 | Cl | H | / | H | / | / |
| Het-R.186 | Het.17 | H | Cl | / | H | / | / |
| Het-R.187 | Het.17 | H | H | / | Cl | / | / |
| Het-R.188 | Het.18 | H | H | / | / | / | / |
| Het-R.189 | Het.18 | H | CH₃ | / | / | / | / |
| Het-R.190 | Het.18 | CH₃ | H | / | / | / | / |
| Het-R.191 | Het.18 | H | CF₃ | / | / | / | / |
| Het-R.192 | Het.18 | CF₃ | H | / | / | / | / |
| Het-R.193 | Het.18 | H | Cl | / | / | / | / |
| Het-R.194 | Het.18 | Cl | H | / | / | / | / |
| Het-R.195 | Het.19 | H | / | H | / | / | / |
| Het-R.196 | Het.19 | CH₃ | / | H | / | / | / |
| Het-R.197 | Het.19 | H | / | CH₃ | / | / | / |
| Het-R.198 | Het.19 | CF₃ | / | H | / | / | / |
| Het-R.199 | Het.19 | H | / | CF₃ | / | / | / |
| Het-R.200 | Het.19 | Cl | / | H | / | / | / |
| Het-R.201 | Het.19 | H | / | Cl | / | / | / |
| Het-R.202 | Het.20 | / | H | H | / | / | / |
| Het-R.203 | Het.20 | / | CH₃ | H | / | / | / |
| Het-R.204 | Het.20 | / | H | CH₃ | / | / | / |
| Het-R.205 | Het.20 | / | CF₃ | H | / | / | / |
| Het-R.206 | Het.20 | / | H | CF₃ | / | / | / |
| Het-R.207 | Het.20 | / | Cl | H | / | / | / |
| Het-R.208 | Het.20 | / | H | Cl | / | / | / |
| Het-R.209 | Het.21 | H | H | / | / | / | / |
| Het-R.210 | Het.21 | H | CH₃ | / | / | / | / |
| Het-R.211 | Het.21 | CH₃ | H | / | / | / | / |
| Het-R.212 | Het.21 | H | CF₃ | / | / | / | / |
| Het-R.213 | Het.21 | CF₃ | H | / | / | / | / |
| Het-R.214 | Het.21 | H | Cl | / | / | / | / |
| Het-R.215 | Het.21 | Cl | H | / | / | / | / |
| Het-R.216 | Het.22 | H | / | H | / | / | / |
| Het-R.217 | Het.22 | CH₃ | / | H | / | / | / |
| Het-R.218 | Het.22 | H | / | CH₃ | / | / | / |
| Het-R.219 | Het.22 | CF₃ | / | H | / | / | / |
| Het-R.220 | Het.22 | H | / | CF₃ | / | / | / |
| Het-R.221 | Het.22 | Cl | / | H | / | / | / |
| Het-R.222 | Het.22 | H | / | Cl | / | / | / |
| Het-R.223 | Het.23 | / | H | H | / | / | / |
| Het-R.224 | Het.23 | / | CH₃ | H | / | / | / |
| Het-R.225 | Het.23 | / | H | CH₃ | / | / | / |
| Het-R.226 | Het.23 | / | CF₃ | H | / | / | / |
| Het-R.227 | Het.23 | / | H | CF₃ | / | / | / |
| Het-R.228 | Het.23 | / | Cl | H | / | / | / |
| Het-R.229 | Het.23 | / | H | Cl | / | / | / |
| Het-R.230 | Het.24 | H | H | H | / | / | / |
| Het-R.231 | Het.24 | CH₃ | H | H | / | / | / |
| Het-R.232 | Het.24 | H | CH₃ | H | / | / | / |
| Het-R.233 | Het.24 | H | H | CH₃ | / | / | / |
| Het-R.234 | Het.24 | CF₃ | H | H | / | / | / |
| Het-R.235 | Het.24 | H | CF₃ | H | / | / | / |
| Het-R.236 | Het.24 | H | H | CF₃ | / | / | / |
| Het-R.237 | Het.24 | Cl | H | H | / | / | / |
| Het-R.238 | Het.24 | H | Cl | H | / | / | / |
| Het-R.239 | Het.24 | H | H | Cl | / | / | / |
| Het-R.240 | Het.25 | H | H | / | / | / | H |
| Het-R.241 | Het.25 | H | CH₃ | / | / | / | H |
| Het-R.242 | Het.25 | CH₃ | H | / | / | / | H |
| Het-R.243 | Het.25 | H | CF₃ | / | / | / | H |
| Het-R.244 | Het.25 | CF₃ | H | / | / | / | H |
| Het-R.245 | Het.25 | H | Cl | / | / | / | H |
| Het-R.246 | Het.25 | Cl | H | / | / | / | H |
| Het-R.247 | Het.25 | H | H | / | / | / | CH₃ |
| Het-R.248 | Het.25 | H | CH₃ | / | / | / | CH₃ |
| Het-R.249 | Het.25 | CH₃ | H | / | / | / | CH₃ |
| Het-R.250 | Het.25 | H | CF₃ | / | / | / | CH₃ |
| Het-R.251 | Het.25 | CF₃ | H | / | / | / | CH₃ |
| Het-R.252 | Het.25 | H | Cl | / | / | / | CH₃ |
| Het-R.253 | Het.25 | Cl | H | / | / | / | CH₃ |
| Het-R.254 | Het.26 | H | / | H | / | / | H |
| Het-R.255 | Het.26 | CH₃ | / | H | / | / | H |
| Het-R.256 | Het.26 | H | / | CH₃ | / | / | H |
| Het-R.257 | Het.26 | CF₃ | / | H | / | / | H |
| Het-R.258 | Het.26 | H | / | CF₃ | / | / | H |
| Het-R.259 | Het.26 | Cl | / | H | / | / | H |
| Het-R.260 | Het.26 | H | / | Cl | / | / | H |
| Het-R.261 | Het.26 | H | / | H | / | / | CH₃ |
| Het-R.262 | Het.26 | CH₃ | / | H | / | / | CH₃ |
| Het-R.263 | Het.26 | H | / | CH₃ | / | / | CH₃ |
| Het-R.264 | Het.26 | CF₃ | / | H | / | / | CH₃ |
| Het-R.265 | Het.26 | H | / | CF₃ | / | / | CH₃ |
| Het-R.266 | Het.26 | Cl | / | H | / | / | CH₃ |
| Het-R.267 | Het.26 | H | / | Cl | / | / | CH₃ |
| Het-R.268 | Het.27 | / | H | H | / | / | H |
| Het-R.269 | Het.27 | / | CH₃ | H | / | / | H |
| Het-R.270 | Het.27 | / | H | CH₃ | / | / | H |
| Het-R.271 | Het.27 | / | CF₃ | H | / | / | H |
| Het-R.272 | Het.27 | / | H | CF₃ | / | / | H |
| Het-R.273 | Het.27 | / | Cl | H | / | / | H |
| Het-R.274 | Het.27 | / | H | Cl | / | / | H |
| Het-R.275 | Het.27 | / | H | H | / | / | CH₃ |
| Het-R.276 | Het.27 | / | CH₃ | H | / | / | CH₃ |
| Het-R.277 | Het.27 | / | H | CH₃ | / | / | CH₃ |
| Het-R.278 | Het.27 | / | CF₃ | H | / | / | CH₃ |
| Het-R.279 | Het.27 | / | H | CF₃ | / | / | CH₃ |
| Het-R.280 | Het.27 | / | Cl | H | / | / | CH₃ |
| Het-R.281 | Het.27 | / | H | Cl | / | / | CH₃ |
| Het-R.282 | Het.28 | H | / | / | / | / | / |
| Het-R.283 | Het.28 | CH₃ | / | / | / | / | / |
| Het-R.284 | Het.28 | CF₃ | / | / | / | / | / |
| Het-R.285 | Het.28 | Cl | / | / | / | / | / |
| Het-R.286 | Het.29 | H | / | / | / | / | / |
| Het-R.287 | Het.29 | CH₃ | / | / | / | / | / |
| Het-R.288 | Het.29 | CF₃ | / | / | / | / | / |
| Het-R.289 | Het.29 | Cl | / | / | / | / | / |
| Het-R.290 | Het.30 | H | / | / | / | / | / |
| Het-R.291 | Het.30 | CH₃ | / | / | / | / | / |
| Het-R.292 | Het.30 | CF₃ | / | / | / | / | / |
| Het-R.293 | Het.30 | Cl | / | / | / | / | / |
| Het-R.294 | Het.31 | H | / | / | / | / | / |
| Het-R.295 | Het.31 | CH₃ | / | / | / | / | / |
| Het-R.296 | Het.31 | CF₃ | / | / | / | / | / |
| Het-R.297 | Het.31 | Cl | / | / | / | / | / |
| Het-R.298 | Het.32 | H | / | / | / | / | / |
| Het-R.299 | Het.32 | CH₃ | / | / | / | / | / |
| Het-R.300 | Het.32 | CF₃ | / | / | / | / | / |
| Het-R.301 | Het.32 | Cl | / | / | / | / | / |
| Het-R.302 | Het.33 | H | / | / | / | / | / |
| Het-R.303 | Het.33 | CH₃ | / | / | / | / | / |
| Het-R.304 | Het.33 | CF₃ | / | / | / | / | / |
| Het-R.305 | Het.33 | Cl | / | / | / | / | / |
| Het-R.306 | Het.34 | H | H | / | / | / | / |
| Het-R.307 | Het.34 | H | CH₃ | / | / | / | / |
| Het-R.308 | Het.34 | CH₃ | H | / | / | / | / |
| Het-R.309 | Het.34 | H | CF₃ | / | / | / | / |
| Het-R.310 | Het.34 | CF₃ | H | / | / | / | / |
| Het-R.311 | Het.34 | H | Cl | / | / | / | / |
| Het-R.312 | Het.34 | Cl | H | / | / | / | / |
| Het-R.313 | Het.35 | H | / | / | / | / | H |
| Het-R.314 | Het.35 | CH₃ | / | / | / | / | H |
| Het-R.315 | Het.35 | CF₃ | / | / | / | / | H |
| Het-R.316 | Het.35 | Cl | / | / | / | / | H |
| Het-R.317 | Het.35 | H | / | / | / | / | CH₃ |
| Het-R.318 | Het.35 | CH₃ | / | / | / | / | CH₃ |
| Het-R.319 | Het.35 | CF₃ | / | / | / | / | CH₃ |
| Het-R.320 | Het.35 | Cl | / | / | / | / | CH₃ |
| Het-R.321 | Het.36 | H | / | / | / | / | H |
| Het-R.322 | Het.36 | CH₃ | / | / | / | / | H |
| Het-R.323 | Het.36 | CF₃ | / | / | / | / | H |
| Het-R.324 | Het.36 | Cl | / | / | / | / | H |
| Het-R.325 | Het.36 | H | / | / | / | / | CH₃ |
| Het-R.326 | Het.36 | CH₃ | / | / | / | / | CH₃ |
| Het-R.327 | Het.36 | CF₃ | / | / | / | / | CH₃ |
| Het-R.328 | Het.36 | Cl | / | / | / | / | CH₃ |
| Het-R.329 | Het.37 | H | / | / | / | / | H |
| Het-R.330 | Het.37 | CH₃ | / | / | / | / | H |
| Het-R.331 | Het.37 | CF₃ | / | / | / | / | H |
| Het-R.332 | Het.37 | Cl | / | / | / | / | H |
| Het-R.333 | Het.37 | H | / | / | / | / | CH₃ |
| Het-R.334 | Het.37 | CH₃ | / | / | / | / | CH₃ |
| Het-R.335 | Het.37 | CF₃ | / | / | / | / | CH₃ |
| Het-R.336 | Het.37 | Cl | / | / | / | / | CH₃ |
| Het-R.337 | Het.38 | H | H | / | / | / | / |
| Het-R.338 | Het.38 | H | CH₃ | / | / | / | / |
| Het-R.339 | Het.38 | CH₃ | H | / | / | / | / |
| Het-R.340 | Het.38 | H | CF₃ | / | / | / | / |
| Het-R.341 | Het.38 | CF₃ | H | / | / | / | / |
| Het-R.342 | Het.38 | H | Cl | / | / | / | / |
| Het-R.343 | Het.38 | Cl | H | / | / | / | / |
| Het-R.344 | Het.39 | H | / | / | / | / | H |
| Het-R.345 | Het.39 | CH₃ | / | / | / | / | H |
| Het-R.346 | Het.39 | CF₃ | / | / | / | / | H |
| Het-R.347 | Het.39 | Cl | / | / | / | / | H |
| Het-R.348 | Het.39 | H | / | / | / | / | CH₃ |
| Het-R.349 | Het.39 | CH₃ | / | / | / | / | CH₃ |
| Het-R.350 | Het.39 | CF₃ | / | / | / | / | CH₃ |
| Het-R.351 | Het.39 | Cl | / | / | / | / | CH₃ |
| Het-R.352 | Het.40 | / | H | / | / | / | H |
| Het-R.353 | Het.40 | / | CH₃ | / | / | / | H |
| Het-R.354 | Het.40 | / | CF₃ | / | / | / | H |
| Het-R.355 | Het.40 | / | Cl | / | / | / | H |
| Het-R.356 | Het.40 | / | H | / | / | / | CH₃ |
| Het-R.357 | Het.40 | / | CH₃ | / | / | / | CH₃ |
| Het-R.358 | Het.40 | / | CF₃ | / | / | / | CH₃ |
| Het-R.359 | Het.40 | / | Cl | / | / | / | CH₃ |
| Het-R.360 | Het.41 | H | / | / | / | / | / |
| Het-R.361 | Het.41 | CH₃ | / | / | / | / | / |
| Het-R.362 | Het.41 | CF₃ | / | / | / | / | / |
| Het-R.363 | Het.41 | Cl | / | / | / | / | / |
| Het-R.364 | Het.42 | / | / | / | / | / | H |
| Het-R.365 | Het.42 | / | / | / | / | / | CH₃ |
| Het-R.366 | Het.43 | H | / | / | / | / | / |
| Het-R.367 | Het.43 | CH₃ | / | / | / | / | / |
| Het-R.368 | Het.43 | CF₃ | / | / | / | / | / |
| Het-R.369 | Het.43 | Cl | / | / | / | / | / |
| Het-R.370 | Het.44 | / | H | / | / | / | / |
| Het-R.371 | Het.44 | / | CH₃ | / | / | / | / |
| Het-R.372 | Het.44 | / | CF₃ | / | / | / | / |
| Het-R.373 | Het.44 | / | Cl | / | / | / | / |
| Het-R.374 | Het.45 | H | / | / | / | / | / |
| Het-R.375 | Het.45 | CH₃ | / | / | / | / | / |
| Het-R.376 | Het.45 | CF₃ | / | / | / | / | / |
| Het-R.377 | Het.45 | Cl | / | / | / | / | / |
| Het-R.378 | Het.46 | / | H | / | / | / | / |
| Het-R.379 | Het.46 | / | CH₃ | / | / | / | / |
| Het-R.380 | Het.46 | / | CF₃ | / | / | / | / |
| Het-R.381 | Het.46 | / | Cl | / | / | / | / |
| Het-R.382 | Het.47 | H | H | H | H | / | / |
| Het-R.383 | Het.47 | CH₃ | H | H | H | / | / |
| Het-R.384 | Het.47 | H | CH₃ | H | H | / | / |
| Het-R.385 | Het.47 | H | H | CH₃ | H | / | / |
| Het-R.386 | Het.47 | H | H | H | CH₃ | / | / |
| Het-R.387 | Het.47 | CF₃ | H | H | H | / | / |
| Het-R.388 | Het.47 | H | CF₃ | H | H | / | / |
| Het-R.389 | Het.47 | H | H | CF₃ | H | / | / |
| Het-R.390 | Het.47 | H | H | H | CF₃ | / | / |
| Het-R.391 | Het.47 | Cl | H | H | H | / | / |
| Het-R.392 | Het.47 | H | Cl | H | H | / | / |
| Het-R.393 | Het.47 | H | H | Cl | H | / | / |
| Het-R.394 | Het.47 | H | H | H | Cl | / | / |
| Het-R.395 | Het.48 | H | H | H | / | H | / |
| Het-R.396 | Het.48 | CH₃ | H | H | / | H | / |
| Het-R.397 | Het.48 | H | CH₃ | H | / | H | / |
| Het-R.398 | Het.48 | H | H | CH₃ | / | H | / |
| Het-R.399 | Het.48 | H | H | H | / | CH₃ | / |
| Het-R.400 | Het.48 | CF₃ | H | H | / | H | / |
| Het-R.401 | Het.48 | H | CF₃ | H | / | H | / |
| Het-R.402 | Het.48 | H | H | CF₃ | / | H | / |
| Het-R.403 | Het.48 | H | H | H | / | CF₃ | / |
| Het-R.404 | Het.48 | Cl | H | H | / | H | / |
| Het-R.405 | Het.48 | H | Cl | H | / | H | / |
| Het-R.406 | Het.48 | H | H | Cl | / | H | / |
| Het-R.407 | Het.48 | H | H | H | / | Cl | / |
| Het-R.408 | Het.49 | H | H | / | H | H | / |
| Het-R.409 | Het.49 | CH₃ | H | / | H | H | / |
| Het-R.410 | Het.49 | H | CH₃ | / | H | H | / |
| Het-R.411 | Het.49 | H | H | / | CH₃ | H | / |
| Het-R.412 | Het.49 | H | H | / | H | CH₃ | / |
| Het-R.413 | Het.49 | CF₃ | H | / | H | H | / |
| Het-R.414 | Het.49 | H | CF₃ | / | H | H | / |
| Het-R.415 | Het.49 | H | H | / | CF₃ | H | / |
| Het-R.416 | Het.49 | H | H | / | H | CF₃ | / |
| Het-R.417 | Het.49 | Cl | H | / | H | H | / |
| Het-R.418 | Het.49 | H | Cl | / | H | H | / |
| Het-R.419 | Het.49 | H | H | / | Cl | H | / |
| Het-R.420 | Het.49 | H | H | / | H | Cl | / |
| Het-R.421 | Het.50 | H | H | H | / | / | / |
| Het-R.422 | Het.50 | CH₃ | H | H | / | / | / |
| Het-R.423 | Het.50 | H | CH₃ | H | / | / | / |
| Het-R.424 | Het.50 | H | H | CH₃ | / | / | / |
| Het-R.425 | Het.50 | CF₃ | H | H | / | / | / |
| Het-R.426 | Het.50 | H | CF₃ | H | / | / | / |
| Het-R.427 | Het.50 | H | H | CF₃ | / | / | / |
| Het-R.428 | Het.50 | Cl | H | H | / | / | / |
| Het-R.429 | Het.50 | H | Cl | H | / | / | / |
| Het-R.430 | Het.50 | H | H | Cl | / | / | / |
| Het-R.431 | Het.51 | H | H | / | H | / | / |
| Het-R.432 | Het.51 | CH₃ | H | / | H | / | / |
| Het-R.433 | Het.51 | H | CH₃ | / | H | / | / |
| Het-R.434 | Het.51 | H | H | / | CH₃ | / | / |
| Het-R.435 | Het.51 | CF₃ | H | / | H | / | / |
| Het-R.436 | Het.51 | H | CF₃ | / | H | / | / |
| Het-R.437 | Het.51 | H | H | / | CF₃ | / | / |
| Het-R.438 | Het.51 | Cl | H | / | H | / | / |
| Het-R.439 | Het.51 | H | Cl | / | H | / | / |
| Het-R.440 | Het.51 | H | H | / | Cl | / | / |
| Het-R.441 | Het.52 | H | H | H | / | / | / |
| Het-R.442 | Het.52 | CH₃ | H | H | / | / | / |
| Het-R.443 | Het.52 | H | CH₃ | H | / | / | / |
| Het-R.444 | Het.52 | H | H | CH₃ | / | / | / |
| Het-R.445 | Het.52 | CF₃ | H | H | / | / | / |
| Het-R.446 | Het.52 | H | CF₃ | H | / | / | / |
| Het-R.447 | Het.52 | H | H | CF₃ | / | / | / |
| Het-R.448 | Het.52 | Cl | H | H | / | / | / |
| Het-R.449 | Het.52 | H | Cl | H | / | / | / |
| Het-R.450 | Het.52 | H | H | Cl | / | / | / |
| Het-R.451 | Het.53 | H | H | / | / | H | / |
| Het-R.452 | Het.53 | CH₃ | H | / | / | H | / |
| Het-R.453 | Het.53 | H | CH₃ | / | / | H | / |
| Het-R.454 | Het.53 | H | H | / | / | CH₃ | / |
| Het-R.455 | Het.53 | CF₃ | H | / | / | H | / |
| Het-R.456 | Het.53 | H | CF₃ | / | / | H | / |
| Het-R.457 | Het.53 | H | H | / | / | CF₃ | / |
| Het-R.458 | Het.53 | Cl | H | / | / | H | / |
| Het-R.459 | Het.53 | H | Cl | / | / | H | / |
| Het-R.460 | Het.53 | H | H | / | / | Cl | / |
| Het-R.461 | Het.54 | H | / | H | / | H | / |
| Het-R.462 | Het.54 | CH₃ | / | H | / | H | / |
| Het-R.463 | Het.54 | H | / | CH₃ | / | H | / |
| Het-R.464 | Het.54 | H | / | H | / | CH₃ | / |
| Het-R.465 | Het.54 | CF₃ | / | H | / | H | / |
| Het-R.466 | Het.54 | H | / | CF₃ | / | H | / |
| Het-R.467 | Het.54 | H | / | H | / | CF₃ | / |
| Het-R.468 | Het.54 | Cl | / | H | / | H | / |
| Het-R.469 | Het.54 | H | / | Cl | / | H | / |
| Het-R.470 | Het.54 | H | / | H | / | Cl | / |
| Het-R.471 | Het.55 | H | H | / | H | / | / |
| Het-R.472 | Het.55 | CH₃ | H | / | H | / | / |
| Het-R.473 | Het.55 | H | CH₃ | / | H | / | / |
| Het-R.474 | Het.55 | H | H | / | CH₃ | / | / |
| Het-R.475 | Het.55 | CF₃ | H | / | H | / | / |
| Het-R.476 | Het.55 | H | CF₃ | / | H | / | / |
| Het-R.477 | Het.55 | H | H | / | CF₃ | / | / |
| Het-R.478 | Het.55 | Cl | H | / | H | / | / |
| Het-R.479 | Het.55 | H | Cl | / | H | / | / |
| Het-R.480 | Het.55 | H | H | / | Cl | / | / |
| Het-R.481 | Het.56 | H | / | H | / | / | / |
| Het-R.482 | Het.56 | CH₃ | / | H | / | / | / |
| Het-R.483 | Het.56 | H | / | CH₃ | / | / | / |
| Het-R.484 | Het.56 | CF₃ | / | H | / | / | / |
| Het-R.485 | Het.56 | H | / | CF₃ | / | / | / |
| Het-R.486 | Het.56 | Cl | / | H | / | / | / |
| Het-R.487 | Het.56 | H | / | Cl | / | / | / |
| Het-R.488 | Het.57 | H | H | / | / | / | / |
| Het-R.489 | Het.57 | H | CH₃ | / | / | / | / |
| Het-R.490 | Het.57 | CH₃ | H | / | / | / | / |
| Het-R.491 | Het.57 | H | CF₃ | / | / | / | / |
| Het-R.492 | Het.57 | CF₃ | H | / | / | / | / |
| Het-R.493 | Het.57 | H | Cl | / | / | / | / |
| Het-R.494 | Het.57 | Cl | H | / | / | / | / |
| Het-R.495 | Het.58 | H | / | / | / | H | / |
| Het-R.496 | Het.58 | CH₃ | / | / | / | H | / |
| Het-R.497 | Het.58 | H | / | / | / | CH₃ | / |
| Het-R.498 | Het.58 | CF₃ | / | / | / | H | / |
| Het-R.499 | Het.58 | H | / | / | / | CF₃ | / |
| Het-R.500 | Het.58 | Cl | / | / | / | H | / |
| Het-R.501 | Het.58 | H | / | / | / | Cl | / |
| Het-R.502 | Het.59 | / | H | / | / | H | / |
| Het-R.503 | Het.59 | / | CH₃ | / | / | H | / |
| Het-R.504 | Het.59 | / | H | / | / | CH₃ | / |
| Het-R.505 | Het.59 | / | CF₃ | / | / | H | / |
| Het-R.506 | Het.59 | / | H | / | / | CF₃ | / |
| Het-R.507 | Het.59 | / | Cl | / | / | H | / |
| Het-R.508 | Het.59 | / | H | / | / | Cl | / |
| Het-R.509 | Het.60 | H | / | / | / | / | / |
| Het-R.510 | Het.60 | CH₃ | / | / | / | / | / |
| Het-R.511 | Het.60 | CF₃ | / | / | / | / | / |
| Het-R.512 | Het.60 | Cl | / | / | / | / | / |

Examples for such preferred compounds are given in tables 1 to 36.

Table 1: Compounds of the formulae Ia or Ib and their mixtures, wherein X is O, A is CH₂, R^{2a} or R^{2b} is hydrogen, R^{3a}, R^{3b}, R^{3c} and R^{3d} are hydrogen and wherein R¹ and Ar have the meanings given in each line of table B. These compounds are also referred to as Ia-A-1 to Ia-A-3600 or Ib-A-1 to Ib-B-3600).

TABLE B

| | R¹ | Ar |
|---|---|---|
| A-1 | CN | Ar.0 |
| A-2 | OCH₃ | Ar.0 |
| A-3 | SCH₃ | Ar.0 |
| A-4 | OCF₃ | Ar.0 |
| A-5 | OCHF₂ | Ar.0 |
| A-6 | methyl | Ar.0 |
| A-7 | iso-propyl | Ar.0 |
| A-8 | ethenyl | Ar.0 |
| A-9 | ethynyl | Ar.0 |
| A-10 | Ar.0 | Ar.0 |
| A-11 | Ar.1 | Ar.0 |
| A-12 | Ar.2 | Ar.0 |
| A-13 | Ar.3 | Ar.0 |
| A-14 | Ar.4 | Ar.0 |
| A-15 | Ar.5 | Ar.0 |
| A-16 | Ar.6 | Ar.0 |
| A-17 | Ar.7 | Ar.0 |
| A-18 | Ar.8 | Ar.0 |
| A-19 | Ar.9 | Ar.0 |
| A-20 | Ar.10 | Ar.0 |
| A-21 | Ar.11 | Ar.0 |
| A-22 | Ar.12 | Ar.0 |
| A-23 | Ar.13 | Ar.0 |
| A-24 | Ar.14 | Ar.0 |
| A-25 | Ar.15 | Ar.0 |
| A-26 | Ar.16 | Ar.0 |
| A-27 | Ar.17 | Ar.0 |
| A-28 | Ar.18 | Ar.0 |
| A-29 | Ar.19 | Ar.0 |
| A-30 | Ar.20 | Ar.0 |
| A-31 | Ar.21 | Ar.0 |
| A-32 | Ar.22 | Ar.0 |
| A-33 | Ar.23 | Ar.0 |
| A-34 | Ar.24 | Ar.0 |
| A-35 | Ar.25 | Ar.0 |
| A-36 | Ar.26 | Ar.0 |
| A-37 | Ar.27 | Ar.0 |
| A-38 | Ar.28 | Ar.0 |
| A-39 | Ar.29 | Ar.0 |
| A-40 | Ar.30 | Ar.0 |
| A-41 | Ar.31 | Ar.0 |
| A-42 | Ar.32 | Ar.0 |
| A-43 | Ar.33 | Ar.0 |
| A-44 | Ar.34 | Ar.0 |
| A-45 | Ar.35 | Ar.0 |
| A-46 | Ar.36 | Ar.0 |
| A-47 | Ar.37 | Ar.0 |
| A-48 | Ar.38 | Ar.0 |
| A-49 | Ar.39 | Ar.0 |
| A-50 | Ar.40 | Ar.0 |
| A-51 | Ar.41 | Ar.0 |
| A-52 | Ar.42 | Ar.0 |
| A-53 | Ar.43 | Ar.0 |
| A-54 | Ar.44 | Ar.0 |
| A-55 | Ar.45 | Ar.0 |
| A-56 | Ar.46 | Ar.0 |
| A-57 | Ar.47 | Ar.0 |
| A-58 | Ar.48 | Ar.0 |
| A-59 | Ar.49 | Ar.0 |
| A-60 | Ar.50 | Ar.0 |
| A-61 | Ar.51 | Ar.0 |
| A-62 | Ar.52 | Ar.0 |
| A-63 | Ar.53 | Ar.0 |
| A-64 | Ar.54 | Ar.0 |
| A-65 | Ar.55 | Ar.0 |
| A-66 | Ar.56 | Ar.0 |
| A-67 | Ar.57 | Ar.0 |
| A-68 | Ar.58 | Ar.0 |
| A-69 | Ar.59 | Ar.0 |
| A-70 | Ar.60 | Ar.0 |
| A-71 | Ar.61 | Ar.0 |
| A-72 | Ar.62 | Ar.0 |
| A-73 | Ar.63 | Ar.0 |
| A-74 | Ar.64 | Ar.0 |
| A-75 | Ar.65 | Ar.0 |
| A-76 | Ar.66 | Ar.0 |
| A-77 | Ar.67 | Ar.0 |
| A-78 | Ar.68 | Ar.0 |
| A-79 | Ar.69 | Ar.0 |
| A-80 | Ar.70 | Ar.0 |
| A-81 | Ar.71 | Ar.0 |
| A-82 | Ar.72 | Ar.0 |
| A-83 | Ar.73 | Ar.0 |
| A-84 | Ar.74 | Ar.0 |
| A-85 | Ar.75 | Ar.0 |
| A-86 | Ar.76 | Ar.0 |
| A-87 | Ar.77 | Ar.0 |
| A-88 | Ar.78 | Ar.0 |
| A-89 | Ar.79 | Ar.0 |
| A-90 | Ar.80 | Ar.0 |
| A-91 | CN | Ar.1 |
| A-92 | OCH₃ | Ar.1 |
| A-93 | SCH₃ | Ar.1 |
| A-94 | OCF₃ | Ar.1 |
| A-95 | OCHF₂ | Ar.1 |
| A-96 | methyl | Ar.1 |
| A-97 | iso-propyl | Ar.1 |
| A-98 | ethenyl | Ar.1 |
| A-99 | ethynyl | Ar.1 |
| A-100 | Ar.0 | Ar.1 |
| A-101 | Ar.1 | Ar.1 |
| A-102 | Ar.2 | Ar.1 |
| A-103 | Ar.3 | Ar.1 |
| A-104 | Ar.4 | Ar.1 |
| A-105 | Ar.5 | Ar.1 |
| A-106 | Ar.6 | Ar.1 |
| A-107 | Ar.7 | Ar.1 |
| A-108 | Ar.8 | Ar.1 |
| A-109 | Ar.9 | Ar.1 |
| A-110 | Ar.10 | Ar.1 |
| A-111 | Ar.11 | Ar.1 |
| A-112 | Ar.12 | Ar.1 |
| A-113 | Ar.13 | Ar.1 |
| A-114 | Ar.14 | Ar.1 |
| A-115 | Ar.15 | Ar.1 |
| A-116 | Ar.16 | Ar.1 |
| A-117 | Ar.17 | Ar.1 |
| A-118 | Ar.18 | Ar.1 |
| A-119 | Ar.19 | Ar.1 |
| A-120 | Ar.20 | Ar.1 |
| A-121 | Ar.21 | Ar.1 |
| A-122 | Ar.22 | Ar.1 |
| A-123 | Ar.23 | Ar.1 |
| A-124 | Ar.24 | Ar.1 |
| A-125 | Ar.25 | Ar.1 |
| A-126 | Ar.26 | Ar.1 |
| A-127 | Ar.27 | Ar.1 |
| A-128 | Ar.28 | Ar.1 |
| A-129 | Ar.29 | Ar.1 |
| A-130 | Ar.30 | Ar.1 |
| A-131 | Ar.31 | Ar.1 |
| A-132 | Ar.32 | Ar.1 |
| A-133 | Ar.33 | Ar.1 |
| A-134 | Ar.34 | Ar.1 |
| A-135 | Ar.35 | Ar.1 |
| A-136 | Ar.36 | Ar.1 |
| A-137 | Ar.37 | Ar.1 |
| A-138 | Ar.38 | Ar.1 |
| A-139 | Ar.39 | Ar.1 |
| A-140 | Ar.40 | Ar.1 |
| A-141 | Ar.41 | Ar.1 |
| A-142 | Ar.42 | Ar.1 |
| A-143 | Ar.43 | Ar.1 |
| A-144 | Ar.44 | Ar.1 |
| A-145 | Ar.45 | Ar.1 |
| A-146 | Ar.46 | Ar.1 |
| A-147 | Ar.47 | Ar.1 |
| A-148 | Ar.48 | Ar.1 |
| A-149 | Ar.49 | Ar.1 |
| A-150 | Ar.50 | Ar.1 |
| A-151 | Ar.51 | Ar.1 |
| A-152 | Ar.52 | Ar.1 |
| A-153 | Ar.53 | Ar.1 |
| A-154 | Ar.54 | Ar.1 |
| A-155 | Ar.55 | Ar.1 |
| A-156 | Ar.56 | Ar.1 |
| A-157 | Ar.57 | Ar.1 |
| A-158 | Ar.58 | Ar.1 |
| A-159 | Ar.59 | Ar.1 |
| A-160 | Ar.60 | Ar.1 |
| A-161 | Ar.61 | Ar.1 |
| A-162 | Ar.62 | Ar.1 |
| A-163 | Ar.63 | Ar.1 |
| A-164 | Ar.64 | Ar.1 |
| A-165 | Ar.65 | Ar.1 |
| A-166 | Ar.66 | Ar.1 |
| A-167 | Ar.67 | Ar.1 |
| A-168 | Ar.68 | Ar.1 |
| A-169 | Ar.69 | Ar.1 |
| A-170 | Ar.70 | Ar.1 |
| A-171 | Ar.71 | Ar.1 |
| A-172 | Ar.72 | Ar.1 |
| A-173 | Ar.73 | Ar.1 |
| A-174 | Ar.74 | Ar.1 |
| A-175 | Ar.75 | Ar.1 |
| A-176 | Ar.76 | Ar.1 |
| A-177 | Ar.77 | Ar.1 |
| A-178 | Ar.78 | Ar.1 |
| A-179 | Ar.79 | Ar.1 |
| A-180 | Ar.80 | Ar.1 |
| A-181 | CN | Ar.2 |
| A-182 | OCH₃ | Ar.2 |
| A-183 | SCH₃ | Ar.2 |
| A-184 | OCF₃ | Ar.2 |
| A-185 | OCHF₂ | Ar.2 |
| A-186 | methyl | Ar.2 |
| A-187 | iso-propyl | Ar.2 |
| A-188 | ethenyl | Ar.2 |
| A-189 | ethynyl | Ar.2 |
| A-190 | Ar.0 | Ar.2 |
| A-191 | Ar.1 | Ar.2 |
| A-192 | Ar.2 | Ar.2 |
| A-193 | Ar.3 | Ar.2 |
| A-194 | Ar.4 | Ar.2 |
| A-195 | Ar.5 | Ar.2 |
| A-196 | Ar.6 | Ar.2 |
| A-197 | Ar.7 | Ar.2 |
| A-198 | Ar.8 | Ar.2 |
| A-199 | Ar.9 | Ar.2 |
| A-200 | Ar.10 | Ar.2 |
| A-201 | Ar.11 | Ar.2 |
| A-202 | Ar.12 | Ar.2 |
| A-203 | Ar.13 | Ar.2 |
| A-204 | Ar.14 | Ar.2 |
| A-205 | Ar.15 | Ar.2 |
| A-206 | Ar.16 | Ar.2 |
| A-207 | Ar.17 | Ar.2 |
| A-208 | Ar.18 | Ar.2 |
| A-209 | Ar.19 | Ar.2 |
| A-210 | Ar.20 | Ar.2 |
| A-211 | Ar.21 | Ar.2 |
| A-212 | Ar.22 | Ar.2 |
| A-213 | Ar.23 | Ar.2 |
| A-214 | Ar.24 | Ar.2 |
| A-215 | Ar.25 | Ar.2 |
| A-216 | Ar.26 | Ar.2 |
| A-217 | Ar.27 | Ar.2 |
| A-218 | Ar.28 | Ar.2 |
| A-219 | Ar.29 | Ar.2 |
| A-220 | Ar.30 | Ar.2 |
| A-221 | Ar.31 | Ar.2 |
| A-222 | Ar.32 | Ar.2 |
| A-223 | Ar.33 | Ar.2 |
| A-224 | Ar.34 | Ar.2 |
| A-225 | Ar.35 | Ar.2 |
| A-226 | Ar.36 | Ar.2 |
| A-227 | Ar.37 | Ar.2 |
| A-228 | Ar.38 | Ar.2 |
| A-229 | Ar.39 | Ar.2 |
| A-230 | Ar.40 | Ar.2 |
| A-231 | Ar.41 | Ar.2 |
| A-232 | Ar.42 | Ar.2 |
| A-233 | Ar.43 | Ar.2 |
| A-234 | Ar.44 | Ar.2 |
| A-235 | Ar.45 | Ar.2 |
| A-236 | Ar.46 | Ar.2 |
| A-237 | Ar.47 | Ar.2 |
| A-238 | Ar.48 | Ar.2 |
| A-239 | Ar.49 | Ar.2 |
| A-240 | Ar.50 | Ar.2 |
| A-241 | Ar.51 | Ar.2 |
| A-242 | Ar.52 | Ar.2 |
| A-243 | Ar.53 | Ar.2 |
| A-244 | Ar.54 | Ar.2 |
| A-245 | Ar.55 | Ar.2 |
| A-246 | Ar.56 | Ar.2 |
| A-247 | Ar.57 | Ar.2 |
| A-248 | Ar.58 | Ar.2 |
| A-249 | Ar.59 | Ar.2 |
| A-250 | Ar.60 | Ar.2 |
| A-251 | Ar.61 | Ar.2 |
| A-252 | Ar.62 | Ar.2 |
| A-253 | Ar.63 | Ar.2 |
| A-254 | Ar.64 | Ar.2 |
| A-255 | Ar.65 | Ar.2 |
| A-256 | Ar.66 | Ar.2 |
| A-257 | Ar.67 | Ar.2 |
| A-258 | Ar.68 | Ar.2 |
| A-259 | Ar.69 | Ar.2 |
| A-260 | Ar.70 | Ar.2 |
| A-261 | Ar.71 | Ar.2 |
| A-262 | Ar.72 | Ar.2 |
| A-263 | Ar.73 | Ar.2 |
| A-264 | Ar.74 | Ar.2 |
| A-265 | Ar.75 | Ar.2 |
| A-266 | Ar.76 | Ar.2 |
| A-267 | Ar.77 | Ar.2 |
| A-268 | Ar.78 | Ar.2 |
| A-269 | Ar.79 | Ar.2 |
| A-270 | Ar.80 | Ar.2 |
| A-271 | CN | Ar.3 |
| A-272 | OCH₃ | Ar.3 |
| A-273 | SCH₃ | Ar.3 |
| A-274 | OCF₃ | Ar.3 |
| A-275 | OCHF₂ | Ar.3 |
| A-276 | methyl | Ar.3 |
| A-277 | iso-propyl | Ar.3 |
| A-278 | ethenyl | Ar.3 |
| A-279 | ethynyl | Ar.3 |
| A-280 | Ar.0 | Ar.3 |
| A-281 | Ar.1 | Ar.3 |
| A-282 | Ar.2 | Ar.3 |
| A-283 | Ar.3 | Ar.3 |
| A-284 | Ar.4 | Ar.3 |
| A-285 | Ar.5 | Ar.3 |
| A-286 | Ar.6 | Ar.3 |
| A-287 | Ar.7 | Ar.3 |
| A-288 | Ar.8 | Ar.3 |
| A-289 | Ar.9 | Ar.3 |
| A-290 | Ar.10 | Ar.3 |
| A-291 | Ar.11 | Ar.3 |
| A-292 | Ar.12 | Ar.3 |
| A-293 | Ar.13 | Ar.3 |
| A-294 | Ar.14 | Ar.3 |
| A-295 | Ar.15 | Ar.3 |
| A-296 | Ar.16 | Ar.3 |
| A-297 | Ar.17 | Ar.3 |
| A-298 | Ar.18 | Ar.3 |
| A-299 | Ar.19 | Ar.3 |
| A-300 | Ar.20 | Ar.3 |
| A-301 | Ar.21 | Ar.3 |
| A-302 | Ar.22 | Ar.3 |
| A-303 | Ar.23 | Ar.3 |
| A-304 | Ar.24 | Ar.3 |
| A-305 | Ar.25 | Ar.3 |
| A-306 | Ar.26 | Ar.3 |
| A-307 | Ar.27 | Ar.3 |
| A-308 | Ar.28 | Ar.3 |
| A-309 | Ar.29 | Ar.3 |
| A-310 | Ar.30 | Ar.3 |
| A-311 | Ar.31 | Ar.3 |
| A-312 | Ar.32 | Ar.3 |
| A-313 | Ar.33 | Ar.3 |
| A-314 | Ar.34 | Ar.3 |
| A-315 | Ar.35 | Ar.3 |
| A-316 | Ar.36 | Ar.3 |
| A-317 | Ar.37 | Ar.3 |
| A-318 | Ar.38 | Ar.3 |
| A-319 | Ar.39 | Ar.3 |
| A-320 | Ar.40 | Ar.3 |
| A-321 | Ar.41 | Ar.3 |
| A-322 | Ar.42 | Ar.3 |
| A-323 | Ar.43 | Ar.3 |
| A-324 | Ar.44 | Ar.3 |
| A-325 | Ar.45 | Ar.3 |
| A-326 | Ar.46 | Ar.3 |
| A-327 | Ar.47 | Ar.3 |
| A-328 | Ar.48 | Ar.3 |
| A-329 | Ar.49 | Ar.3 |
| A-330 | Ar.50 | Ar.3 |
| A-331 | Ar.51 | Ar.3 |
| A-332 | Ar.52 | Ar.3 |
| A-333 | Ar.53 | Ar.3 |
| A-334 | Ar.54 | Ar.3 |
| A-335 | Ar.55 | Ar.3 |
| A-336 | Ar.56 | Ar.3 |
| A-337 | Ar.57 | Ar.3 |
| A-338 | Ar.58 | Ar.3 |
| A-339 | Ar.59 | Ar.3 |
| A-340 | Ar.60 | Ar.3 |
| A-341 | Ar.61 | Ar.3 |
| A-342 | Ar.62 | Ar.3 |
| A-343 | Ar.63 | Ar.3 |
| A-344 | Ar.64 | Ar.3 |
| A-345 | Ar.65 | Ar.3 |
| A-346 | Ar.66 | Ar.3 |
| A-347 | Ar.67 | Ar.3 |
| A-348 | Ar.68 | Ar.3 |
| A-349 | Ar.69 | Ar.3 |
| A-350 | Ar.70 | Ar.3 |
| A-351 | Ar.71 | Ar.3 |
| A-352 | Ar.72 | Ar.3 |
| A-353 | Ar.73 | Ar.3 |
| A-354 | Ar.74 | Ar.3 |
| A-355 | Ar.75 | Ar.3 |
| A-356 | Ar.76 | Ar.3 |
| A-357 | Ar.77 | Ar.3 |
| A-358 | Ar.78 | Ar.3 |
| A-359 | Ar.79 | Ar.3 |
| A-360 | Ar.80 | Ar.3 |
| A-361 | CN | Ar.4 |
| A-362 | OCH₃ | Ar.4 |
| A-363 | SCH₃ | Ar.4 |
| A-364 | OCF₃ | Ar.4 |
| A-365 | OCHF₂ | Ar.4 |
| A-366 | methyl | Ar.4 |
| A-367 | iso-propyl | Ar.4 |
| A-368 | ethenyl | Ar.4 |
| A-369 | ethynyl | Ar.4 |
| A-370 | Ar.0 | Ar.4 |
| A-371 | Ar.1 | Ar.4 |
| A-372 | Ar.2 | Ar.4 |
| A-373 | Ar.3 | Ar.4 |
| A-374 | Ar.4 | Ar.4 |
| A-375 | Ar.5 | Ar.4 |
| A-376 | Ar.6 | Ar.4 |
| A-377 | Ar.7 | Ar.4 |
| A-378 | Ar.8 | Ar.4 |
| A-379 | Ar.9 | Ar.4 |
| A-380 | Ar.10 | Ar.4 |
| A-381 | Ar.11 | Ar.4 |
| A-382 | Ar.12 | Ar.4 |
| A-383 | Ar.13 | Ar.4 |
| A-384 | Ar.14 | Ar.4 |
| A-385 | Ar.15 | Ar.4 |
| A-386 | Ar.16 | Ar.4 |
| A-387 | Ar.17 | Ar.4 |
| A-388 | Ar.18 | Ar.4 |
| A-389 | Ar.19 | Ar.4 |
| A-390 | Ar.20 | Ar.4 |
| A-391 | Ar.21 | Ar.4 |
| A-392 | Ar.22 | Ar.4 |
| A-393 | Ar.23 | Ar.4 |
| A-394 | Ar.24 | Ar.4 |
| A-395 | Ar.25 | Ar.4 |
| A-396 | Ar.26 | Ar.4 |
| A-397 | Ar.27 | Ar.4 |
| A-398 | Ar.28 | Ar.4 |
| A-399 | Ar.29 | Ar.4 |
| A-400 | Ar.30 | Ar.4 |
| A-401 | Ar.31 | Ar.4 |
| A-402 | Ar.32 | Ar.4 |
| A-403 | Ar.33 | Ar.4 |
| A-404 | Ar.34 | Ar.4 |
| A-405 | Ar.35 | Ar.4 |
| A-406 | Ar.36 | Ar.4 |
| A-407 | Ar.37 | Ar.4 |
| A-408 | Ar.38 | Ar.4 |
| A-409 | Ar.39 | Ar.4 |
| A-410 | Ar.40 | Ar.4 |
| A-411 | Ar.41 | Ar.4 |
| A-412 | Ar.42 | Ar.4 |
| A-413 | Ar.43 | Ar.4 |
| A-414 | Ar.44 | Ar.4 |
| A-415 | Ar.45 | Ar.4 |
| A-416 | Ar.46 | Ar.4 |
| A-417 | Ar.47 | Ar.4 |
| A-418 | Ar.48 | Ar.4 |
| A-419 | Ar.49 | Ar.4 |
| A-420 | Ar.50 | Ar.4 |
| A-421 | Ar.51 | Ar.4 |
| A-422 | Ar.52 | Ar.4 |
| A-423 | Ar.53 | Ar.4 |
| A-424 | Ar.54 | Ar.4 |
| A-425 | Ar.55 | Ar.4 |
| A-426 | Ar.56 | Ar.4 |
| A-427 | Ar.57 | Ar.4 |
| A-428 | Ar.58 | Ar.4 |
| A-429 | Ar.59 | Ar.4 |
| A-430 | Ar.60 | Ar.4 |
| A-431 | Ar.61 | Ar.4 |
| A-432 | Ar.62 | Ar.4 |
| A-433 | Ar.63 | Ar.4 |
| A-434 | Ar.64 | Ar.4 |
| A-435 | Ar.65 | Ar.4 |
| A-436 | Ar.66 | Ar.4 |
| A-437 | Ar.67 | Ar.4 |
| A-438 | Ar.68 | Ar.4 |
| A-439 | Ar.69 | Ar.4 |
| A-440 | Ar.70 | Ar.4 |
| A-441 | Ar.71 | Ar.4 |
| A-442 | Ar.72 | Ar.4 |
| A-443 | Ar.73 | Ar.4 |
| A-444 | Ar.74 | Ar.4 |
| A-445 | Ar.75 | Ar.4 |
| A-446 | Ar.76 | Ar.4 |
| A-447 | Ar.77 | Ar.4 |
| A-448 | Ar.78 | Ar.4 |
| A-449 | Ar.79 | Ar.4 |
| A-450 | Ar.80 | Ar.4 |
| A-451 | CN | Ar.5 |
| A-452 | OCH₃ | Ar.5 |
| A-453 | SCH₃ | Ar.5 |
| A-454 | OCF₃ | Ar.5 |
| A-455 | OCHF₂ | Ar.5 |
| A-456 | methyl | Ar.5 |
| A-457 | iso-propyl | Ar.5 |
| A-458 | ethenyl | Ar.5 |
| A-459 | ethynyl | Ar.5 |
| A-460 | Ar.0 | Ar.5 |
| A-461 | Ar.1 | Ar.5 |
| A-462 | Ar.2 | Ar.5 |
| A-463 | Ar.3 | Ar.5 |
| A-464 | Ar.4 | Ar.5 |
| A-465 | Ar.5 | Ar.5 |
| A-466 | Ar.6 | Ar.5 |
| A-467 | Ar.7 | Ar.5 |
| A-468 | Ar.8 | Ar.5 |
| A-469 | Ar.9 | Ar.5 |
| A-470 | Ar.10 | Ar.5 |
| A-471 | Ar.11 | Ar.5 |
| A-472 | Ar.12 | Ar.5 |
| A-473 | Ar.13 | Ar.5 |
| A-474 | Ar.14 | Ar.5 |
| A-475 | Ar.15 | Ar.5 |
| A-476 | Ar.16 | Ar.5 |
| A-477 | Ar.17 | Ar.5 |
| A-478 | Ar.18 | Ar.5 |
| A-479 | Ar.19 | Ar.5 |
| A-480 | Ar.20 | Ar.5 |
| A-481 | Ar.21 | Ar.5 |
| A-482 | Ar.22 | Ar.5 |
| A-483 | Ar.23 | Ar.5 |
| A-484 | Ar.24 | Ar.5 |
| A-485 | Ar.25 | Ar.5 |
| A-486 | Ar.26 | Ar.5 |
| A-487 | Ar.27 | Ar.5 |
| A-488 | Ar.28 | Ar.5 |
| A-489 | Ar.29 | Ar.5 |
| A-490 | Ar.30 | Ar.5 |
| A-491 | Ar.31 | Ar.5 |
| A-492 | Ar.32 | Ar.5 |
| A-493 | Ar.33 | Ar.5 |
| A-494 | Ar.34 | Ar.5 |
| A-495 | Ar.35 | Ar.5 |
| A-496 | Ar.36 | Ar.5 |
| A-497 | Ar.37 | Ar.5 |
| A-498 | Ar.38 | Ar.5 |
| A-499 | Ar.39 | Ar.5 |
| A-500 | Ar.40 | Ar.5 |
| A-501 | Ar.41 | Ar.5 |
| A-502 | Ar.42 | Ar.5 |
| A-503 | Ar.43 | Ar.5 |
| A-504 | Ar.44 | Ar.5 |
| A-505 | Ar.45 | Ar.5 |
| A-506 | Ar.46 | Ar.5 |
| A-507 | Ar.47 | Ar.5 |
| A-508 | Ar.48 | Ar.5 |
| A-509 | Ar.49 | Ar.5 |
| A-510 | Ar.50 | Ar.5 |
| A-511 | Ar.51 | Ar.5 |
| A-512 | Ar.52 | Ar.5 |
| A-513 | Ar.53 | Ar.5 |
| A-514 | Ar.54 | Ar.5 |
| A-515 | Ar.55 | Ar.5 |
| A-516 | Ar.56 | Ar.5 |
| A-517 | Ar.57 | Ar.5 |
| A-518 | Ar.58 | Ar.5 |
| A-519 | Ar.59 | Ar.5 |
| A-520 | Ar.60 | Ar.5 |
| A-521 | Ar.61 | Ar.5 |
| A-522 | Ar.62 | Ar.5 |
| A-523 | Ar.63 | Ar.5 |
| A-524 | Ar.64 | Ar.5 |
| A-525 | Ar.65 | Ar.5 |
| A-526 | Ar.66 | Ar.5 |
| A-527 | Ar.67 | Ar.5 |
| A-528 | Ar.68 | Ar.5 |
| A-529 | Ar.69 | Ar.5 |
| A-530 | Ar.70 | Ar.5 |
| A-531 | Ar.71 | Ar.5 |
| A-532 | Ar.72 | Ar.5 |
| A-533 | Ar.73 | Ar.5 |
| A-534 | Ar.74 | Ar.5 |
| A-535 | Ar.75 | Ar.5 |
| A-536 | Ar.76 | Ar.5 |
| A-537 | Ar.77 | Ar.5 |
| A-538 | Ar.78 | Ar.5 |
| A-539 | Ar.79 | Ar.5 |
| A-540 | Ar.80 | Ar.5 |
| A-541 | CN | Ar.6 |
| A-542 | OCH₃ | Ar.6 |
| A-543 | SCH₃ | Ar.6 |
| A-544 | OCF₃ | Ar.6 |
| A-545 | OCHF₂ | Ar.6 |
| A-546 | methyl | Ar.6 |
| A-547 | iso-propyl | Ar.6 |
| A-548 | ethenyl | Ar.6 |
| A-549 | ethynyl | Ar.6 |
| A-550 | Ar.0 | Ar.6 |
| A-551 | Ar.1 | Ar.6 |
| A-552 | Ar.2 | Ar.6 |
| A-553 | Ar.3 | Ar.6 |
| A-554 | Ar.4 | Ar.6 |
| A-555 | Ar.5 | Ar.6 |
| A-556 | Ar.6 | Ar.6 |
| A-557 | Ar.7 | Ar.6 |
| A-558 | Ar.8 | Ar.6 |
| A-559 | Ar.9 | Ar.6 |
| A-560 | Ar.10 | Ar.6 |
| A-561 | Ar.11 | Ar.6 |
| A-562 | Ar.12 | Ar.6 |
| A-563 | Ar.13 | Ar.6 |
| A-564 | Ar.14 | Ar.6 |
| A-565 | Ar.15 | Ar.6 |
| A-566 | Ar.16 | Ar.6 |
| A-567 | Ar.17 | Ar.6 |
| A-568 | Ar.18 | Ar.6 |
| A-569 | Ar.19 | Ar.6 |
| A-570 | Ar.20 | Ar.6 |
| A-571 | Ar.21 | Ar.6 |
| A-572 | Ar.22 | Ar.6 |
| A-573 | Ar.23 | Ar.6 |
| A-574 | Ar.24 | Ar.6 |
| A-575 | Ar.25 | Ar.6 |
| A-576 | Ar.26 | Ar.6 |
| A-577 | Ar.27 | Ar.6 |
| A-578 | Ar.28 | Ar.6 |
| A-579 | Ar.29 | Ar.6 |
| A-580 | Ar.30 | Ar.6 |
| A-581 | Ar.31 | Ar.6 |
| A-582 | Ar.32 | Ar.6 |
| A-583 | Ar.33 | Ar.6 |
| A-584 | Ar.34 | Ar.6 |
| A-585 | Ar.35 | Ar.6 |
| A-586 | Ar.36 | Ar.6 |
| A-587 | Ar.37 | Ar.6 |
| A-588 | Ar.38 | Ar.6 |
| A-589 | Ar.39 | Ar.6 |
| A-590 | Ar.40 | Ar.6 |
| A-591 | Ar.41 | Ar.6 |
| A-592 | Ar.42 | Ar.6 |
| A-593 | Ar.43 | Ar.6 |
| A-594 | Ar.44 | Ar.6 |
| A-595 | Ar.45 | Ar.6 |
| A-596 | Ar.46 | Ar.6 |
| A-597 | Ar.47 | Ar.6 |
| A-598 | Ar.48 | Ar.6 |
| A-599 | Ar.49 | Ar.6 |
| A-600 | Ar.50 | Ar.6 |
| A-601 | Ar.51 | Ar.6 |
| A-602 | Ar.52 | Ar.6 |
| A-603 | Ar.53 | Ar.6 |
| A-604 | Ar.54 | Ar.6 |
| A-605 | Ar.55 | Ar.6 |
| A-606 | Ar.56 | Ar.6 |
| A-607 | Ar.57 | Ar.6 |
| A-608 | Ar.58 | Ar.6 |
| A-609 | Ar.59 | Ar.6 |
| A-610 | Ar.60 | Ar.6 |
| A-611 | Ar.61 | Ar.6 |
| A-612 | Ar.62 | Ar.6 |
| A-613 | Ar.63 | Ar.6 |
| A-614 | Ar.64 | Ar.6 |
| A-615 | Ar.65 | Ar.6 |
| A-616 | Ar.66 | Ar.6 |
| A-617 | Ar.67 | Ar.6 |
| A-618 | Ar.68 | Ar.6 |
| A-619 | Ar.69 | Ar.6 |
| A-620 | Ar.70 | Ar.6 |
| A-621 | Ar.71 | Ar.6 |
| A-622 | Ar.72 | Ar.6 |
| A-623 | Ar.73 | Ar.6 |
| A-624 | Ar.74 | Ar.6 |
| A-625 | Ar.75 | Ar.6 |
| A-626 | Ar.76 | Ar.6 |
| A-627 | Ar.77 | Ar.6 |
| A-628 | Ar.78 | Ar.6 |
| A-629 | Ar.79 | Ar.6 |
| A-630 | Ar.80 | Ar.6 |
| A-631 | CN | Ar.7 |
| A-632 | OCH₃ | Ar.7 |
| A-633 | SCH₃ | Ar.7 |
| A-634 | OCF₃ | Ar.7 |
| A-635 | OCHF₂ | Ar.7 |
| A-636 | methyl | Ar.7 |
| A-637 | iso-propyl | Ar.7 |
| A-638 | ethenyl | Ar.7 |
| A-639 | ethynyl | Ar.7 |
| A-640 | Ar.0 | Ar.7 |
| A-641 | Ar.1 | Ar.7 |
| A-642 | Ar.2 | Ar.7 |
| A-643 | Ar.3 | Ar.7 |
| A-644 | Ar.4 | Ar.7 |
| A-645 | Ar.5 | Ar.7 |
| A-646 | Ar.6 | Ar.7 |
| A-647 | Ar.7 | Ar.7 |
| A-648 | Ar.8 | Ar.7 |
| A-649 | Ar.9 | Ar.7 |
| A-650 | Ar.10 | Ar.7 |
| A-651 | Ar.11 | Ar.7 |
| A-652 | Ar.12 | Ar.7 |
| A-653 | Ar.13 | Ar.7 |
| A-654 | Ar.14 | Ar.7 |
| A-655 | Ar.15 | Ar.7 |
| A-656 | Ar.16 | Ar.7 |
| A-657 | Ar.17 | Ar.7 |
| A-658 | Ar.18 | Ar.7 |
| A-659 | Ar.19 | Ar.7 |
| A-660 | Ar.20 | Ar.7 |
| A-661 | Ar.21 | Ar.7 |
| A-662 | Ar.22 | Ar.7 |
| A-663 | Ar.23 | Ar.7 |
| A-664 | Ar.24 | Ar.7 |
| A-665 | Ar.25 | Ar.7 |
| A-666 | Ar.26 | Ar.7 |
| A-667 | Ar.27 | Ar.7 |
| A-668 | Ar.28 | Ar.7 |
| A-669 | Ar.29 | Ar.7 |
| A-670 | Ar.30 | Ar.7 |
| A-671 | Ar.31 | Ar.7 |
| A-672 | Ar.32 | Ar.7 |
| A-673 | Ar.33 | Ar.7 |
| A-674 | Ar.34 | Ar.7 |
| A-675 | Ar.35 | Ar.7 |
| A-676 | Ar.36 | Ar.7 |
| A-677 | Ar.37 | Ar.7 |
| A-678 | Ar.38 | Ar.7 |
| A-679 | Ar.39 | Ar.7 |
| A-680 | Ar.40 | Ar.7 |
| A-681 | Ar.41 | Ar.7 |
| A-682 | Ar.42 | Ar.7 |
| A-683 | Ar.43 | Ar.7 |
| A-684 | Ar.44 | Ar.7 |
| A-685 | Ar.45 | Ar.7 |
| A-686 | Ar.46 | Ar.7 |
| A-687 | Ar.47 | Ar.7 |
| A-688 | Ar.48 | Ar.7 |
| A-689 | Ar.49 | Ar.7 |
| A-690 | Ar.50 | Ar.7 |
| A-691 | Ar.51 | Ar.7 |
| A-692 | Ar.52 | Ar.7 |
| A-693 | Ar.53 | Ar.7 |
| A-694 | Ar.54 | Ar.7 |
| A-695 | Ar.55 | Ar.7 |
| A-696 | Ar.56 | Ar.7 |
| A-697 | Ar.57 | Ar.7 |
| A-698 | Ar.58 | Ar.7 |
| A-699 | Ar.59 | Ar.7 |
| A-700 | Ar.60 | Ar.7 |
| A-701 | Ar.61 | Ar.7 |
| A-702 | Ar.62 | Ar.7 |
| A-703 | Ar.63 | Ar.7 |
| A-704 | Ar.64 | Ar.7 |
| A-705 | Ar.65 | Ar.7 |
| A-706 | Ar.66 | Ar.7 |
| A-707 | Ar.67 | Ar.7 |
| A-708 | Ar.68 | Ar.7 |
| A-709 | Ar.69 | Ar.7 |
| A-710 | Ar.70 | Ar.7 |
| A-711 | Ar.71 | Ar.7 |
| A-712 | Ar.72 | Ar.7 |
| A-713 | Ar.73 | Ar.7 |
| A-714 | Ar.74 | Ar.7 |
| A-715 | Ar.75 | Ar.7 |
| A-716 | Ar.76 | Ar.7 |
| A-717 | Ar.77 | Ar.7 |
| A-718 | Ar.78 | Ar.7 |
| A-719 | Ar.79 | Ar.7 |
| A-720 | Ar.80 | Ar.7 |
| A-721 | CN | Ar.8 |
| A-722 | OCH₃ | Ar.8 |
| A-723 | SCH₃ | Ar.8 |
| A-724 | OCF₃ | Ar.8 |
| A-725 | OCHF₂ | Ar.8 |
| A-726 | methyl | Ar.8 |
| A-727 | iso-propyl | Ar.8 |
| A-728 | ethenyl | Ar.8 |
| A-729 | ethynyl | Ar.8 |
| A-730 | Ar.0 | Ar.8 |
| A-731 | Ar.1 | Ar.8 |
| A-732 | Ar.2 | Ar.8 |
| A-733 | Ar.3 | Ar.8 |
| A-734 | Ar.4 | Ar.8 |
| A-735 | Ar.5 | Ar.8 |
| A-736 | Ar.6 | Ar.8 |
| A-737 | Ar.7 | Ar.8 |
| A-738 | Ar.8 | Ar.8 |
| A-739 | Ar.9 | Ar.8 |
| A-740 | Ar.10 | Ar.8 |
| A-741 | Ar.11 | Ar.8 |
| A-742 | Ar.12 | Ar.8 |
| A-743 | Ar.13 | Ar.8 |
| A-744 | Ar.14 | Ar.8 |
| A-745 | Ar.15 | Ar.8 |
| A-746 | Ar.16 | Ar.8 |
| A-747 | Ar.17 | Ar.8 |
| A-748 | Ar.18 | Ar.8 |
| A-749 | Ar.19 | Ar.8 |
| A-750 | Ar.20 | Ar.8 |
| A-751 | Ar.21 | Ar.8 |
| A-752 | Ar.22 | Ar.8 |
| A-753 | Ar.23 | Ar.8 |
| A-754 | Ar.24 | Ar.8 |
| A-755 | Ar.25 | Ar.8 |
| A-756 | Ar.26 | Ar.8 |
| A-757 | Ar.27 | Ar.8 |
| A-758 | Ar.28 | Ar.8 |
| A-759 | Ar.29 | Ar.8 |
| A-760 | Ar.30 | Ar.8 |
| A-761 | Ar.31 | Ar.8 |
| A-762 | Ar.32 | Ar.8 |
| A-763 | Ar.33 | Ar.8 |
| A-764 | Ar.34 | Ar.8 |
| A-765 | Ar.35 | Ar.8 |
| A-766 | Ar.36 | Ar.8 |
| A-767 | Ar.37 | Ar.8 |
| A-768 | Ar.38 | Ar.8 |
| A-769 | Ar.39 | Ar.8 |
| A-770 | Ar.40 | Ar.8 |
| A-771 | Ar.41 | Ar.8 |
| A-772 | Ar.42 | Ar.8 |
| A-773 | Ar.43 | Ar.8 |
| A-774 | Ar.44 | Ar.8 |
| A-775 | Ar.45 | Ar.8 |
| A-776 | Ar.46 | Ar.8 |
| A-777 | Ar.47 | Ar.8 |
| A-778 | Ar.48 | Ar.8 |
| A-779 | Ar.49 | Ar.8 |
| A-780 | Ar.50 | Ar.8 |
| A-781 | Ar.51 | Ar.8 |
| A-782 | Ar.52 | Ar.8 |
| A-783 | Ar.53 | Ar.8 |
| A-784 | Ar.54 | Ar.8 |
| A-785 | Ar.55 | Ar.8 |
| A-786 | Ar.56 | Ar.8 |
| A-787 | Ar.57 | Ar.8 |
| A-788 | Ar.58 | Ar.8 |
| A-789 | Ar.59 | Ar.8 |
| A-790 | Ar.60 | Ar.8 |
| A-791 | Ar.61 | Ar.8 |
| A-792 | Ar.62 | Ar.8 |
| A-793 | Ar.63 | Ar.8 |
| A-794 | Ar.64 | Ar.8 |
| A-795 | Ar.65 | Ar.8 |
| A-796 | Ar.66 | Ar.8 |
| A-797 | Ar.67 | Ar.8 |
| A-798 | Ar.68 | Ar.8 |
| A-799 | Ar.69 | Ar.8 |
| A-800 | Ar.70 | Ar.8 |
| A-801 | Ar.71 | Ar.8 |
| A-802 | Ar.72 | Ar.8 |
| A-803 | Ar.73 | Ar.8 |
| A-804 | Ar.74 | Ar.8 |
| A-805 | Ar.75 | Ar.8 |
| A-806 | Ar.76 | Ar.8 |
| A-807 | Ar.77 | Ar.8 |
| A-808 | Ar.78 | Ar.8 |
| A-809 | Ar.79 | Ar.8 |
| A-810 | Ar.80 | Ar.8 |
| A-811 | CN | Ar.9 |
| A-812 | OCH₃ | Ar.9 |
| A-813 | SCH₃ | Ar.9 |
| A-814 | OCF₃ | Ar.9 |
| A-815 | OCHF₂ | Ar.9 |
| A-816 | methyl | Ar.9 |
| A-817 | iso-propyl | Ar.9 |
| A-818 | ethenyl | Ar.9 |
| A-819 | ethynyl | Ar.9 |
| A-820 | Ar.0 | Ar.9 |
| A-821 | Ar.1 | Ar.9 |
| A-822 | Ar.2 | Ar.9 |
| A-823 | Ar.3 | Ar.9 |
| A-824 | Ar.4 | Ar.9 |
| A-825 | Ar.5 | Ar.9 |
| A-826 | Ar.6 | Ar.9 |
| A-827 | Ar.7 | Ar.9 |
| A-828 | Ar.8 | Ar.9 |
| A-829 | Ar.9 | Ar.9 |
| A-830 | Ar.10 | Ar.9 |
| A-831 | Ar.11 | Ar.9 |
| A-832 | Ar.12 | Ar.9 |
| A-833 | Ar.13 | Ar.9 |
| A-834 | Ar.14 | Ar.9 |
| A-835 | Ar.15 | Ar.9 |
| A-836 | Ar.16 | Ar.9 |
| A-837 | Ar.17 | Ar.9 |
| A-838 | Ar.18 | Ar.9 |
| A-839 | Ar.19 | Ar.9 |
| A-840 | Ar.20 | Ar.9 |
| A-841 | Ar.21 | Ar.9 |
| A-842 | Ar.22 | Ar.9 |
| A-843 | Ar.23 | Ar.9 |
| A-844 | Ar.24 | Ar.9 |
| A-845 | Ar.25 | Ar.9 |
| A-846 | Ar.26 | Ar.9 |
| A-847 | Ar.27 | Ar.9 |
| A-848 | Ar.28 | Ar.9 |
| A-849 | Ar.29 | Ar.9 |
| A-850 | Ar.30 | Ar.9 |
| A-851 | Ar.31 | Ar.9 |
| A-852 | Ar.32 | Ar.9 |
| A-853 | Ar.33 | Ar.9 |
| A-854 | Ar.34 | Ar.9 |
| A-855 | Ar.35 | Ar.9 |
| A-856 | Ar.36 | Ar.9 |
| A-857 | Ar.37 | Ar.9 |
| A-858 | Ar.38 | Ar.9 |
| A-859 | Ar.39 | Ar.9 |
| A-860 | Ar.40 | Ar.9 |
| A-861 | Ar.41 | Ar.9 |
| A-862 | Ar.42 | Ar.9 |
| A-863 | Ar.43 | Ar.9 |
| A-864 | Ar.44 | Ar.9 |
| A-865 | Ar.45 | Ar.9 |
| A-866 | Ar.46 | Ar.9 |
| A-867 | Ar.47 | Ar.9 |
| A-868 | Ar.48 | Ar.9 |
| A-869 | Ar.49 | Ar.9 |
| A-870 | Ar.50 | Ar.9 |
| A-871 | Ar.51 | Ar.9 |
| A-872 | Ar.52 | Ar.9 |
| A-873 | Ar.53 | Ar.9 |
| A-874 | Ar.54 | Ar.9 |
| A-875 | Ar.55 | Ar.9 |
| A-876 | Ar.56 | Ar.9 |
| A-877 | Ar.57 | Ar.9 |
| A-878 | Ar.58 | Ar.9 |
| A-879 | Ar.59 | Ar.9 |
| A-880 | Ar.60 | Ar.9 |
| A-881 | Ar.61 | Ar.9 |
| A-882 | Ar.62 | Ar.9 |
| A-883 | Ar.63 | Ar.9 |
| A-884 | Ar.64 | Ar.9 |
| A-885 | Ar.65 | Ar.9 |
| A-886 | Ar.66 | Ar.9 |
| A-887 | Ar.67 | Ar.9 |
| A-888 | Ar.68 | Ar.9 |
| A-889 | Ar.69 | Ar.9 |
| A-890 | Ar.70 | Ar.9 |
| A-891 | Ar.71 | Ar.9 |
| A-892 | Ar.72 | Ar.9 |
| A-893 | Ar.73 | Ar.9 |
| A-894 | Ar.74 | Ar.9 |
| A-895 | Ar.75 | Ar.9 |
| A-896 | Ar.76 | Ar.9 |
| A-897 | Ar.77 | Ar.9 |
| A-898 | Ar.78 | Ar.9 |
| A-899 | Ar.79 | Ar.9 |
| A-900 | Ar.80 | Ar.9 |
| A-901 | CN | Ar.10 |
| A-902 | OCH₃ | Ar.10 |
| A-903 | SCH₃ | Ar.10 |
| A-904 | OCF₃ | Ar.10 |
| A-905 | OCHF₂ | Ar.10 |
| A-906 | methyl | Ar.10 |
| A-907 | iso-propyl | Ar.10 |
| A-908 | ethenyl | Ar.10 |
| A-909 | ethynyl | Ar.10 |
| A-910 | Ar.0 | Ar.10 |
| A-911 | Ar.1 | Ar.10 |
| A-912 | Ar.2 | Ar.10 |
| A-913 | Ar.3 | Ar.10 |
| A-914 | Ar.4 | Ar.10 |
| A-915 | Ar.5 | Ar.10 |
| A-916 | Ar.6 | Ar.10 |
| A-917 | Ar.7 | Ar.10 |
| A-918 | Ar.8 | Ar.10 |
| A-919 | Ar.9 | Ar.10 |
| A-920 | Ar.10 | Ar.10 |
| A-921 | Ar.11 | Ar.10 |
| A-922 | Ar.12 | Ar.10 |
| A-923 | Ar.13 | Ar.10 |
| A-924 | Ar.14 | Ar.10 |
| A-925 | Ar.15 | Ar.10 |
| A-926 | Ar.16 | Ar.10 |
| A-927 | Ar.17 | Ar.10 |
| A-928 | Ar.18 | Ar.10 |
| A-929 | Ar.19 | Ar.10 |
| A-930 | Ar.20 | Ar.10 |
| A-931 | Ar.21 | Ar.10 |
| A-932 | Ar.22 | Ar.10 |
| A-933 | Ar.23 | Ar.10 |
| A-934 | Ar.24 | Ar.10 |
| A-935 | Ar.25 | Ar.10 |
| A-936 | Ar.26 | Ar.10 |
| A-937 | Ar.27 | Ar.10 |
| A-938 | Ar.28 | Ar.10 |
| A-939 | Ar.29 | Ar.10 |
| A-940 | Ar.30 | Ar.10 |
| A-941 | Ar.31 | Ar.10 |
| A-942 | Ar.32 | Ar.10 |
| A-943 | Ar.33 | Ar.10 |
| A-944 | Ar.34 | Ar.10 |
| A-945 | Ar.35 | Ar.10 |
| A-946 | Ar.36 | Ar.10 |
| A-947 | Ar.37 | Ar.10 |
| A-948 | Ar.38 | Ar.10 |
| A-949 | Ar.39 | Ar.10 |
| A-950 | Ar.40 | Ar.10 |
| A-951 | Ar.41 | Ar.10 |
| A-952 | Ar.42 | Ar.10 |
| A-953 | Ar.43 | Ar.10 |
| A-954 | Ar.44 | Ar.10 |
| A-955 | Ar.45 | Ar.10 |
| A-956 | Ar.46 | Ar.10 |
| A-957 | Ar.47 | Ar.10 |
| A-958 | Ar.48 | Ar.10 |
| A-959 | Ar.49 | Ar.10 |
| A-960 | Ar.50 | Ar.10 |
| A-961 | Ar.51 | Ar.10 |
| A-962 | Ar.52 | Ar.10 |
| A-963 | Ar.53 | Ar.10 |
| A-964 | Ar.54 | Ar.10 |
| A-965 | Ar.55 | Ar.10 |
| A-966 | Ar.56 | Ar.10 |
| A-967 | Ar.57 | Ar.10 |
| A-968 | Ar.58 | Ar.10 |
| A-969 | Ar.59 | Ar.10 |
| A-970 | Ar.60 | Ar.10 |
| A-971 | Ar.61 | Ar.10 |
| A-972 | Ar.62 | Ar.10 |
| A-973 | Ar.63 | Ar.10 |
| A-974 | Ar.64 | Ar.10 |
| A-975 | Ar.65 | Ar.10 |
| A-976 | Ar.66 | Ar.10 |
| A-977 | Ar.67 | Ar.10 |
| A-978 | Ar.68 | Ar.10 |
| A-979 | Ar.69 | Ar.10 |
| A-980 | Ar.70 | Ar.10 |
| A-981 | Ar.71 | Ar.10 |
| A-982 | Ar.72 | Ar.10 |
| A-983 | Ar.73 | Ar.10 |
| A-984 | Ar.74 | Ar.10 |
| A-985 | Ar.75 | Ar.10 |
| A-986 | Ar.76 | Ar.10 |
| A-987 | Ar.77 | Ar.10 |
| A-988 | Ar.78 | Ar.10 |
| A-989 | Ar.79 | Ar.10 |
| A-990 | Ar.80 | Ar.10 |
| A-991 | CN | Ar.11 |
| A-992 | OCH₃ | Ar.11 |
| A-993 | SCH₃ | Ar.11 |
| A-994 | OCF₃ | Ar.11 |
| A-995 | OCHF₂ | Ar.11 |
| A-996 | methyl | Ar.11 |
| A-997 | iso-propyl | Ar.11 |
| A-998 | ethenyl | Ar.11 |
| A-999 | ethynyl | Ar.11 |
| A-1000 | Ar.0 | Ar.11 |
| A-1001 | Ar.1 | Ar.11 |
| A-1002 | Ar.2 | Ar.11 |
| A-1003 | Ar.3 | Ar.11 |
| A-1004 | Ar.4 | Ar.11 |
| A-1005 | Ar.5 | Ar.11 |
| A-1006 | Ar.6 | Ar.11 |
| A-1007 | Ar.7 | Ar.11 |
| A-1008 | Ar.8 | Ar.11 |
| A-1009 | Ar.9 | Ar.11 |
| A-1010 | Ar.10 | Ar.11 |
| A-1011 | Ar.11 | Ar.11 |
| A-1012 | Ar.12 | Ar.11 |
| A-1013 | Ar.13 | Ar.11 |
| A-1014 | Ar.14 | Ar.11 |
| A-1015 | Ar.15 | Ar.11 |
| A-1016 | Ar.16 | Ar.11 |
| A-1017 | Ar.17 | Ar.11 |
| A-1018 | Ar.18 | Ar.11 |
| A-1019 | Ar.19 | Ar.11 |
| A-1020 | Ar.20 | Ar.11 |
| A-1021 | Ar.21 | Ar.11 |
| A-1022 | Ar.22 | Ar.11 |
| A-1023 | Ar.23 | Ar.11 |
| A-1024 | Ar.24 | Ar.11 |
| A-1025 | Ar.25 | Ar.11 |
| A-1026 | Ar.26 | Ar.11 |
| A-1027 | Ar.27 | Ar.11 |
| A-1028 | Ar.28 | Ar.11 |
| A-1029 | Ar.29 | Ar.11 |
| A-1030 | Ar.30 | Ar.11 |
| A-1031 | Ar.31 | Ar.11 |
| A-1032 | Ar.32 | Ar.11 |
| A-1033 | Ar.33 | Ar.11 |
| A-1034 | Ar.34 | Ar.11 |
| A-1035 | Ar.35 | Ar.11 |
| A-1036 | Ar.36 | Ar.11 |
| A-1037 | Ar.37 | Ar.11 |
| A-1038 | Ar.38 | Ar.11 |
| A-1039 | Ar.39 | Ar.11 |
| A-1040 | Ar.40 | Ar.11 |
| A-1041 | Ar.41 | Ar.11 |
| A-1042 | Ar.42 | Ar.11 |
| A-1043 | Ar.43 | Ar.11 |
| A-1044 | Ar.44 | Ar.11 |
| A-1045 | Ar.45 | Ar.11 |
| A-1046 | Ar.46 | Ar.11 |
| A-1047 | Ar.47 | Ar.11 |
| A-1048 | Ar.48 | Ar.11 |
| A-1049 | Ar.49 | Ar.11 |
| A-1050 | Ar.50 | Ar.11 |
| A-1051 | Ar.51 | Ar.11 |
| A-1052 | Ar.52 | Ar.11 |
| A-1053 | Ar.53 | Ar.11 |
| A-1054 | Ar.54 | Ar.11 |
| A-1055 | Ar.55 | Ar.11 |
| A-1056 | Ar.56 | Ar.11 |
| A-1057 | Ar.57 | Ar.11 |
| A-1058 | Ar.58 | Ar.11 |
| A-1059 | Ar.59 | Ar.11 |
| A-1060 | Ar.60 | Ar.11 |
| A-1061 | Ar.61 | Ar.11 |
| A-1062 | Ar.62 | Ar.11 |
| A-1063 | Ar.63 | Ar.11 |
| A-1064 | Ar.64 | Ar.11 |
| A-1065 | Ar.65 | Ar.11 |
| A-1066 | Ar.66 | Ar.11 |
| A-1067 | Ar.67 | Ar.11 |
| A-1068 | Ar.68 | Ar.11 |
| A-1069 | Ar.69 | Ar.11 |
| A-1070 | Ar.70 | Ar.11 |
| A-1071 | Ar.71 | Ar.11 |
| A-1072 | Ar.72 | Ar.11 |
| A-1073 | Ar.73 | Ar.11 |
| A-1074 | Ar.74 | Ar.11 |
| A-1075 | Ar.75 | Ar.11 |
| A-1076 | Ar.76 | Ar.11 |
| A-1077 | Ar.77 | Ar.11 |
| A-1078 | Ar.78 | Ar.11 |
| A-1079 | Ar.79 | Ar.11 |
| A-1080 | Ar.80 | Ar.11 |
| A-1081 | CN | Ar.12 |
| A-1082 | OCH₃ | Ar.12 |
| A-1083 | SCH₃ | Ar.12 |
| A-1084 | OCF₃ | Ar.12 |
| A-1085 | OCHF₂ | Ar.12 |
| A-1086 | methyl | Ar.12 |
| A-1087 | iso-propyl | Ar.12 |
| A-1088 | ethenyl | Ar.12 |
| A-1089 | ethynyl | Ar.12 |
| A-1090 | Ar.0 | Ar.12 |
| A-1091 | Ar.1 | Ar.12 |
| A-1092 | Ar.2 | Ar.12 |
| A-1093 | Ar.3 | Ar.12 |
| A-1094 | Ar.4 | Ar.12 |
| A-1095 | Ar.5 | Ar.12 |
| A-1096 | Ar.6 | Ar.12 |
| A-1097 | Ar.7 | Ar.12 |
| A-1098 | Ar.8 | Ar.12 |
| A-1099 | Ar.9 | Ar.12 |
| A-1100 | Ar.10 | Ar.12 |
| A-1101 | Ar.11 | Ar.12 |
| A-1102 | Ar.12 | Ar.12 |
| A-1103 | Ar.13 | Ar.12 |
| A-1104 | Ar.14 | Ar.12 |
| A-1105 | Ar.15 | Ar.12 |
| A-1106 | Ar.16 | Ar.12 |
| A-1107 | Ar.17 | Ar.12 |
| A-1108 | Ar.18 | Ar.12 |
| A-1109 | Ar.19 | Ar.12 |
| A-1110 | Ar.20 | Ar.12 |
| A-1111 | Ar.21 | Ar.12 |
| A-1112 | Ar.22 | Ar.12 |
| A-1113 | Ar.23 | Ar.12 |
| A-1114 | Ar.24 | Ar.12 |
| A-1115 | Ar.25 | Ar.12 |
| A-1116 | Ar.26 | Ar.12 |
| A-1117 | Ar.27 | Ar.12 |
| A-1118 | Ar.28 | Ar.12 |
| A-1119 | Ar.29 | Ar.12 |
| A-1120 | Ar.30 | Ar.12 |
| A-1121 | Ar.31 | Ar.12 |
| A-1122 | Ar.32 | Ar.12 |
| A-1123 | Ar.33 | Ar.12 |
| A-1124 | Ar.34 | Ar.12 |
| A-1125 | Ar.35 | Ar.12 |
| A-1126 | Ar.36 | Ar.12 |
| A-1127 | Ar.37 | Ar.12 |
| A-1128 | Ar.38 | Ar.12 |
| A-1129 | Ar.39 | Ar.12 |
| A-1130 | Ar.40 | Ar.12 |
| A-1131 | Ar.41 | Ar.12 |
| A-1132 | Ar.42 | Ar.12 |
| A-1133 | Ar.43 | Ar.12 |
| A-1134 | Ar.44 | Ar.12 |
| A-1135 | Ar.45 | Ar.12 |
| A-1136 | Ar.46 | Ar.12 |
| A-1137 | Ar.47 | Ar.12 |
| A-1138 | Ar.48 | Ar.12 |
| A-1139 | Ar.49 | Ar.12 |
| A-1140 | Ar.50 | Ar.12 |
| A-1141 | Ar.51 | Ar.12 |
| A-1142 | Ar.52 | Ar.12 |
| A-1143 | Ar.53 | Ar.12 |
| A-1144 | Ar.54 | Ar.12 |
| A-1145 | Ar.55 | Ar.12 |
| A-1146 | Ar.56 | Ar.12 |
| A-1147 | Ar.57 | Ar.12 |
| A-1148 | Ar.58 | Ar.12 |
| A-1149 | Ar.59 | Ar.12 |
| A-1150 | Ar.60 | Ar.12 |
| A-1151 | Ar.61 | Ar.12 |
| A-1152 | Ar.62 | Ar.12 |
| A-1153 | Ar.63 | Ar.12 |
| A-1154 | Ar.64 | Ar.12 |
| A-1155 | Ar.65 | Ar.12 |
| A-1156 | Ar.66 | Ar.12 |
| A-1157 | Ar.67 | Ar.12 |
| A-1158 | Ar.68 | Ar.12 |
| A-1159 | Ar.69 | Ar.12 |
| A-1160 | Ar.70 | Ar.12 |
| A-1161 | Ar.71 | Ar.12 |
| A-1162 | Ar.72 | Ar.12 |
| A-1163 | Ar.73 | Ar.12 |
| A-1164 | Ar.74 | Ar.12 |
| A-1165 | Ar.75 | Ar.12 |
| A-1166 | Ar.76 | Ar.12 |
| A-1167 | Ar.77 | Ar.12 |
| A-1168 | Ar.78 | Ar.12 |
| A-1169 | Ar.79 | Ar.12 |
| A-1170 | Ar.80 | Ar.12 |
| A-1171 | CN | Ar.13 |
| A-1172 | OCH₃ | Ar.13 |
| A-1173 | SCH₃ | Ar.13 |
| A-1174 | OCF₃ | Ar.13 |
| A-1175 | OCHF₂ | Ar.13 |
| A-1176 | methyl | Ar.13 |
| A-1177 | iso-propyl | Ar.13 |
| A-1178 | ethenyl | Ar.13 |
| A-1179 | ethynyl | Ar.13 |
| A-1180 | Ar.0 | Ar.13 |
| A-1181 | Ar.1 | Ar.13 |
| A-1182 | Ar.2 | Ar.13 |
| A-1183 | Ar.3 | Ar.13 |
| A-1184 | Ar.4 | Ar.13 |
| A-1185 | Ar.5 | Ar.13 |
| A-1186 | Ar.6 | Ar.13 |
| A-1187 | Ar.7 | Ar.13 |
| A-1188 | Ar.8 | Ar.13 |
| A-1189 | Ar.9 | Ar.13 |
| A-1190 | Ar.10 | Ar.13 |
| A-1191 | Ar.11 | Ar.3 |
| A-1192 | Ar.12 | Ar.13 |
| A-1193 | Ar.13 | Ar.13 |
| A-1194 | Ar.14 | Ar.13 |
| A-1195 | Ar.15 | Ar.13 |
| A-1196 | Ar.16 | Ar.13 |
| A-1197 | Ar.17 | Ar.13 |
| A-1198 | Ar.18 | Ar.13 |
| A-1199 | Ar.19 | Ar.13 |
| A-1200 | Ar.20 | Ar.13 |
| A-1201 | Ar.21 | Ar.13 |
| A-1202 | Ar.22 | Ar.13 |
| A-1203 | Ar.23 | Ar.13 |
| A-1204 | Ar.24 | Ar.13 |
| A-1205 | Ar.25 | Ar.13 |
| A-1206 | Ar.26 | Ar.13 |
| A-1207 | Ar.27 | Ar.13 |
| A-1208 | Ar.28 | Ar.13 |
| A-1209 | Ar.29 | Ar.13 |
| A-1210 | Ar.30 | Ar.13 |
| A-1211 | Ar.31 | Ar.13 |
| A-1212 | Ar.32 | Ar.13 |
| A-1213 | Ar.33 | Ar.13 |
| A-1214 | Ar.34 | Ar.13 |
| A-1215 | Ar.35 | Ar.13 |
| A-1216 | Ar.36 | Ar.13 |
| A-1217 | Ar.37 | Ar.13 |
| A-1218 | Ar.38 | Ar.13 |
| A-1219 | Ar.39 | Ar.13 |
| A-1220 | Ar.40 | Ar.13 |
| A-1221 | Ar.41 | Ar.13 |
| A-1222 | Ar.42 | Ar.13 |
| A-1223 | Ar.43 | Ar.13 |
| A-1224 | Ar.44 | Ar.13 |
| A-1225 | Ar.45 | Ar.13 |
| A-1226 | Ar.46 | Ar.13 |
| A-1227 | Ar.47 | Ar.13 |
| A-1228 | Ar.48 | Ar.13 |
| A-1229 | Ar.49 | Ar.13 |
| A-1230 | Ar.50 | Ar.13 |
| A-1231 | Ar.51 | Ar.13 |
| A-1232 | Ar.52 | Ar.13 |
| A-1233 | Ar.53 | Ar.13 |
| A-1234 | Ar.54 | Ar.13 |
| A-1235 | Ar.55 | Ar.13 |
| A-1236 | Ar.56 | Ar.13 |
| A-1237 | Ar.57 | Ar.13 |
| A-1238 | Ar.58 | Ar.13 |
| A-1239 | Ar.59 | Ar.13 |
| A-1240 | Ar.60 | Ar.13 |
| A-1241 | Ar.61 | Ar.13 |
| A-1242 | Ar.62 | Ar.13 |
| A-1243 | Ar.63 | Ar.13 |
| A-1244 | Ar.64 | Ar.13 |
| A-1245 | Ar.65 | Ar.13 |
| A-1246 | Ar.66 | Ar.13 |
| A-1247 | Ar.67 | Ar.13 |
| A-1248 | Ar.68 | Ar.13 |
| A-1249 | Ar.69 | Ar.13 |
| A-1250 | Ar.70 | Ar.13 |
| A-1251 | Ar.71 | Ar.13 |
| A-1252 | Ar.72 | Ar.13 |
| A-1253 | Ar.73 | Ar.13 |
| A-1254 | Ar.74 | Ar.13 |
| A-1255 | Ar.75 | Ar.13 |
| A-1256 | Ar.76 | Ar.13 |
| A-1257 | Ar.77 | Ar.13 |
| A-1258 | Ar.78 | Ar.13 |
| A-1259 | Ar.79 | Ar.13 |
| A-1260 | Ar.80 | Ar.13 |
| A-1261 | CN | Ar.14 |
| A-1262 | OCH₃ | Ar.14 |
| A-1263 | SCH₃ | Ar.14 |
| A-1264 | OCF₃ | Ar.14 |
| A-1265 | OCHF₂ | Ar.14 |
| A-1266 | methyl | Ar.14 |
| A-1267 | iso-propyl | Ar.14 |
| A-1268 | ethenyl | Ar.14 |
| A-1269 | ethynyl | Ar.14 |
| A-1270 | Ar.0 | Ar.14 |
| A-1271 | Ar.1 | Ar.14 |
| A-1272 | Ar.2 | Ar.14 |
| A-1273 | Ar.3 | Ar.14 |
| A-1274 | Ar.4 | Ar.14 |
| A-1275 | Ar.5 | Ar.14 |
| A-1276 | Ar.6 | Ar.14 |
| A-1277 | Ar.7 | Ar.14 |
| A-1278 | Ar.8 | Ar.14 |
| A-1279 | Ar.9 | Ar.14 |
| A-1280 | Ar.10 | Ar.14 |
| A-1281 | Ar.11 | Ar.14 |
| A-1282 | Ar.12 | Ar.14 |
| A-1283 | Ar.13 | Ar.14 |
| A-1284 | Ar.14 | Ar.14 |
| A-1285 | Ar.15 | Ar.14 |
| A-1286 | Ar.16 | Ar.14 |
| A-1287 | Ar.17 | Ar.14 |
| A-1288 | Ar.18 | Ar.14 |
| A-1289 | Ar.19 | Ar.14 |
| A-1290 | Ar.20 | Ar.14 |
| A-1291 | Ar.21 | Ar.14 |
| A-1292 | Ar.22 | Ar.14 |
| A-1293 | Ar.23 | Ar.14 |
| A-1294 | Ar.24 | Ar.14 |
| A-1295 | Ar.25 | Ar.14 |
| A-1296 | Ar.26 | Ar.14 |
| A-1297 | Ar.27 | Ar.14 |
| A-1298 | Ar.28 | Ar.14 |
| A-1299 | Ar.29 | Ar.14 |
| A-1300 | Ar.30 | Ar.14 |
| A-1301 | Ar.31 | Ar.14 |
| A-1302 | Ar.32 | Ar.14 |
| A-1303 | Ar.33 | Ar.14 |
| A-1304 | Ar.34 | Ar.14 |
| A-1305 | Ar.35 | Ar.14 |
| A-1306 | Ar.36 | Ar.14 |
| A-1307 | Ar.37 | Ar.14 |
| A-1308 | Ar.38 | Ar.14 |
| A-1309 | Ar.39 | Ar.14 |
| A-1310 | Ar.40 | Ar.14 |
| A-1311 | Ar.41 | Ar.14 |
| A-1312 | Ar.42 | Ar.14 |
| A-1313 | Ar.43 | Ar.14 |
| A-1314 | Ar.44 | Ar.14 |
| A-1315 | Ar.45 | Ar.14 |
| A-1316 | Ar.46 | Ar.14 |
| A-1317 | Ar.47 | Ar.14 |
| A-1318 | Ar.48 | Ar.14 |
| A-1319 | Ar.49 | Ar.14 |
| A-1320 | Ar.50 | Ar.14 |
| A-1321 | Ar.51 | Ar.14 |
| A-1322 | Ar.52 | Ar.14 |
| A-1323 | Ar.53 | Ar.14 |
| A-1324 | Ar.54 | Ar.14 |
| A-1325 | Ar.55 | Ar.14 |
| A-1326 | Ar.56 | Ar.14 |
| A-1327 | Ar.57 | Ar.14 |
| A-1328 | Ar.58 | Ar.14 |
| A-1329 | Ar.59 | Ar.14 |
| A-1330 | Ar.60 | Ar.14 |
| A-1331 | Ar.61 | Ar.14 |
| A-1332 | Ar.62 | Ar.14 |
| A-1333 | Ar.63 | Ar.14 |
| A-1334 | Ar.64 | Ar.14 |
| A-1335 | Ar.65 | Ar.14 |
| A-1336 | Ar.66 | Ar.14 |
| A-1337 | Ar.67 | Ar.14 |
| A-1338 | Ar.68 | Ar.14 |
| A-1339 | Ar.69 | Ar.14 |
| A-1340 | Ar.70 | Ar.14 |
| A-1341 | Ar.71 | Ar.14 |
| A-1342 | Ar.72 | Ar.14 |
| A-1343 | Ar.73 | Ar.14 |
| A-1344 | Ar.74 | Ar.14 |
| A-1345 | Ar.75 | Ar.14 |
| A-1346 | Ar.76 | Ar.14 |
| A-1347 | Ar.77 | Ar.14 |
| A-1348 | Ar.78 | Ar.14 |
| A-1349 | Ar.79 | Ar.14 |
| A-1350 | Ar.80 | Ar.14 |
| A-1351 | CN | Ar.15 |
| A-1352 | OCH₃ | Ar.15 |
| A-1353 | SCH₃ | Ar.15 |
| A-1354 | OCF₃ | Ar.15 |
| A-1355 | OCHF₂ | Ar.15 |
| A-1356 | methyl | Ar.15 |
| A-1357 | iso-propyl | Ar.15 |
| A-1358 | ethenyl | Ar.15 |
| A-1359 | ethynyl | Ar.15 |
| A-1360 | Ar.0 | Ar.15 |
| A-1361 | Ar.1 | Ar.15 |
| A-1362 | Ar.2 | Ar.15 |
| A-1363 | Ar.3 | Ar.15 |
| A-1364 | Ar.4 | Ar.15 |
| A-1365 | Ar.5 | Ar.15 |
| A-1366 | Ar.6 | Ar.15 |
| A-1367 | Ar.7 | Ar.15 |
| A-1368 | Ar.8 | Ar.15 |
| A-1369 | Ar.9 | Ar.15 |
| A-1370 | Ar.10 | Ar.15 |
| A-1371 | Ar.11 | Ar.15 |
| A-1372 | Ar.12 | Ar.15 |
| A-1373 | Ar.13 | Ar.15 |
| A-1374 | Ar.14 | Ar.15 |
| A-1375 | Ar.15 | Ar.15 |
| A-1376 | Ar.16 | Ar.15 |
| A-1377 | Ar.17 | Ar.15 |
| A-1378 | Ar.18 | Ar.15 |
| A-1379 | Ar.19 | Ar.15 |
| A-1380 | Ar.20 | Ar.15 |
| A-1381 | Ar.21 | Ar.15 |
| A-1382 | Ar.22 | Ar.15 |
| A-1383 | Ar.23 | Ar.15 |
| A-1384 | Ar.24 | Ar.15 |
| A-1385 | Ar.25 | Ar.15 |
| A-1386 | Ar.26 | Ar.15 |
| A-1387 | Ar.27 | Ar.15 |
| A-1388 | Ar.28 | Ar.15 |
| A-1389 | Ar.29 | Ar.15 |
| A-1390 | Ar.30 | Ar.15 |
| A-1391 | Ar.31 | Ar.15 |
| A-1392 | Ar.32 | Ar.15 |
| A-1393 | Ar.33 | Ar.15 |
| A-1394 | Ar.34 | Ar.15 |
| A-1395 | Ar.35 | Ar.15 |
| A-1396 | Ar.36 | Ar.15 |
| A-1397 | Ar.37 | Ar.15 |
| A-1398 | Ar.38 | Ar.15 |
| A-1399 | Ar.39 | Ar.15 |
| A-1400 | Ar.40 | Ar.15 |
| A-1401 | Ar.41 | Ar.15 |
| A-1402 | Ar.42 | Ar.15 |
| A-1403 | Ar.43 | Ar.15 |
| A-1404 | Ar.44 | Ar.15 |
| A-1405 | Ar.45 | Ar.15 |
| A-1406 | Ar.46 | Ar.15 |
| A-1407 | Ar.47 | Ar.15 |
| A-1408 | Ar.48 | Ar.15 |
| A-1409 | Ar.49 | Ar.15 |
| A-1410 | Ar.50 | Ar.15 |
| A-1411 | Ar.51 | Ar.15 |
| A-1412 | Ar.52 | Ar.15 |
| A-1413 | Ar.53 | Ar.15 |
| A-1414 | Ar.54 | Ar.15 |
| A-1415 | Ar.55 | Ar.15 |
| A-1416 | Ar.56 | Ar.15 |
| A-1417 | Ar.57 | Ar.15 |
| A-1418 | Ar.58 | Ar.15 |
| A-1419 | Ar.59 | Ar.15 |
| A-1420 | Ar.60 | Ar.15 |
| A-1421 | Ar.61 | Ar.15 |
| A-1422 | Ar.62 | Ar.15 |
| A-1423 | Ar.63 | Ar.15 |
| A-1424 | Ar.64 | Ar.15 |
| A-1425 | Ar.65 | Ar.15 |
| A-1426 | Ar.66 | Ar.15 |
| A-1427 | Ar.67 | Ar.15 |
| A-1428 | Ar.68 | Ar.15 |
| A-1429 | Ar.69 | Ar.15 |
| A-1430 | Ar.70 | Ar.15 |
| A-1431 | Ar.71 | Ar.15 |
| A-1432 | Ar.72 | Ar.15 |
| A-1433 | Ar.73 | Ar.15 |
| A-1434 | Ar.74 | Ar.15 |
| A-1435 | Ar.75 | Ar.15 |
| A-1436 | Ar.76 | Ar.15 |
| A-1437 | Ar.77 | Ar.15 |
| A-1438 | Ar.78 | Ar.15 |
| A-1439 | Ar.79 | Ar.15 |
| A-1440 | Ar.80 | Ar.15 |
| A-1441 | CN | Ar.16 |
| A-1442 | OCH₃ | Ar.16 |
| A-1443 | SCH₃ | Ar.16 |
| A-1444 | OCF₃ | Ar.16 |
| A-1445 | OCHF₂ | Ar.16 |
| A-1446 | methyl | Ar.16 |
| A-1447 | iso-propyl | Ar.16 |
| A-1448 | ethenyl | Ar.16 |
| A-1449 | ethynyl | Ar.16 |
| A-1450 | Ar.0 | Ar.16 |
| A-1451 | Ar.1 | Ar.16 |
| A-1452 | Ar.2 | Ar.16 |
| A-1453 | Ar.3 | Ar.16 |
| A-1454 | Ar.4 | Ar.16 |
| A-1455 | Ar.5 | Ar.16 |
| A-1456 | Ar.6 | Ar.16 |
| A-1457 | Ar.7 | Ar.16 |
| A-1458 | Ar.8 | Ar.16 |
| A-1459 | Ar.9 | Ar.16 |
| A-1460 | Ar.10 | Ar.16 |
| A-1461 | Ar.11 | Ar.16 |
| A-1462 | Ar.12 | Ar.16 |
| A-1463 | Ar.13 | Ar.16 |
| A-1464 | Ar.14 | Ar.16 |
| A-1465 | Ar.15 | Ar.16 |
| A-1466 | CN | Ar.17 |
| A-1467 | OCH₃ | Ar.17 |
| A-1468 | SCH₃ | Ar.17 |
| A-1469 | OCF₃ | Ar.17 |
| A-1470 | OCHF₂ | Ar.17 |
| A-1471 | methyl | Ar.17 |
| A-1472 | iso-propyl | Ar.17 |
| A-1473 | ethenyl | Ar.17 |
| A-1474 | ethynyl | Ar.17 |
| A-1475 | Ar.0 | Ar.17 |
| A-1476 | Ar.1 | Ar.17 |
| A-1477 | Ar.2 | Ar.17 |
| A-1478 | Ar.3 | Ar.17 |
| A-1479 | Ar.4 | Ar.17 |
| A-1480 | Ar.5 | Ar.17 |
| A-1481 | Ar.6 | Ar.17 |
| A-1482 | Ar.7 | Ar.17 |
| A-1483 | Ar.8 | Ar.17 |
| A-1484 | Ar.9 | Ar.17 |
| A-1485 | Ar.10 | Ar.17 |
| A-1486 | Ar.11 | Ar.17 |
| A-1487 | Ar.12 | Ar.17 |
| A-1488 | Ar.13 | Ar.17 |
| A-1489 | Ar.14 | Ar.17 |
| A-1490 | Ar.15 | Ar.17 |
| A-1491 | CN | Ar.18 |
| A-1492 | OCH₃ | Ar.18 |
| A-1493 | SCH₃ | Ar.18 |
| A-1494 | OCF₃ | Ar.18 |
| A-1495 | OCHF₂ | Ar.18 |
| A-1496 | methyl | Ar.18 |
| A-1497 | iso-propyl | Ar.18 |
| A-1498 | ethenyl | Ar.18 |
| A-1499 | ethynyl | Ar.18 |
| A-1500 | Ar.0 | Ar.18 |
| A-1501 | Ar.1 | Ar.18 |
| A-1502 | Ar.2 | Ar.18 |
| A-1503 | Ar.3 | Ar.18 |
| A-1504 | Ar.4 | Ar.18 |
| A-1505 | Ar.5 | Ar.18 |
| A-1506 | Ar.6 | Ar.18 |
| A-1507 | Ar.7 | Ar.18 |
| A-1508 | Ar.8 | Ar.18 |
| A-1509 | Ar.9 | Ar.18 |
| A-1510 | Ar.10 | Ar.18 |
| A-1511 | Ar.11 | Ar.18 |
| A-1512 | Ar.12 | Ar.18 |
| A-1513 | Ar.13 | Ar.18 |
| A-1514 | Ar.14 | Ar.18 |
| A-1515 | Ar.15 | Ar.18 |
| A-1516 | CN | Ar.19 |
| A-1517 | OCH₃ | Ar.19 |
| A-1518 | SCH₃ | Ar.19 |
| A-1519 | OCF₃ | Ar.19 |
| A-1520 | OCHF₂ | Ar.19 |
| A-1521 | methyl | Ar.19 |
| A-1522 | iso-propyl | Ar.19 |
| A-1523 | ethenyl | Ar.19 |
| A-1524 | ethynyl | Ar.19 |
| A-1525 | Ar.0 | Ar.19 |
| A-1526 | Ar.1 | Ar.19 |
| A-1527 | Ar.2 | Ar.19 |
| A-1528 | Ar.3 | Ar.19 |
| A-1529 | Ar.4 | Ar.19 |
| A-1530 | Ar.5 | Ar.19 |
| A-1531 | Ar.6 | Ar.19 |
| A-1532 | Ar.7 | Ar.19 |
| A-1533 | Ar.8 | Ar.19 |
| A-1534 | Ar.9 | Ar.19 |
| A-1535 | Ar.10 | Ar.19 |
| A-1536 | Ar.11 | Ar.19 |
| A-1537 | Ar.12 | Ar.19 |
| A-1538 | Ar.13 | Ar.19 |
| A-1539 | Ar.14 | Ar.19 |
| A-1540 | Ar.15 | Ar.19 |
| A-1541 | CN | Ar.20 |
| A-1542 | OCH₃ | Ar.20 |
| A-1543 | SCH₃ | Ar.20 |
| A-1544 | OCF₃ | Ar.20 |
| A-1545 | OCHF₂ | Ar.20 |
| A-1546 | methyl | Ar.20 |
| A-1547 | iso-propyl | Ar.20 |
| A-1548 | ethenyl | Ar.20 |
| A-1549 | ethynyl | Ar.20 |
| A-1550 | Ar.0 | Ar.20 |
| A-1551 | Ar.1 | Ar.20 |
| A-1552 | Ar.2 | Ar.20 |
| A-1553 | Ar.3 | Ar.20 |
| A-1554 | Ar.4 | Ar.20 |
| A-1555 | Ar.5 | Ar.20 |
| A-1556 | Ar.6 | Ar.20 |
| A-1557 | Ar.7 | Ar.20 |
| A-1558 | Ar.8 | Ar.20 |
| A-1559 | Ar.9 | Ar.20 |
| A-1560 | Ar.10 | Ar.20 |
| A-1561 | Ar.11 | Ar.20 |
| A-1562 | Ar.12 | Ar.20 |
| A-1563 | Ar.13 | Ar.20 |
| A-1564 | Ar.14 | Ar.20 |
| A-1565 | Ar.15 | Ar.20 |
| A-1566 | CN | Ar.21 |
| A-1567 | OCH₃ | Ar.21 |
| A-1568 | SCH₃ | Ar.21 |
| A-1569 | OCF₃ | Ar.21 |
| A-1570 | OCHF₂ | Ar.21 |
| A-1571 | methyl | Ar.21 |
| A-1572 | iso-propyl | Ar.21 |
| A-1573 | ethenyl | Ar.21 |
| A-1574 | ethynyl | Ar.21 |
| A-1575 | Ar.0 | Ar.21 |
| A-1576 | Ar.1 | Ar.21 |
| A-1577 | Ar.2 | Ar.21 |
| A-1578 | Ar.3 | Ar.21 |
| A-1579 | Ar.4 | Ar.21 |
| A-1580 | Ar.5 | Ar.21 |
| A-1581 | Ar.6 | Ar.21 |
| A-1582 | Ar.7 | Ar.21 |
| A-1583 | Ar.8 | Ar.21 |
| A-1584 | Ar.9 | Ar.21 |
| A-1585 | Ar.10 | Ar.21 |
| A-1586 | Ar.11 | Ar.21 |
| A-1587 | Ar.12 | Ar.21 |
| A-1588 | Ar.13 | Ar.21 |
| A-1589 | Ar.14 | Ar.21 |
| A-1590 | Ar.15 | Ar.21 |
| A-1591 | CN | Ar.22 |
| A-1592 | OCH₃ | Ar.22 |
| A-1593 | SCH₃ | Ar.22 |
| A-1594 | OCF₃ | Ar.22 |
| A-1595 | OCHF₂ | Ar.22 |
| A-1596 | methyl | Ar.22 |
| A-1597 | iso-propyl | Ar.22 |
| A-1598 | ethenyl | Ar.22 |
| A-1599 | ethynyl | Ar.22 |
| A-1600 | Ar.0 | Ar.22 |
| A-1601 | Ar.1 | Ar.22 |
| A-1602 | Ar.2 | Ar.22 |
| A-1603 | Ar.3 | Ar.22 |
| A-1604 | Ar.4 | Ar.22 |
| A-1605 | Ar.5 | Ar.22 |
| A-1606 | Ar.6 | Ar.22 |
| A-1607 | Ar.7 | Ar.22 |
| A-1608 | Ar.8 | Ar.22 |
| A-1609 | Ar.9 | Ar.22 |
| A-1610 | Ar.10 | Ar.22 |
| A-1611 | Ar.11 | Ar.22 |
| A-1612 | Ar.12 | Ar.22 |
| A-1613 | Ar.13 | Ar.22 |
| A-1614 | Ar.14 | Ar.22 |
| A-1615 | Ar.15 | Ar.22 |
| A-1616 | CN | Ar.23 |
| A-1617 | OCH₃ | Ar.23 |
| A-1618 | SCH₃ | Ar.23 |
| A-1619 | OCF₃ | Ar.23 |
| A-1620 | OCHF₂ | Ar.23 |
| A-1621 | methyl | Ar.23 |
| A-1622 | iso-propyl | Ar.23 |
| A-1623 | ethenyl | Ar.23 |
| A-1624 | ethynyl | Ar.23 |
| A-1625 | Ar.0 | Ar.23 |
| A-1626 | Ar.1 | Ar.23 |
| A-1627 | Ar.2 | Ar.23 |
| A-1628 | Ar.3 | Ar.23 |
| A-1629 | Ar.4 | Ar.23 |
| A-1630 | Ar.5 | Ar.23 |
| A-1631 | Ar.6 | Ar.23 |
| A-1632 | Ar.7 | Ar.23 |
| A-1633 | Ar.8 | Ar.23 |
| A-1634 | Ar.9 | Ar.23 |
| A-1635 | Ar.10 | Ar.23 |
| A-1636 | Ar.11 | Ar.23 |
| A-1637 | Ar.12 | Ar.23 |
| A-1638 | Ar.13 | Ar.23 |
| A-1639 | Ar.14 | Ar.23 |
| A-1640 | Ar.15 | Ar.23 |
| A-1641 | CN | Ar.24 |
| A-1642 | OCH₃ | Ar.24 |
| A-1643 | SCH₃ | Ar.24 |
| A-1644 | OCF₃ | Ar.24 |
| A-1645 | OCHF₂ | Ar.24 |
| A-1646 | methyl | Ar.24 |
| A-1647 | iso-propyl | Ar.24 |
| A-1648 | ethenyl | Ar.24 |
| A-1649 | ethynyl | Ar.24 |
| A-1650 | Ar.0 | Ar.24 |
| A-1651 | Ar.1 | Ar.24 |
| A-1652 | Ar.2 | Ar.24 |
| A-1653 | Ar.3 | Ar.24 |
| A-1654 | Ar.4 | Ar.24 |
| A-1655 | Ar.5 | Ar.24 |
| A-1656 | Ar.6 | Ar.24 |
| A-1657 | Ar.7 | Ar.24 |
| A-1658 | Ar.8 | Ar.24 |
| A-1659 | Ar.9 | Ar.24 |
| A-1660 | Ar.10 | Ar.24 |
| A-1661 | Ar.11 | Ar.24 |
| A-1662 | Ar.12 | Ar.24 |
| A-1663 | Ar.13 | Ar.24 |
| A-1664 | Ar.14 | Ar.24 |
| A-1665 | Ar.15 | Ar.24 |
| A-1666 | CN | Ar.25 |
| A-1667 | OCH₃ | Ar.25 |
| A-1668 | SCH₃ | Ar.25 |
| A-1669 | OCF₃ | Ar.25 |
| A-1670 | OCHF₂ | Ar.25 |
| A-1671 | methyl | Ar.25 |
| A-1672 | iso-propyl | Ar.25 |
| A-1673 | ethenyl | Ar.25 |
| A-1674 | ethynyl | Ar.25 |
| A-1675 | Ar.0 | Ar.25 |
| A-1676 | Ar.1 | Ar.25 |
| A-1677 | Ar.2 | Ar.25 |
| A-1678 | Ar.3 | Ar.25 |
| A-1679 | Ar.4 | Ar.25 |
| A-1680 | Ar.5 | Ar.25 |
| A-1681 | Ar.6 | Ar.25 |
| A-1682 | Ar.7 | Ar.25 |
| A-1683 | Ar.8 | Ar.25 |
| A-1684 | Ar.9 | Ar.25 |
| A-1685 | Ar.10 | Ar.25 |
| A-1686 | Ar.11 | Ar.25 |
| A-1687 | Ar.12 | Ar.25 |
| A-1688 | Ar.13 | Ar.25 |
| A-1689 | Ar.14 | Ar.25 |
| A-1690 | Ar.15 | Ar.25 |
| A-1691 | CN | Ar.26 |
| A-1692 | OCH₃ | Ar.26 |
| A-1693 | SCH₃ | Ar.26 |
| A-1694 | OCF₃ | Ar.26 |
| A-1695 | OCHF₂ | Ar.26 |
| A-1696 | methyl | Ar.26 |
| A-1697 | iso-propyl | Ar.26 |
| A-1698 | ethenyl | Ar.26 |
| A-1699 | ethynyl | Ar.26 |
| A-1700 | Ar.0 | Ar.26 |
| A-1701 | Ar.1 | Ar.26 |
| A-1702 | Ar.2 | Ar.26 |
| A-1703 | Ar.3 | Ar.26 |
| A-1704 | Ar.4 | Ar.26 |
| A-1705 | Ar.5 | Ar.26 |
| A-1706 | Ar.6 | Ar.26 |
| A-1707 | Ar.7 | Ar.26 |
| A-1708 | Ar.8 | Ar.26 |
| A-1709 | Ar.9 | Ar.26 |
| A-1710 | Ar.10 | Ar.26 |
| A-1711 | Ar.11 | Ar.26 |
| A-1712 | Ar.12 | Ar.26 |
| A-1713 | Ar.13 | Ar.26 |
| A-1714 | Ar.14 | Ar.26 |
| A-1715 | Ar.15 | Ar.26 |
| A-1716 | CN | Ar.27 |
| A-1717 | OCH₃ | Ar.27 |
| A-1718 | SCH₃ | Ar.27 |
| A-1719 | OCF₃ | Ar.27 |
| A-1720 | OCHF₂ | Ar.27 |
| A-1721 | methyl | Ar.27 |
| A-1722 | iso-propyl | Ar.27 |
| A-1723 | ethenyl | Ar.27 |
| A-1724 | ethynyl | Ar.27 |
| A-1725 | Ar.0 | Ar.27 |
| A-1726 | Ar.1 | Ar.27 |
| A-1727 | Ar.2 | Ar.27 |
| A-1728 | Ar.3 | Ar.27 |
| A-1729 | Ar.4 | Ar.27 |
| A-1730 | Ar.5 | Ar.27 |
| A-1731 | Ar.6 | Ar.27 |
| A-1732 | Ar.7 | Ar.27 |
| A-1733 | Ar.8 | Ar.27 |
| A-1734 | Ar.9 | Ar.27 |
| A-1735 | Ar.10 | Ar.27 |
| A-1736 | Ar.11 | Ar.27 |
| A-1737 | Ar.12 | Ar.27 |
| A-1738 | Ar.13 | Ar.27 |
| A-1739 | Ar.14 | Ar.27 |
| A-1740 | Ar.15 | Ar.27 |
| A-1741 | CN | Ar.28 |
| A-1742 | OCH₃ | Ar.28 |
| A-1743 | SCH₃ | Ar.28 |
| A-1744 | OCF₃ | Ar.28 |
| A-1745 | OCHF₂ | Ar.28 |
| A-1746 | methyl | Ar.28 |
| A-1747 | iso-propyl | Ar.28 |
| A-1748 | ethenyl | Ar.28 |
| A-1749 | ethynyl | Ar.28 |
| A-1750 | Ar.0 | Ar.28 |
| A-1751 | Ar.1 | Ar.28 |
| A-1752 | Ar.2 | Ar.28 |
| A-1753 | Ar.3 | Ar.28 |
| A-1754 | Ar.4 | Ar.28 |
| A-1755 | Ar.5 | Ar.28 |
| A-1756 | Ar.6 | Ar.28 |
| A-1757 | Ar.7 | Ar.28 |
| A-1758 | Ar.8 | Ar.28 |
| A-1759 | Ar.9 | Ar.28 |
| A-1760 | Ar.10 | Ar.28 |
| A-1761 | Ar.11 | Ar.28 |
| A-1762 | Ar.12 | Ar.28 |
| A-1763 | Ar.13 | Ar.28 |
| A-1764 | Ar.14 | Ar.28 |
| A-1765 | Ar.15 | Ar.28 |
| A-1766 | CN | Ar.29 |
| A-1767 | OCH₃ | Ar.29 |
| A-1768 | SCH₃ | Ar.29 |
| A-1769 | OCF₃ | Ar.29 |
| A-1770 | OCHF₂ | Ar.29 |
| A-1771 | methyl | Ar.29 |
| A-1772 | iso-propyl | Ar.29 |
| A-1773 | ethenyl | Ar.29 |
| A-1774 | ethynyl | Ar.29 |
| A-1775 | Ar.0 | Ar.29 |
| A-1776 | Ar.1 | Ar.29 |
| A-1777 | Ar.2 | Ar.29 |
| A-1778 | Ar.3 | Ar.29 |
| A-1779 | Ar.4 | Ar.29 |
| A-1780 | Ar.5 | Ar.29 |
| A-1781 | Ar.6 | Ar.29 |
| A-1782 | Ar.7 | Ar.29 |
| A-1783 | Ar.8 | Ar.29 |
| A-1784 | Ar.9 | Ar.29 |
| A-1785 | Ar.10 | Ar.29 |
| A-1786 | Ar.11 | Ar.29 |
| A-1787 | Ar.12 | Ar.29 |
| A-1788 | Ar.13 | Ar.29 |
| A-1789 | Ar.14 | Ar.29 |
| A-1790 | Ar.15 | Ar.29 |
| A-1791 | CN | Ar.30 |
| A-1792 | OCH₃ | Ar.30 |
| A-1793 | SCH₃ | Ar.30 |
| A-1794 | OCF₃ | Ar.30 |
| A-1795 | OCHF₂ | Ar.30 |
| A-1796 | methyl | Ar.30 |
| A-1797 | iso-propyl | Ar.30 |
| A-1798 | ethenyl | Ar.30 |
| A-1799 | ethynyl | Ar.30 |
| A-1800 | Ar.0 | Ar.30 |
| A-1801 | Ar.1 | Ar.30 |
| A-1802 | Ar.2 | Ar.30 |
| A-1803 | Ar.3 | Ar.30 |
| A-1804 | Ar.4 | Ar.30 |
| A-1805 | Ar.5 | Ar.30 |
| A-1806 | Ar.6 | Ar.30 |
| A-1807 | Ar.7 | Ar.30 |
| A-1808 | Ar.8 | Ar.30 |
| A-1809 | Ar.9 | Ar.30 |
| A-1810 | Ar.10 | Ar.30 |
| A-1811 | Ar.11 | Ar.30 |
| A-1812 | Ar.12 | Ar.30 |
| A-1813 | Ar.13 | Ar.30 |
| A-1814 | Ar.14 | Ar.30 |
| A-1815 | Ar.15 | Ar.30 |
| A-1816 | CN | Ar.31 |
| A-1817 | OCH₃ | Ar.31 |
| A-1818 | SCH₃ | Ar.31 |
| A-1819 | OCF₃ | Ar.31 |
| A-1820 | OCHF₂ | Ar.31 |
| A-1821 | methyl | Ar.31 |
| A-1822 | iso-propyl | Ar.31 |
| A-1823 | ethenyl | Ar.31 |
| A-1824 | ethynyl | Ar.31 |
| A-1825 | Ar.0 | Ar.31 |
| A-1826 | Ar.1 | Ar.31 |
| A-1827 | Ar.2 | Ar.31 |
| A-1828 | Ar.3 | Ar.31 |
| A-1829 | Ar.4 | Ar.31 |
| A-1830 | Ar.5 | Ar.31 |
| A-1831 | Ar.6 | Ar.31 |
| A-1832 | Ar.7 | Ar.31 |
| A-1833 | Ar.8 | Ar.31 |
| A-1834 | Ar.9 | Ar.31 |
| A-1835 | Ar.10 | Ar.31 |
| A-1836 | Ar.11 | Ar.31 |
| A-1837 | Ar.12 | Ar.31 |
| A-1838 | Ar.13 | Ar.31 |
| A-1839 | Ar.14 | Ar.31 |
| A-1840 | Ar.15 | Ar.31 |
| A-1841 | CN | Ar.32 |
| A-1842 | OCH₃ | Ar.32 |
| A-1843 | SCH₃ | Ar.32 |
| A-1844 | OCF₃ | Ar.32 |
| A-1845 | OCHF₂ | Ar.32 |
| A-1846 | methyl | Ar.32 |
| A-1847 | iso-propyl | Ar.32 |
| A-1848 | ethenyl | Ar.32 |
| A-1849 | ethynyl | Ar.32 |
| A-1850 | Ar.0 | Ar.32 |
| A-1851 | Ar.1 | Ar.32 |
| A-1852 | Ar.2 | Ar.32 |
| A-1853 | Ar.3 | Ar.32 |
| A-1854 | Ar.4 | Ar.32 |
| A-1855 | Ar.5 | Ar.32 |
| A-1856 | Ar.6 | Ar.32 |
| A-1857 | Ar.7 | Ar.32 |
| A-1858 | Ar.8 | Ar.32 |
| A-1859 | Ar.9 | Ar.32 |
| A-1860 | Ar.10 | Ar.32 |
| A-1861 | Ar.11 | Ar.32 |
| A-1862 | Ar.12 | Ar.32 |
| A-1863 | Ar.13 | Ar.32 |
| A-1864 | Ar.14 | Ar.32 |
| A-1865 | Ar.15 | Ar.32 |
| A-1866 | CN | Ar.33 |
| A-1867 | OCH₃ | Ar.33 |
| A-1868 | SCH₃ | Ar.33 |
| A-1869 | OCF₃ | Ar.33 |
| A-1870 | OCHF₂ | Ar.33 |
| A-1871 | methyl | Ar.33 |
| A-1872 | iso-propyl | Ar.33 |
| A-1873 | ethenyl | Ar.33 |
| A-1874 | ethynyl | Ar.33 |
| A-1875 | Ar.0 | Ar.33 |
| A-1876 | Ar.1 | Ar.33 |
| A-1877 | Ar.2 | Ar.33 |
| A-1878 | Ar.3 | Ar.33 |
| A-1879 | Ar.4 | Ar.33 |
| A-1880 | Ar.5 | Ar.33 |
| A-1881 | Ar.6 | Ar.33 |
| A-1882 | Ar.7 | Ar.33 |
| A-1883 | Ar.8 | Ar.33 |
| A-1884 | Ar.9 | Ar.33 |
| A-1885 | Ar.10 | Ar.33 |
| A-1886 | Ar.11 | Ar.33 |
| A-1887 | Ar.12 | Ar.33 |
| A-1888 | Ar.13 | Ar.33 |
| A-1889 | Ar.14 | Ar.33 |
| A-1890 | Ar.15 | Ar.33 |
| A-1891 | CN | Ar.34 |
| A-1892 | OCH₃ | Ar.34 |
| A-1893 | SCH₃ | Ar.34 |
| A-1894 | OCF₃ | Ar.34 |
| A-1895 | OCHF₂ | Ar.34 |
| A-1896 | methyl | Ar.34 |
| A-1897 | iso-propyl | Ar.34 |
| A-1898 | ethenyl | Ar.34 |
| A-1899 | ethynyl | Ar.34 |
| A-1900 | Ar.0 | Ar.34 |
| A-1901 | Ar.1 | Ar.34 |
| A-1902 | Ar.2 | Ar.34 |
| A-1903 | Ar.3 | Ar.34 |
| A-1904 | Ar.4 | Ar.34 |
| A-1905 | Ar.5 | Ar.34 |
| A-1906 | Ar.6 | Ar.34 |
| A-1907 | Ar.7 | Ar.34 |
| A-1908 | Ar.8 | Ar.34 |
| A-1909 | Ar.9 | Ar.34 |
| A-1910 | Ar.10 | Ar.34 |
| A-1911 | Ar.11 | Ar.34 |
| A-1912 | Ar.12 | Ar.34 |
| A-1913 | Ar.13 | Ar.34 |
| A-1914 | Ar.14 | Ar.34 |
| A-1915 | Ar.15 | Ar.34 |
| A-1916 | CN | Ar.35 |
| A-1917 | OCH₃ | Ar.35 |
| A-1918 | SCH₃ | Ar.35 |
| A-1919 | OCF₃ | Ar.35 |
| A-1920 | OCHF₂ | Ar.35 |
| A-1921 | methyl | Ar.35 |
| A-1922 | iso-propyl | Ar.35 |
| A-1923 | ethenyl | Ar.35 |
| A-1924 | ethynyl | Ar.35 |
| A-1925 | Ar.0 | Ar.35 |
| A-1926 | Ar.1 | Ar.35 |
| A-1927 | Ar.2 | Ar.35 |
| A-1928 | Ar.3 | Ar.35 |
| A-1929 | Ar.4 | Ar.35 |
| A-1930 | Ar.5 | Ar.35 |
| A-1931 | Ar.6 | Ar.35 |
| A-1932 | Ar.7 | Ar.35 |
| A-1933 | Ar.8 | Ar.35 |
| A-1934 | Ar.9 | Ar.35 |
| A-1935 | Ar.10 | Ar.35 |
| A-1936 | Ar.11 | Ar.35 |
| A-1937 | Ar.12 | Ar.35 |
| A-1938 | Ar.13 | Ar.35 |
| A-1939 | Ar.14 | Ar.35 |
| A-1940 | Ar.15 | Ar.35 |
| A-1941 | CN | Ar.36 |
| A-1942 | OCH₃ | Ar.36 |
| A-1943 | SCH₃ | Ar.36 |
| A-1944 | OCF₃ | Ar.36 |
| A-1945 | OCHF₂ | Ar.36 |
| A-1946 | methyl | Ar.36 |
| A-1947 | iso-propyl | Ar.36 |
| A-1948 | ethenyl | Ar.36 |
| A-1949 | ethynyl | Ar.36 |
| A-1950 | Ar.0 | Ar.36 |
| A-1951 | Ar.1 | Ar.36 |
| A-1952 | Ar.2 | Ar.36 |
| A-1953 | Ar.3 | Ar.36 |
| A-1954 | Ar.4 | Ar.36 |
| A-1955 | Ar.5 | Ar.36 |
| A-1956 | Ar.6 | Ar.36 |
| A-1957 | Ar.7 | Ar.36 |
| A-1958 | Ar.8 | Ar.36 |
| A-1959 | Ar.9 | Ar.36 |
| A-1960 | Ar.10 | Ar.36 |
| A-1961 | Ar.11 | Ar.36 |
| A-1962 | Ar.12 | Ar.36 |
| A-1963 | Ar.13 | Ar.36 |
| A-1964 | Ar.14 | Ar.36 |
| A-1965 | Ar.15 | Ar.36 |
| A-1966 | CN | Ar.37 |
| A-1967 | OCH₃ | Ar.37 |
| A-1968 | SCH₃ | Ar.37 |
| A-1969 | OCF₃ | Ar.37 |
| A-1970 | OCHF₂ | Ar.37 |
| A-1971 | methyl | Ar.37 |
| A-1972 | iso-propyl | Ar.37 |
| A-1973 | ethenyl | Ar.37 |
| A-1974 | ethynyl | Ar.37 |
| A-1975 | Ar.0 | Ar.37 |
| A-1976 | Ar.1 | Ar.37 |
| A-1977 | Ar.2 | Ar.37 |
| A-1978 | Ar.3 | Ar.37 |
| A-1979 | Ar.4 | Ar.37 |
| A-1980 | Ar.5 | Ar.37 |
| A-1981 | Ar.6 | Ar.37 |
| A-1982 | Ar.7 | Ar.37 |
| A-1983 | Ar.8 | Ar.37 |
| A-1984 | Ar.9 | Ar.37 |
| A-1985 | Ar.10 | Ar.37 |
| A-1986 | Ar.11 | Ar.37 |
| A-1987 | Ar.12 | Ar.37 |
| A-1988 | Ar.13 | Ar.37 |
| A-1989 | Ar.14 | Ar.37 |
| A-1990 | Ar.15 | Ar.37 |
| A-1991 | CN | Ar.38 |
| A-1992 | OCH₃ | Ar.38 |
| A-1993 | SCH₃ | Ar.38 |
| A-1994 | OCF₃ | Ar.38 |
| A-1995 | OCHF₂ | Ar.38 |
| A-1996 | methyl | Ar.38 |
| A-1997 | iso-propyl | Ar.38 |
| A-1998 | ethenyl | Ar.38 |
| A-1999 | ethynyl | Ar.38 |
| A-2000 | Ar.0 | Ar.38 |
| A-2001 | Ar.1 | Ar.38 |
| A-2002 | Ar.2 | Ar.38 |
| A-2003 | Ar.3 | Ar.38 |
| A-2004 | Ar.4 | Ar.38 |
| A-2005 | Ar.5 | Ar.38 |
| A-2006 | Ar.6 | Ar.38 |
| A-2007 | Ar.7 | Ar.38 |
| A-2008 | Ar.8 | Ar.38 |
| A-2009 | Ar.9 | Ar.38 |
| A-2010 | Ar.10 | Ar.38 |
| A-2011 | Ar.11 | Ar.38 |
| A-2012 | Ar.12 | Ar.38 |
| A-2013 | Ar.13 | Ar.38 |
| A-2014 | Ar.14 | Ar.38 |
| A-2015 | Ar.15 | Ar.38 |
| A-2016 | CN | Ar.39 |
| A-2017 | OCH₃ | Ar.39 |
| A-2018 | SCH₃ | Ar.39 |
| A-2019 | OCF₃ | Ar.39 |
| A-2020 | OCHF₂ | Ar.39 |
| A-2021 | methyl | Ar.39 |
| A-2022 | iso-propyl | Ar.39 |
| A-2023 | ethenyl | Ar.39 |
| A-2024 | ethynyl | Ar.39 |
| A-2025 | Ar.0 | Ar.39 |
| A-2026 | Ar.1 | Ar.39 |
| A-2027 | Ar.2 | Ar.39 |
| A-2028 | Ar.3 | Ar.39 |
| A-2029 | Ar.4 | Ar.39 |
| A-2030 | Ar.5 | Ar.39 |
| A-2031 | Ar.6 | Ar.39 |
| A-2032 | Ar.7 | Ar.39 |
| A-2033 | Ar.8 | Ar.39 |
| A-2034 | Ar.9 | Ar.39 |
| A-2035 | Ar.10 | Ar.39 |
| A-2036 | Ar.11 | Ar.39 |
| A-2037 | Ar.12 | Ar.39 |
| A-2038 | Ar.13 | Ar.39 |
| A-2039 | Ar.14 | Ar.39 |
| A-2040 | Ar.15 | Ar.39 |
| A-2041 | CN | Ar.40 |
| A-2042 | OCH₃ | Ar.40 |
| A-2043 | SCH₃ | Ar.40 |
| A-2044 | OCF₃ | Ar.40 |
| A-2045 | OCHF₂ | Ar.40 |
| A-2046 | methyl | Ar.40 |
| A-2047 | iso-propyl | Ar.40 |
| A-2048 | ethenyl | Ar.40 |
| A-2049 | ethynyl | Ar.40 |
| A-2050 | Ar.0 | Ar.40 |
| A-2051 | Ar.1 | Ar.40 |
| A-2052 | Ar.2 | Ar.40 |
| A-2053 | Ar.3 | Ar.40 |
| A-2054 | Ar.4 | Ar.40 |
| A-2055 | Ar.5 | Ar.40 |
| A-2056 | Ar.6 | Ar.40 |
| A-2057 | Ar.7 | Ar.40 |
| A-2058 | Ar.8 | Ar.40 |
| A-2059 | Ar.9 | Ar.40 |
| A-2060 | Ar.10 | Ar.40 |
| A-2061 | Ar.11 | Ar.40 |
| A-2062 | Ar.12 | Ar.40 |
| A-2063 | Ar.13 | Ar.40 |
| A-2064 | Ar.14 | Ar.40 |
| A-2065 | Ar.15 | Ar.40 |
| A-2066 | CN | Ar.41 |
| A-2067 | OCH₃ | Ar.41 |
| A-2068 | SCH₃ | Ar.41 |
| A-2069 | OCF₃ | Ar.41 |
| A-2070 | OCHF₂ | Ar.41 |
| A-2071 | methyl | Ar.41 |
| A-2072 | iso-propyl | Ar.41 |
| A-2073 | ethenyl | Ar.41 |
| A-2074 | ethynyl | Ar.41 |
| A-2075 | Ar.0 | Ar.41 |
| A-2076 | Ar.1 | Ar.41 |
| A-2077 | Ar.2 | Ar.41 |
| A-2078 | Ar.3 | Ar.41 |
| A-2079 | Ar.4 | Ar.41 |
| A-2080 | Ar.5 | Ar.41 |
| A-2081 | Ar.6 | Ar.41 |
| A-2082 | Ar.7 | Ar.41 |
| A-2083 | Ar.8 | Ar.41 |
| A-2084 | Ar.9 | Ar.41 |
| A-2085 | Ar.10 | Ar.41 |
| A-2086 | Ar.11 | Ar.41 |
| A-2087 | Ar.12 | Ar.41 |
| A-2088 | Ar.13 | Ar.41 |
| A-2089 | Ar.14 | Ar.41 |
| A-2090 | Ar.15 | Ar.41 |
| A-2091 | CN | Ar.42 |
| A-2092 | OCH₃ | Ar.42 |
| A-2093 | SCH₃ | Ar.42 |
| A-2094 | OCF₃ | Ar.42 |
| A-2095 | OCHF₂ | Ar.42 |
| A-2096 | methyl | Ar.42 |
| A-2097 | iso-propyl | Ar.42 |
| A-2098 | ethenyl | Ar.42 |
| A-2099 | ethynyl | Ar.42 |
| A-2100 | Ar.0 | Ar.42 |
| A-2101 | Ar.1 | Ar.42 |
| A-2102 | Ar.2 | Ar.42 |
| A-2103 | Ar.3 | Ar.42 |
| A-2104 | Ar.4 | Ar.42 |
| A-2105 | Ar.5 | Ar.42 |
| A-2106 | Ar.6 | Ar.42 |
| A-2107 | Ar.7 | Ar.42 |
| A-2108 | Ar.8 | Ar.42 |
| A-2109 | Ar.9 | Ar.42 |
| A-2110 | Ar.10 | Ar.42 |
| A-2111 | Ar.11 | Ar.42 |
| A-2112 | Ar.12 | Ar.42 |
| A-2113 | Ar.13 | Ar.42 |
| A-2114 | Ar.14 | Ar.42 |
| A-2115 | Ar.15 | Ar.42 |
| A-2116 | CN | Ar.43 |
| A-2117 | OCH₃ | Ar.43 |
| A-2118 | SCH₃ | Ar.43 |
| A-2119 | OCF₃ | Ar.43 |
| A-2120 | OCHF₂ | Ar.43 |
| A-2121 | methyl | Ar.43 |
| A-2122 | iso-propyl | Ar.43 |
| A-2123 | ethenyl | Ar.43 |
| A-2124 | ethynyl | Ar.43 |
| A-2125 | Ar.0 | Ar.43 |
| A-2126 | Ar.1 | Ar.43 |
| A-2127 | Ar.2 | Ar.43 |
| A-2128 | Ar.3 | Ar.43 |
| A-2129 | Ar.4 | Ar.43 |
| A-2130 | Ar.5 | Ar.43 |
| A-2131 | Ar.6 | Ar.43 |
| A-2132 | Ar.7 | Ar.43 |
| A-2133 | Ar.8 | Ar.43 |
| A-2134 | Ar.9 | Ar.43 |
| A-2135 | Ar.10 | Ar.43 |
| A-2136 | Ar.11 | Ar.43 |
| A-2137 | Ar.12 | Ar.43 |
| A-2138 | Ar.13 | Ar.43 |
| A-2139 | Ar.14 | Ar.43 |
| A-2140 | Ar.15 | Ar.43 |
| A-2141 | CN | Ar.44 |
| A-2142 | OCH₃ | Ar.44 |
| A-2143 | SCH₃ | Ar.44 |
| A-2144 | OCF₃ | Ar.44 |
| A-2145 | OCHF₂ | Ar.44 |
| A-2146 | methyl | Ar.44 |
| A-2147 | iso-propyl | Ar.44 |
| A-2148 | ethenyl | Ar.44 |
| A-2149 | ethynyl | Ar.44 |
| A-2150 | Ar.0 | Ar.44 |
| A-2151 | Ar.1 | Ar.44 |
| A-2152 | Ar.2 | Ar.44 |
| A-2153 | Ar.3 | Ar.44 |
| A-2154 | Ar.4 | Ar.44 |
| A-2155 | Ar.5 | Ar.44 |
| A-2156 | Ar.6 | Ar.44 |
| A-2157 | Ar.7 | Ar.44 |
| A-2158 | Ar.8 | Ar.44 |
| A-2159 | Ar.9 | Ar.44 |
| A-2160 | Ar.10 | Ar.44 |
| A-2161 | Ar.11 | Ar.44 |
| A-2162 | Ar.12 | Ar.44 |
| A-2163 | Ar.13 | Ar.44 |
| A-2164 | Ar.14 | Ar.44 |
| A-2165 | Ar.15 | Ar.44 |
| A-2166 | CN | Ar.45 |
| A-2167 | OCH₃ | Ar.45 |
| A-2168 | SCH₃ | Ar.45 |
| A-2169 | OCF₃ | Ar.45 |
| A-2170 | OCHF₂ | Ar.45 |
| A-2171 | methyl | Ar.45 |
| A-2172 | iso-propyl | Ar.45 |
| A-2173 | ethenyl | Ar.45 |
| A-2174 | ethynyl | Ar.45 |
| A-2175 | Ar.0 | Ar.45 |
| A-2176 | Ar.1 | Ar.45 |
| A-2177 | Ar.2 | Ar.45 |
| A-2178 | Ar.3 | Ar.45 |
| A-2179 | Ar.4 | Ar.45 |
| A-2180 | Ar.5 | Ar.45 |
| A-2181 | Ar.6 | Ar.45 |
| A-2182 | Ar.7 | Ar.45 |
| A-2183 | Ar.8 | Ar.45 |
| A-2184 | Ar.9 | Ar.45 |
| A-2185 | Ar.10 | Ar.45 |
| A-2186 | Ar.11 | Ar.45 |
| A-2187 | Ar.12 | Ar.45 |
| A-2188 | Ar.13 | Ar.45 |
| A-2189 | Ar.14 | Ar.45 |
| A-2190 | Ar.15 | Ar.45 |
| A-2191 | CN | Ar.46 |
| A-2192 | OCH₃ | Ar.46 |
| A-2193 | SCH₃ | Ar.46 |
| A-2194 | OCF₃ | Ar.46 |
| A-2195 | OCHF₂ | Ar.46 |
| A-2196 | methyl | Ar.46 |
| A-2197 | iso-propyl | Ar.46 |
| A-2198 | ethenyl | Ar.46 |
| A-2199 | ethynyl | Ar.46 |
| A-2200 | Ar.0 | Ar.46 |
| A-2201 | Ar.1 | Ar.46 |
| A-2202 | Ar.2 | Ar.46 |
| A-2203 | Ar.3 | Ar.46 |
| A-2204 | Ar.4 | Ar.46 |
| A-2205 | Ar.5 | Ar.46 |
| A-2206 | Ar.6 | Ar.46 |
| A-2207 | Ar.7 | Ar.46 |
| A-2208 | Ar.8 | Ar.46 |
| A-2209 | Ar.9 | Ar.46 |
| A-2210 | Ar.10 | Ar.46 |
| A-2211 | Ar.11 | Ar.46 |
| A-2212 | Ar.12 | Ar.46 |
| A-2213 | Ar.13 | Ar.46 |
| A-2214 | Ar.14 | Ar.46 |
| A-2215 | Ar.15 | Ar.46 |
| A-2216 | CN | Ar.47 |
| A-2217 | OCH₃ | Ar.47 |
| A-2218 | SCH₃ | Ar.47 |
| A-2219 | OCF₃ | Ar.47 |
| A-2220 | OCHF₂ | Ar.47 |
| A-2221 | methyl | Ar.47 |
| A-2222 | iso-propyl | Ar.47 |
| A-2223 | ethenyl | Ar.47 |
| A-2224 | ethynyl | Ar.47 |
| A-2225 | Ar.0 | Ar.47 |
| A-2226 | Ar.1 | Ar.47 |
| A-2227 | Ar.2 | Ar.47 |
| A-2228 | Ar.3 | Ar.47 |
| A-2229 | Ar.4 | Ar.47 |
| A-2230 | Ar.5 | Ar.47 |
| A-2231 | Ar.6 | Ar.47 |
| A-2232 | Ar.7 | Ar.47 |
| A-2233 | Ar.8 | Ar.47 |
| A-2234 | Ar.9 | Ar.47 |
| A-2235 | Ar.10 | Ar.47 |
| A-2236 | Ar.11 | Ar.47 |
| A-2237 | Ar.12 | Ar.47 |
| A-2238 | Ar.13 | Ar.47 |
| A-2239 | Ar.14 | Ar.47 |
| A-2240 | Ar.15 | Ar.47 |
| A-2241 | CN | Ar.48 |
| A-2242 | OCH₃ | Ar.48 |
| A-2243 | SCH₃ | Ar.48 |
| A-2244 | OCF₃ | Ar.48 |
| A-2245 | OCHF₂ | Ar.48 |
| A-2246 | methyl | Ar.48 |
| A-2247 | iso-propyl | Ar.48 |
| A-2248 | ethenyl | Ar.48 |
| A-2249 | ethynyl | Ar.48 |
| A-2250 | Ar.0 | Ar.48 |
| A-2251 | Ar.1 | Ar.48 |
| A-2252 | Ar.2 | Ar.48 |
| A-2253 | Ar.3 | Ar.48 |
| A-2254 | Ar.4 | Ar.48 |
| A-2255 | Ar.5 | Ar.48 |
| A-2256 | Ar.6 | Ar.48 |
| A-2257 | Ar.7 | Ar.48 |
| A-2258 | Ar.8 | Ar.48 |
| A-2259 | Ar.9 | Ar.48 |
| A-2260 | Ar.10 | Ar.48 |
| A-2261 | Ar.11 | Ar.48 |
| A-2262 | Ar.12 | Ar.48 |
| A-2263 | Ar.13 | Ar.48 |
| A-2264 | Ar.14 | Ar.48 |
| A-2265 | Ar.15 | Ar.48 |
| A-2266 | CN | Ar.49 |
| A-2267 | OCH₃ | Ar.49 |
| A-2268 | SCH₃ | Ar.49 |
| A-2269 | OCF₃ | Ar.49 |
| A-2270 | OCHF₂ | Ar.49 |
| A-2271 | methyl | Ar.49 |
| A-2272 | iso-propyl | Ar.49 |
| A-2273 | ethenyl | Ar.49 |
| A-2274 | ethynyl | Ar.49 |
| A-2275 | Ar.0 | Ar.49 |
| A-2276 | Ar.1 | Ar.49 |
| A-2277 | Ar.2 | Ar.49 |
| A-2278 | Ar.3 | Ar.49 |
| A-2279 | Ar.4 | Ar.49 |
| A-2280 | Ar.5 | Ar.49 |
| A-2281 | Ar.6 | Ar.49 |
| A-2282 | Ar.7 | Ar.49 |
| A-2283 | Ar.8 | Ar.49 |
| A-2284 | Ar.9 | Ar.49 |
| A-2285 | Ar.10 | Ar.49 |
| A-2286 | Ar.11 | Ar.49 |
| A-2287 | Ar.12 | Ar.49 |
| A-2288 | Ar.13 | Ar.49 |
| A-2289 | Ar.14 | Ar.49 |
| A-2290 | Ar.15 | Ar.49 |
| A-2291 | CN | Ar.50 |
| A-2292 | OCH₃ | Ar.50 |
| A-2293 | SCH₃ | Ar.50 |
| A-2294 | OCF₃ | Ar.50 |
| A-2295 | OCHF₂ | Ar.50 |
| A-2296 | methyl | Ar.50 |
| A-2297 | iso-propyl | Ar.50 |
| A-2298 | ethenyl | Ar.50 |
| A-2299 | ethynyl | Ar.50 |
| A-2300 | Ar.0 | Ar.50 |
| A-2301 | Ar.1 | Ar.50 |
| A-2302 | Ar.2 | Ar.50 |
| A-2303 | Ar.3 | Ar.50 |
| A-2304 | Ar.4 | Ar.50 |
| A-2305 | Ar.5 | Ar.50 |
| A-2306 | Ar.6 | Ar.50 |
| A-2307 | Ar.7 | Ar.50 |
| A-2308 | Ar.8 | Ar.50 |
| A-2309 | Ar.9 | Ar.50 |
| A-2310 | Ar.10 | Ar.50 |
| A-2311 | Ar.11 | Ar.50 |
| A-2312 | Ar.12 | Ar.50 |
| A-2313 | Ar.13 | Ar.50 |
| A-2314 | Ar.14 | Ar.50 |
| A-2315 | Ar.15 | Ar.50 |
| A-2316 | CN | Ar.51 |
| A-2317 | OCH₃ | Ar.51 |
| A-2318 | SCH₃ | Ar.51 |
| A-2319 | OCF₃ | Ar.51 |
| A-2320 | OCHF₂ | Ar.51 |
| A-2321 | methyl | Ar.51 |
| A-2322 | iso-propyl | Ar.51 |
| A-2323 | ethenyl | Ar.51 |
| A-2324 | ethynyl | Ar.51 |
| A-2325 | Ar.0 | Ar.51 |
| A-2326 | Ar.1 | Ar.51 |
| A-2327 | Ar.2 | Ar.51 |
| A-2328 | Ar.3 | Ar.51 |
| A-2329 | Ar.4 | Ar.51 |
| A-2330 | Ar.5 | Ar.51 |
| A-2331 | Ar.6 | Ar.51 |
| A-2332 | Ar.7 | Ar.51 |
| A-2333 | Ar.8 | Ar.51 |
| A-2334 | Ar.9 | Ar.51 |
| A-2335 | Ar.10 | Ar.51 |
| A-2336 | Ar.11 | Ar.51 |
| A-2337 | Ar.12 | Ar.51 |
| A-2338 | Ar.13 | Ar.51 |
| A-2339 | Ar.14 | Ar.51 |
| A-2340 | Ar.15 | Ar.51 |
| A-2341 | CN | Ar.52 |
| A-2342 | OCH₃ | Ar.52 |
| A-2343 | SCH₃ | Ar.52 |
| A-2344 | OCF₃ | Ar.52 |
| A-2345 | OCHF₂ | Ar.52 |
| A-2346 | methyl | Ar.52 |
| A-2347 | iso-propyl | Ar.52 |
| A-2348 | ethenyl | Ar.52 |
| A-2349 | ethynyl | Ar.52 |
| A-2350 | Ar.0 | Ar.52 |
| A-2351 | Ar.1 | Ar.52 |
| A-2352 | Ar.2 | Ar.52 |
| A-2353 | Ar.3 | Ar.52 |
| A-2354 | Ar.4 | Ar.52 |
| A-2355 | Ar.5 | Ar.52 |
| A-2356 | Ar.6 | Ar.52 |
| A-2357 | Ar.7 | Ar.52 |
| A-2358 | Ar.8 | Ar.52 |
| A-2359 | Ar.9 | Ar.52 |
| A-2360 | Ar.10 | Ar.52 |
| A-2361 | Ar.11 | Ar.52 |
| A-2362 | Ar.12 | Ar.52 |
| A-2363 | Ar.13 | Ar.52 |
| A-2364 | Ar.14 | Ar.52 |
| A-2365 | Ar.15 | Ar.52 |
| A-2366 | CN | Ar.53 |
| A-2367 | OCH₃ | Ar.53 |
| A-2368 | SCH₃ | Ar.53 |
| A-2369 | OCF₃ | Ar.53 |
| A-2370 | OCHF₂ | Ar.53 |
| A-2371 | methyl | Ar.53 |
| A-2372 | iso-propyl | Ar.53 |
| A-2373 | ethenyl | Ar.53 |
| A-2374 | ethynyl | Ar.53 |
| A-2375 | Ar.0 | Ar.53 |
| A-2376 | Ar.1 | Ar.53 |
| A-2377 | Ar.2 | Ar.53 |
| A-2378 | Ar.3 | Ar.53 |
| A-2379 | Ar.4 | Ar.53 |
| A-2380 | Ar.5 | Ar.53 |
| A-2381 | Ar.6 | Ar.53 |
| A-2382 | Ar.7 | Ar.53 |
| A-2383 | Ar.8 | Ar.53 |
| A-2384 | Ar.9 | Ar.53 |
| A-2385 | Ar.10 | Ar.53 |
| A-2386 | Ar.11 | Ar.53 |
| A-2387 | Ar.12 | Ar.53 |
| A-2388 | Ar.13 | Ar.53 |
| A-2389 | Ar.14 | Ar.53 |
| A-2390 | Ar.15 | Ar.53 |
| A-2391 | CN | Ar.54 |
| A-2392 | OCH₃ | Ar.54 |
| A-2393 | SCH₃ | Ar.54 |
| A-2394 | OCF₃ | Ar.54 |
| A-2395 | OCHF₂ | Ar.54 |
| A-2396 | methyl | Ar.54 |
| A-2397 | iso-propyl | Ar.54 |
| A-2398 | ethenyl | Ar.54 |
| A-2399 | ethynyl | Ar.54 |
| A-2400 | Ar.0 | Ar.54 |
| A-2401 | Ar.1 | Ar.54 |
| A-2402 | Ar.2 | Ar.54 |
| A-2403 | Ar.3 | Ar.54 |
| A-2404 | Ar.4 | Ar.54 |
| A-2405 | Ar.5 | Ar.54 |
| A-2406 | Ar.6 | Ar.54 |
| A-2407 | Ar.7 | Ar.54 |
| A-2408 | Ar.8 | Ar.54 |
| A-2409 | Ar.9 | Ar.54 |
| A-2410 | Ar.10 | Ar.54 |
| A-2411 | Ar.11 | Ar.54 |
| A-2412 | Ar.12 | Ar.54 |
| A-2413 | Ar.13 | Ar.54 |
| A-2414 | Ar.14 | Ar.54 |
| A-2415 | Ar.15 | Ar.54 |
| A-2416 | CN | Ar.55 |
| A-2417 | OCH₃ | Ar.55 |
| A-2418 | SCH₃ | Ar.55 |
| A-2419 | OCF₃ | Ar.55 |
| A-2420 | OCHF₂ | Ar.55 |
| A-2421 | methyl | Ar.55 |
| A-2422 | iso-propyl | Ar.55 |
| A-2423 | ethenyl | Ar.55 |
| A-2424 | ethynyl | Ar.55 |
| A-2425 | Ar.0 | Ar.55 |
| A-2426 | Ar.1 | Ar.55 |
| A-2427 | Ar.2 | Ar.55 |
| A-2428 | Ar.3 | Ar.55 |
| A-2429 | Ar.4 | Ar.55 |
| A-2430 | Ar.5 | Ar.55 |
| A-2431 | Ar.6 | Ar.55 |
| A-2432 | Ar.7 | Ar.55 |
| A-2433 | Ar.8 | Ar.55 |
| A-2434 | Ar.9 | Ar.55 |
| A-2435 | Ar.10 | Ar.55 |
| A-2436 | Ar.11 | Ar.55 |
| A-2437 | Ar.12 | Ar.55 |
| A-2438 | Ar.13 | Ar.55 |
| A-2439 | Ar.14 | Ar.55 |
| A-2440 | Ar.15 | Ar.55 |
| A-2441 | CN | Ar.56 |
| A-2442 | OCH₃ | Ar.56 |
| A-2443 | SCH₃ | Ar.56 |
| A-2444 | OCF₃ | Ar.56 |
| A-2445 | OCHF₂ | Ar.56 |
| A-2446 | methyl | Ar.56 |
| A-2447 | iso-propyl | Ar.56 |
| A-2448 | ethenyl | Ar.56 |
| A-2449 | ethynyl | Ar.56 |
| A-2450 | Ar.0 | Ar.56 |
| A-2451 | Ar.1 | Ar.56 |
| A-2452 | Ar.2 | Ar.56 |
| A-2453 | Ar.3 | Ar.56 |
| A-2454 | Ar.4 | Ar.56 |
| A-2455 | Ar.5 | Ar.56 |
| A-2456 | Ar.6 | Ar.56 |
| A-2457 | Ar.7 | Ar.56 |
| A-2458 | Ar.8 | Ar.56 |
| A-2459 | Ar.9 | Ar.56 |
| A-2460 | Ar.10 | Ar.56 |
| A-2461 | Ar.11 | Ar.56 |
| A-2462 | Ar.12 | Ar.56 |
| A-2463 | Ar.13 | Ar.56 |
| A-2464 | Ar.14 | Ar.56 |
| A-2465 | Ar.15 | Ar.56 |
| A-2466 | CN | Ar.57 |
| A-2467 | OCH₃ | Ar.57 |
| A-2468 | SCH₃ | Ar.57 |
| A-2469 | OCF₃ | Ar.57 |
| A-2470 | OCHF₂ | Ar.57 |
| A-2471 | methyl | Ar.57 |
| A-2472 | iso-propyl | Ar.57 |
| A-2473 | ethenyl | Ar.57 |
| A-2474 | ethynyl | Ar.57 |
| A-2475 | Ar.0 | Ar.57 |
| A-2476 | Ar.1 | Ar.57 |
| A-2477 | Ar.2 | Ar.57 |
| A-2478 | Ar.3 | Ar.57 |
| A-2479 | Ar.4 | Ar.57 |
| A-2480 | Ar.5 | Ar.57 |
| A-2481 | Ar.6 | Ar.57 |
| A-2482 | Ar.7 | Ar.57 |
| A-2483 | Ar.8 | Ar.57 |
| A-2484 | Ar.9 | Ar.57 |
| A-2485 | Ar.10 | Ar.57 |
| A-2486 | Ar.11 | Ar.57 |
| A-2487 | Ar.12 | Ar.57 |
| A-2488 | Ar.13 | Ar.57 |
| A-2489 | Ar.14 | Ar.57 |
| A-2490 | Ar.15 | Ar.57 |
| A-2491 | CN | Ar.58 |
| A-2492 | OCH₃ | Ar.58 |
| A-2493 | SCH₃ | Ar.58 |
| A-2494 | OCF₃ | Ar.58 |
| A-2495 | OCHF₂ | Ar.58 |
| A-2496 | methyl | Ar.58 |
| A-2497 | iso-propyl | Ar.58 |
| A-2498 | ethenyl | Ar.58 |
| A-2499 | ethynyl | Ar.58 |
| A-2500 | Ar.0 | Ar.58 |
| A-2501 | Ar.1 | Ar.58 |
| A-2502 | Ar.2 | Ar.58 |
| A-2503 | Ar.3 | Ar.58 |
| A-2504 | Ar.4 | Ar.58 |
| A-2505 | Ar.5 | Ar.58 |
| A-2506 | Ar.6 | Ar.58 |
| A-2507 | Ar.7 | Ar.58 |
| A-2508 | Ar.8 | Ar.58 |
| A-2509 | Ar.9 | Ar.58 |
| A-2510 | Ar.10 | Ar.58 |
| A-2511 | Ar.11 | Ar.58 |
| A-2512 | Ar.12 | Ar.58 |
| A-2513 | Ar.13 | Ar.58 |
| A-2514 | Ar.14 | Ar.58 |
| A-2515 | Ar.15 | Ar.58 |
| A-2516 | CN | Ar.59 |
| A-2517 | OCH₃ | Ar.59 |
| A-2518 | SCH₃ | Ar.59 |
| A-2519 | OCF₃ | Ar.59 |
| A-2520 | OCHF₂ | Ar.59 |
| A-2521 | methyl | Ar.59 |
| A-2522 | iso-propyl | Ar.59 |
| A-2523 | ethenyl | Ar.59 |
| A-2524 | ethynyl | Ar.59 |
| A-2525 | Ar.0 | Ar.59 |
| A-2526 | Ar.1 | Ar.59 |
| A-2527 | Ar.2 | Ar.59 |
| A-2528 | Ar.3 | Ar.59 |
| A-2529 | Ar.4 | Ar.59 |
| A-2530 | Ar.5 | Ar.59 |
| A-2531 | Ar.6 | Ar.59 |
| A-2532 | Ar.7 | Ar.59 |
| A-2533 | Ar.8 | Ar.59 |
| A-2534 | Ar.9 | Ar.59 |
| A-2535 | Ar.10 | Ar.59 |
| A-2536 | Ar.11 | Ar.59 |
| A-2537 | Ar.12 | Ar.59 |
| A-2538 | Ar.13 | Ar.59 |
| A-2539 | Ar.14 | Ar.59 |
| A-2540 | Ar.15 | Ar.59 |
| A-2541 | CN | Ar.60 |
| A-2542 | OCH₃ | Ar.60 |
| A-2543 | SCH₃ | Ar.60 |
| A-2544 | OCF₃ | Ar.60 |
| A-2545 | OCHF₂ | Ar.60 |
| A-2546 | methyl | Ar.60 |
| A-2547 | iso-propyl | Ar.60 |
| A-2548 | ethenyl | Ar.60 |
| A-2549 | ethynyl | Ar.60 |
| A-2550 | Ar.0 | Ar.60 |
| A-2551 | Ar.1 | Ar.60 |
| A-2552 | Ar.2 | Ar.60 |
| A-2553 | Ar.3 | Ar.60 |
| A-2554 | Ar.4 | Ar.60 |
| A-2555 | Ar.5 | Ar.60 |
| A-2556 | Ar.6 | Ar.60 |
| A-2557 | Ar.7 | Ar.60 |
| A-2558 | Ar.8 | Ar.60 |
| A-2559 | Ar.9 | Ar.60 |
| A-2560 | Ar.10 | Ar.60 |
| A-2561 | Ar.11 | Ar.60 |
| A-2562 | Ar.12 | Ar.60 |
| A-2563 | Ar.13 | Ar.60 |
| A-2564 | Ar.14 | Ar.60 |
| A-2565 | Ar.15 | Ar.60 |
| A-2566 | CN | Ar.61 |
| A-2567 | OCH₃ | Ar.61 |
| A-2568 | SCH₃ | Ar.61 |
| A-2569 | OCF₃ | Ar.61 |
| A-2570 | OCHF₂ | Ar.61 |
| A-2571 | methyl | Ar.61 |
| A-2572 | iso-propyl | Ar.61 |
| A-2573 | ethenyl | Ar.61 |
| A-2574 | ethynyl | Ar.61 |
| A-2575 | Ar.0 | Ar.61 |
| A-2576 | Ar.1 | Ar.61 |
| A-2577 | Ar.2 | Ar.61 |
| A-2578 | Ar.3 | Ar.61 |
| A-2579 | Ar.4 | Ar.61 |
| A-2580 | Ar.5 | Ar.61 |
| A-2581 | Ar.6 | Ar.61 |
| A-2582 | Ar.7 | Ar.61 |
| A-2583 | Ar.8 | Ar.61 |
| A-2584 | Ar.9 | Ar.61 |
| A-2585 | Ar.10 | Ar.61 |
| A-2586 | Ar.11 | Ar.61 |
| A-2587 | Ar.12 | Ar.61 |
| A-2588 | Ar.13 | Ar.61 |
| A-2589 | Ar.14 | Ar.61 |
| A-2590 | Ar.15 | Ar.61 |
| A-2591 | CN | Ar.62 |
| A-2592 | OCH₃ | Ar.62 |
| A-2593 | SCH₃ | Ar.62 |
| A-2594 | OCF₃ | Ar.62 |
| A-2595 | OCHF₂ | Ar.62 |
| A-2596 | methyl | Ar.62 |
| A-2597 | iso-propyl | Ar.62 |
| A-2598 | ethenyl | Ar.62 |
| A-2599 | ethynyl | Ar.62 |
| A-2600 | Ar.0 | Ar.62 |
| A-2601 | Ar.1 | Ar.62 |
| A-2602 | Ar.2 | Ar.62 |
| A-2603 | Ar.3 | Ar.62 |
| A-2604 | Ar.4 | Ar.62 |
| A-2605 | Ar.5 | Ar.62 |
| A-2606 | Ar.6 | Ar.62 |
| A-2607 | Ar.7 | Ar.62 |
| A-2608 | Ar.8 | Ar.62 |
| A-2609 | Ar.9 | Ar.62 |
| A-2610 | Ar.10 | Ar.62 |
| A-2611 | Ar.11 | Ar.62 |
| A-2612 | Ar.12 | Ar.62 |
| A-2613 | Ar.13 | Ar.62 |
| A-2614 | Ar.14 | Ar.62 |
| A-2615 | Ar.15 | Ar.62 |
| A-2616 | CN | Ar.63 |
| A-2617 | OCH₃ | Ar.63 |
| A-2618 | SCH₃ | Ar.63 |
| A-2619 | OCF₃ | Ar.63 |
| A-2620 | OCHF₂ | Ar.63 |
| A-2621 | methyl | Ar.63 |
| A-2622 | iso-propyl | Ar.63 |
| A-2623 | ethenyl | Ar.63 |
| A-2624 | ethynyl | Ar.63 |
| A-2625 | Ar.0 | Ar.63 |
| A-2626 | Ar.1 | Ar.63 |
| A-2627 | Ar.2 | Ar.63 |
| A-2628 | Ar.3 | Ar.63 |
| A-2629 | Ar.4 | Ar.63 |
| A-2630 | Ar.5 | Ar.63 |
| A-2631 | Ar.6 | Ar.63 |
| A-2632 | Ar.7 | Ar.63 |
| A-2633 | Ar.8 | Ar.63 |
| A-2634 | Ar.9 | Ar.63 |
| A-2635 | Ar.10 | Ar.63 |
| A-2636 | Ar.11 | Ar.63 |
| A-2637 | Ar.12 | Ar.63 |
| A-2638 | Ar.13 | Ar.63 |
| A-2639 | Ar.14 | Ar.63 |
| A-2640 | Ar.15 | Ar.63 |
| A-2641 | CN | Ar.64 |
| A-2642 | OCH₃ | Ar.64 |
| A-2643 | SCH₃ | Ar.64 |
| A-2644 | OCF₃ | Ar.64 |
| A-2645 | OCHF₂ | Ar.64 |
| A-2646 | methyl | Ar.64 |
| A-2647 | iso-propyl | Ar.64 |
| A-2648 | ethenyl | Ar.64 |
| A-2649 | ethynyl | Ar.64 |
| A-2650 | Ar.0 | Ar.64 |
| A-2651 | Ar.1 | Ar.64 |
| A-2652 | Ar.2 | Ar.64 |
| A-2653 | Ar.3 | Ar.64 |
| A-2654 | Ar.4 | Ar.64 |
| A-2655 | Ar.5 | Ar.64 |
| A-2656 | Ar.6 | Ar.64 |
| A-2657 | Ar.7 | Ar.64 |
| A-2658 | Ar.8 | Ar.64 |
| A-2659 | Ar.9 | Ar.64 |
| A-2660 | Ar.10 | Ar.64 |
| A-2661 | Ar.11 | Ar.64 |
| A-2662 | Ar.12 | Ar.64 |
| A-2663 | Ar.13 | Ar.64 |
| A-2664 | Ar.14 | Ar.64 |
| A-2665 | Ar.15 | Ar.64 |
| A-2666 | CN | Ar.65 |
| A-2667 | OCH₃ | Ar.65 |
| A-2668 | SCH₃ | Ar.65 |
| A-2669 | OCF₃ | Ar.65 |
| A-2670 | OCHF₂ | Ar.65 |
| A-2671 | methyl | Ar.65 |
| A-2672 | iso-propyl | Ar.65 |
| A-2673 | ethenyl | Ar.65 |
| A-2674 | ethynyl | Ar.65 |
| A-2675 | Ar.0 | Ar.65 |
| A-2676 | Ar.1 | Ar.65 |
| A-2677 | Ar.2 | Ar.65 |
| A-2678 | Ar.3 | Ar.65 |
| A-2679 | Ar.4 | Ar.65 |
| A-2680 | Ar.5 | Ar.65 |
| A-2681 | Ar.6 | Ar.65 |
| A-2682 | Ar.7 | Ar.65 |
| A-2683 | Ar.8 | Ar.65 |
| A-2684 | Ar.9 | Ar.65 |
| A-2685 | Ar.10 | Ar.65 |
| A-2686 | Ar.11 | Ar.65 |
| A-2687 | Ar.12 | Ar.65 |
| A-2688 | Ar.13 | Ar.65 |
| A-2689 | Ar.14 | Ar.65 |
| A-2690 | Ar.15 | Ar.65 |
| A-2691 | OCH₃ | Ar.66 |
| A-2692 | SCH₃ | Ar.66 |
| A-2693 | OCF₃ | Ar.66 |
| A-2694 | OCHF₂ | Ar.66 |
| A-2695 | methyl | Ar.66 |
| A-2696 | iso-propyl | Ar.66 |
| A-2697 | ethenyl | Ar.66 |
| A-2698 | ethynyl | Ar.66 |
| A-2699 | Ar.0 | Ar.66 |
| A-2700 | Ar.1 | Ar.66 |
| A-2701 | Ar.2 | Ar.66 |
| A-2702 | Ar.3 | Ar.66 |
| A-2703 | Ar.4 | Ar.66 |
| A-2704 | Ar.5 | Ar.66 |
| A-2705 | Ar.6 | Ar.66 |
| A-2706 | Ar.7 | Ar.66 |
| A-2707 | Ar.8 | Ar.66 |
| A-2708 | Ar.9 | Ar.66 |
| A-2709 | Ar.10 | Ar.66 |
| A-2710 | Ar.11 | Ar.66 |
| A-2711 | Ar.12 | Ar.66 |
| A-2712 | Ar.13 | Ar.66 |
| A-2713 | Ar.14 | Ar.66 |
| A-2714 | Ar.15 | Ar.66 |
| A-2715 | OCH₃ | Ar.67 |
| A-2716 | SCH₃ | Ar.67 |
| A-2717 | OCF₃ | Ar.67 |
| A-2718 | OCHF₂ | Ar.67 |
| A-2719 | methyl | Ar.67 |
| A-2720 | iso-propyl | Ar.67 |
| A-2721 | ethenyl | Ar.67 |
| A-2722 | ethynyl | Ar.67 |
| A-2723 | Ar.0 | Ar.67 |
| A-2724 | Ar.1 | Ar.67 |
| A-2725 | Ar.2 | Ar.67 |
| A-2726 | Ar.3 | Ar.67 |
| A-2727 | Ar.4 | Ar.67 |
| A-2728 | Ar.5 | Ar.67 |
| A-2729 | Ar.6 | Ar.67 |
| A-2730 | Ar.7 | Ar.67 |
| A-2731 | Ar.8 | Ar.67 |
| A-2732 | Ar.9 | Ar.67 |
| A-2733 | Ar.10 | Ar.67 |
| A-2734 | Ar.11 | Ar.67 |
| A-2735 | Ar.12 | Ar.67 |
| A-2736 | Ar.13 | Ar.67 |
| A-2737 | Ar.14 | Ar.67 |
| A-2738 | Ar.15 | Ar.67 |
| A-2739 | OCH₃ | Ar.68 |
| A-2740 | SCH₃ | Ar.68 |
| A-2741 | OCF₃ | Ar.68 |
| A-2742 | OCHF₂ | Ar.68 |
| A-2743 | methyl | Ar.68 |
| A-2744 | iso-propyl | Ar.68 |
| A-2745 | ethenyl | Ar.68 |
| A-2746 | ethynyl | Ar.68 |
| A-2747 | Ar.0 | Ar.68 |
| A-2748 | Ar.1 | Ar.68 |
| A-2749 | Ar.2 | Ar.68 |
| A-2750 | Ar.3 | Ar.68 |
| A-2751 | Ar.4 | Ar.68 |
| A-2752 | Ar.5 | Ar.68 |
| A-2753 | Ar.6 | Ar.68 |
| A-2754 | Ar.7 | Ar.68 |
| A-2755 | Ar.8 | Ar.68 |
| A-2756 | Ar.9 | Ar.68 |
| A-2757 | Ar.10 | Ar.68 |
| A-2758 | Ar.11 | Ar.68 |
| A-2759 | Ar.12 | Ar.68 |
| A-2760 | Ar.13 | Ar.68 |
| A-2761 | Ar.14 | Ar.68 |
| A-2762 | Ar.15 | Ar.68 |
| A-2763 | OCH₃ | Ar.69 |
| A-2764 | SCH₃ | Ar.69 |
| A-2765 | OCF₃ | Ar.69 |
| A-2766 | OCHF₂ | Ar.69 |
| A-2767 | methyl | Ar.69 |
| A-2768 | iso-propyl | Ar.69 |
| A-2769 | ethenyl | Ar.69 |
| A-2770 | ethynyl | Ar.69 |
| A-2771 | Ar.0 | Ar.69 |
| A-2772 | Ar.1 | Ar.69 |
| A-2773 | Ar.2 | Ar.69 |
| A-2774 | Ar.3 | Ar.69 |
| A-2775 | Ar.4 | Ar.69 |
| A-2776 | Ar.5 | Ar.69 |
| A-2777 | Ar.6 | Ar.69 |
| A-2778 | Ar.7 | Ar.69 |
| A-2779 | Ar.8 | Ar.69 |
| A-2780 | Ar.9 | Ar.69 |
| A-2781 | Ar.10 | Ar.69 |
| A-2782 | Ar.11 | Ar.69 |
| A-2783 | Ar.12 | Ar.69 |
| A-2784 | Ar.13 | Ar.69 |
| A-2785 | Ar.14 | Ar.69 |
| A-2786 | Ar.15 | Ar.69 |
| A-2787 | OCH₃ | Ar.70 |
| A-2788 | SCH₃ | Ar.70 |
| A-2789 | OCF₃ | Ar.70 |
| A-2790 | OCHF₂ | Ar.70 |
| A-2791 | methyl | Ar.70 |
| A-2792 | iso-propyl | Ar.70 |
| A-2793 | ethenyl | Ar.70 |
| A-2794 | ethynyl | Ar.70 |
| A-2795 | Ar.0 | Ar.70 |
| A-2796 | Ar.1 | Ar.70 |
| A-2797 | Ar.2 | Ar.70 |
| A-2798 | Ar.3 | Ar.70 |
| A-2799 | Ar.4 | Ar.70 |
| A-2800 | Ar.5 | Ar.70 |
| A-2801 | Ar.6 | Ar.70 |
| A-2802 | Ar.7 | Ar.70 |
| A-2803 | Ar.8 | Ar.70 |
| A-2804 | Ar.9 | Ar.70 |
| A-2805 | Ar.10 | Ar.70 |
| A-2806 | Ar.11 | Ar.70 |
| A-2807 | Ar.12 | Ar.70 |
| A-2808 | Ar.13 | Ar.70 |
| A-2809 | Ar.14 | Ar.70 |
| A-2810 | Ar.15 | Ar.70 |
| A-2811 | OCH₃ | Ar.71 |
| A-2812 | SCH₃ | Ar.71 |
| A-2813 | OCF₃ | Ar.71 |
| A-2814 | OCHF₂ | Ar.71 |
| A-2815 | methyl | Ar.71 |
| A-2816 | iso-propyl | Ar.71 |
| A-2817 | ethenyl | Ar.71 |
| A-2818 | ethynyl | Ar.71 |
| A-2819 | Ar.0 | Ar.71 |
| A-2820 | Ar.1 | Ar.71 |
| A-2821 | Ar.2 | Ar.71 |
| A-2822 | Ar.3 | Ar.71 |
| A-2823 | Ar.4 | Ar.71 |
| A-2824 | Ar.5 | Ar.71 |
| A-2825 | Ar.6 | Ar.71 |
| A-2826 | Ar.7 | Ar.71 |
| A-2827 | Ar.8 | Ar.71 |
| A-2828 | Ar.9 | Ar.71 |
| A-2829 | Ar.10 | Ar.71 |
| A-2830 | Ar.11 | Ar.71 |
| A-2831 | Ar.12 | Ar.71 |
| A-2832 | Ar.13 | Ar.71 |
| A-2833 | Ar.14 | Ar.71 |
| A-2834 | Ar.15 | Ar.71 |
| A-2835 | OCH₃ | Ar.72 |
| A-2836 | SCH₃ | Ar.72 |
| A-2837 | OCF₃ | Ar.72 |
| A-2838 | OCHF₂ | Ar.72 |
| A-2839 | methyl | Ar.72 |
| A-2840 | iso-propyl | Ar.72 |
| A-2841 | ethenyl | Ar.72 |
| A-2842 | ethynyl | Ar.72 |
| A-2843 | Ar.0 | Ar.72 |
| A-2844 | Ar.1 | Ar.72 |
| A-2845 | Ar.2 | Ar.72 |
| A-2846 | Ar.3 | Ar.72 |
| A-2847 | Ar.4 | Ar.72 |
| A-2848 | Ar.5 | Ar.72 |
| A-2849 | Ar.6 | Ar.72 |
| A-2850 | Ar.7 | Ar.72 |
| A-2851 | Ar.8 | Ar.72 |
| A-2852 | Ar.9 | Ar.72 |
| A-2853 | Ar.10 | Ar.72 |
| A-2854 | Ar.11 | Ar.72 |
| A-2855 | Ar.12 | Ar.72 |
| A-2856 | Ar.13 | Ar.72 |
| A-2857 | Ar.14 | Ar.72 |
| A-2858 | Ar.15 | Ar.72 |
| A-2859 | OCH₃ | Ar.73 |
| A-2860 | SCH₃ | Ar.73 |
| A-2861 | OCF₃ | Ar.73 |
| A-2862 | OCHF₂ | Ar.73 |
| A-2863 | methyl | Ar.73 |
| A-2864 | iso-propyl | Ar.73 |
| A-2865 | ethenyl | Ar.73 |
| A-2866 | ethynyl | Ar.73 |
| A-2867 | Ar.0 | Ar.73 |
| A-2868 | Ar.1 | Ar.73 |
| A-2869 | Ar.2 | Ar.73 |
| A-2870 | Ar.3 | Ar.73 |
| A-2871 | Ar.4 | Ar.73 |
| A-2872 | Ar.5 | Ar.73 |
| A-2873 | Ar.6 | Ar.73 |
| A-2874 | Ar.7 | Ar.73 |
| A-2875 | Ar.8 | Ar.73 |
| A-2876 | Ar.9 | Ar.73 |
| A-2877 | Ar.10 | Ar.73 |
| A-2878 | Ar.11 | Ar.73 |
| A-2879 | Ar.12 | Ar.73 |
| A-2880 | Ar.13 | Ar.73 |
| A-2881 | Ar.14 | Ar.73 |
| A-2882 | Ar.15 | Ar.73 |
| A-2883 | OCH₃ | Ar.74 |
| A-2884 | SCH₃ | Ar.74 |
| A-2885 | OCF₃ | Ar.74 |
| A-2886 | OCHF₂ | Ar.74 |
| A-2887 | methyl | Ar.74 |
| A-2888 | iso-propyl | Ar.74 |
| A-2889 | ethenyl | Ar.74 |
| A-2890 | ethynyl | Ar.74 |
| A-2891 | Ar.0 | Ar.74 |
| A-2892 | Ar.1 | Ar.74 |
| A-2893 | Ar.2 | Ar.74 |
| A-2894 | Ar.3 | Ar.74 |
| A-2895 | Ar.4 | Ar.74 |
| A-2896 | Ar.5 | Ar.74 |
| A-2897 | Ar.6 | Ar.74 |
| A-2898 | Ar.7 | Ar.74 |
| A-2899 | Ar.8 | Ar.74 |
| A-2900 | Ar.9 | Ar.74 |
| A-2901 | Ar.10 | Ar.74 |
| A-2902 | Ar.11 | Ar.74 |
| A-2903 | Ar.12 | Ar.74 |
| A-2904 | Ar.13 | Ar.74 |
| A-2905 | Ar.14 | Ar.74 |
| A-2906 | Ar.15 | Ar.74 |
| A-2907 | OCH₃ | Ar.75 |
| A-2908 | SCH₃ | Ar.75 |
| A-2909 | OCF₃ | Ar.75 |
| A-2910 | OCHF₂ | Ar.75 |
| A-2911 | methyl | Ar.75 |
| A-2912 | iso-propyl | Ar.75 |
| A-2913 | ethenyl | Ar.75 |
| A-2914 | ethynyl | Ar.75 |
| A-2915 | Ar.0 | Ar.75 |
| A-2916 | Ar.1 | Ar.75 |
| A-2917 | Ar.2 | Ar.75 |
| A-2918 | Ar.3 | Ar.75 |
| A-2919 | Ar.4 | Ar.75 |
| A-2920 | Ar.5 | Ar.75 |
| A-2921 | Ar.6 | Ar.75 |
| A-2922 | Ar.7 | Ar.75 |
| A-2923 | Ar.8 | Ar.75 |
| A-2924 | Ar.9 | Ar.75 |
| A-2925 | Ar.10 | Ar.75 |
| A-2926 | Ar.11 | Ar.75 |
| A-2927 | Ar.12 | Ar.75 |
| A-2928 | Ar.13 | Ar.75 |
| A-2929 | Ar.14 | Ar.75 |
| A-2930 | Ar.15 | Ar.75 |
| A-2931 | OCH₃ | Ar.76 |
| A-2932 | SCH₃ | Ar.76 |
| A-2933 | OCF₃ | Ar.76 |
| A-2934 | OCHF₂ | Ar.76 |
| A-2935 | methyl | Ar.76 |
| A-2936 | iso-propyl | Ar.76 |
| A-2937 | ethenyl | Ar.76 |
| A-2938 | ethynyl | Ar.76 |
| A-2939 | Ar.0 | Ar.76 |
| A-2940 | Ar.1 | Ar.76 |
| A-2941 | Ar.2 | Ar.76 |
| A-2942 | Ar.3 | Ar.76 |
| A-2943 | Ar.4 | Ar.76 |
| A-2944 | Ar.5 | Ar.76 |
| A-2945 | Ar.6 | Ar.76 |
| A-2946 | Ar.7 | Ar.76 |
| A-2947 | Ar.8 | Ar.76 |
| A-2948 | Ar.9 | Ar.76 |
| A-2949 | Ar.10 | Ar.76 |
| A-2950 | Ar.11 | Ar.76 |
| A-2951 | Ar.12 | Ar.76 |
| A-2952 | Ar.13 | Ar.76 |
| A-2953 | Ar.14 | Ar.76 |
| A-2954 | Ar.15 | Ar.76 |
| A-2955 | OCH₃ | Ar.77 |
| A-2956 | SCH₃ | Ar.77 |
| A-2957 | OCF₃ | Ar.77 |
| A-2958 | OCHF₂ | Ar.77 |
| A-2959 | methyl | Ar.77 |
| A-2960 | iso-propyl | Ar.77 |
| A-2961 | ethenyl | Ar.77 |
| A-2962 | ethynyl | Ar.77 |
| A-2963 | Ar.0 | Ar.77 |
| A-2964 | Ar.1 | Ar.77 |
| A-2965 | Ar.2 | Ar.77 |
| A-2966 | Ar.3 | Ar.77 |
| A-2967 | Ar.4 | Ar.77 |
| A-2968 | Ar.5 | Ar.77 |
| A-2969 | Ar.6 | Ar.77 |
| A-2970 | Ar.7 | Ar.77 |
| A-2971 | Ar.8 | Ar.77 |
| A-2972 | Ar.9 | Ar.77 |
| A-2973 | Ar.10 | Ar.77 |
| A-2974 | Ar.11 | Ar.77 |
| A-2975 | Ar.12 | Ar.77 |
| A-2976 | Ar.13 | Ar.77 |
| A-2977 | Ar.14 | Ar.77 |
| A-2978 | Ar.15 | Ar.77 |
| A-2979 | OCH₃ | Ar.78 |
| A-2980 | SCH₃ | Ar.78 |
| A-2981 | OCF₃ | Ar.78 |
| A-2982 | OCHF₂ | Ar.78 |
| A-2983 | methyl | Ar.78 |
| A-2984 | iso-propyl | Ar.78 |
| A-2985 | ethenyl | Ar.78 |
| A-2986 | ethynyl | Ar.78 |
| A-2987 | Ar.0 | Ar.78 |
| A-2988 | Ar.1 | Ar.78 |
| A-2989 | Ar.2 | Ar.78 |
| A-2990 | Ar.3 | Ar.78 |
| A-2991 | Ar.4 | Ar.78 |
| A-2992 | Ar.5 | Ar.78 |
| A-2993 | Ar.6 | Ar.78 |
| A-2994 | Ar.7 | Ar.78 |
| A-2995 | Ar.8 | Ar.78 |
| A-2996 | Ar.9 | Ar.78 |
| A-2997 | Ar.10 | Ar.78 |
| A-2998 | Ar.11 | Ar.78 |
| A-2999 | Ar.12 | Ar.78 |
| A-3000 | Ar.13 | Ar.78 |
| A-3001 | Ar.14 | Ar.78 |
| A-3002 | Ar.15 | Ar.78 |
| A-3003 | OCH₃ | Ar.79 |
| A-3004 | SCH₃ | Ar.79 |
| A-3005 | OCF₃ | Ar.79 |
| A-3006 | OCHF₂ | Ar.79 |
| A-3007 | methyl | Ar.79 |
| A-3008 | iso-propyl | Ar.79 |
| A-3009 | ethenyl | Ar.79 |
| A-3010 | ethynyl | Ar.79 |
| A-3011 | Ar.0 | Ar.79 |
| A-3012 | Ar.1 | Ar.79 |
| A-3013 | Ar.2 | Ar.79 |
| A-3014 | Ar.3 | Ar.79 |
| A-3015 | Ar.4 | Ar.79 |
| A-3016 | Ar.5 | Ar.79 |
| A-3017 | Ar.6 | Ar.79 |
| A-3018 | Ar.7 | Ar.79 |
| A-3019 | Ar.8 | Ar.79 |
| A-3020 | Ar.9 | Ar.79 |
| A-3021 | Ar.10 | Ar.79 |
| A-3022 | Ar.11 | Ar.79 |
| A-3023 | Ar.12 | Ar.79 |
| A-3024 | Ar.13 | Ar.79 |
| A-3025 | Ar.14 | Ar.79 |
| A-3026 | Ar.15 | Ar.79 |
| A-3027 | OCH₃ | Ar.80 |
| A-3028 | SCH₃ | Ar.80 |
| A-3029 | OCF₃ | Ar.80 |
| A-3030 | OCHF₂ | Ar.80 |
| A-3031 | methyl | Ar.80 |
| A-3032 | iso-propyl | Ar.80 |
| A-3033 | ethenyl | Ar.80 |
| A-3034 | ethynyl | Ar.80 |
| A-3035 | Ar.0 | Ar.80 |
| A-3036 | Ar.1 | Ar.80 |
| A-3037 | Ar.2 | Ar.80 |
| A-3038 | Ar.3 | Ar.80 |
| A-3039 | Ar.4 | Ar.80 |
| A-3040 | Ar.5 | Ar.80 |
| A-3041 | Ar.6 | Ar.80 |
| A-3042 | Ar.7 | Ar.80 |
| A-3043 | Ar.8 | Ar.80 |
| A-3044 | Ar.9 | Ar.80 |
| A-3045 | Ar.10 | Ar.80 |
| A-3046 | Ar.11 | Ar.80 |
| A-3047 | Ar.12 | Ar.80 |
| A-3048 | Ar.13 | Ar.80 |
| A-3049 | Ar.14 | Ar.80 |
| A-3050 | Ar.15 | Ar.80 |
| A-3051 | OCH₃ | Ar.81 |
| A-3052 | SCH₃ | Ar.81 |
| A-3053 | OCF₃ | Ar.81 |
| A-3054 | OCHF₂ | Ar.81 |
| A-3055 | methyl | Ar.81 |
| A-3056 | iso-propyl | Ar.81 |
| A-3057 | ethenyl | Ar.81 |
| A-3058 | ethynyl | Ar.81 |
| A-3059 | Ar.0 | Ar.81 |
| A-3060 | Ar.1 | Ar.81 |
| A-3061 | Ar.2 | Ar.81 |
| A-3062 | Ar.3 | Ar.81 |
| A-3063 | Ar.4 | Ar.81 |
| A-3064 | Ar.5 | Ar.81 |
| A-3065 | Ar.6 | Ar.81 |
| A-3066 | Ar.7 | Ar.81 |
| A-3067 | Ar.8 | Ar.81 |
| A-3068 | Ar.9 | Ar.81 |
| A-3069 | Ar.10 | Ar.81 |
| A-3070 | Ar.11 | Ar.81 |
| A-3071 | Ar.12 | Ar.81 |
| A-3072 | Ar.13 | Ar.81 |
| A-3073 | Ar.14 | Ar.81 |
| A-3074 | Ar.15 | Ar.81 |
| A-3075 | OCH₃ | Ar.82 |
| A-3076 | SCH₃ | Ar.82 |
| A-3077 | OCF₃ | Ar.82 |
| A-3078 | OCHF₂ | Ar.82 |
| A-3079 | methyl | Ar.82 |
| A-3080 | iso-propyl | Ar.82 |
| A-3081 | ethenyl | Ar.82 |
| A-3082 | ethynyl | Ar.82 |
| A-3083 | Ar.0 | Ar.82 |
| A-3084 | Ar.1 | Ar.82 |
| A-3085 | Ar.2 | Ar.82 |
| A-3086 | Ar.3 | Ar.82 |
| A-3087 | Ar.4 | Ar.82 |
| A-3088 | Ar.5 | Ar.82 |
| A-3089 | Ar.6 | Ar.82 |
| A-3090 | Ar.7 | Ar.82 |
| A-3091 | Ar.8 | Ar.82 |
| A-3092 | Ar.9 | Ar.82 |
| A-3093 | Ar.10 | Ar.82 |
| A-3094 | Ar.11 | Ar.82 |
| A-3095 | Ar.12 | Ar.82 |
| A-3096 | Ar.13 | Ar.82 |
| A-3097 | Ar.14 | Ar.82 |
| A-3098 | Ar.15 | Ar.82 |
| A-3099 | OCH₃ | Ar.83 |
| A-3100 | SCH₃ | Ar.83 |
| A-3101 | OCF₃ | Ar.83 |
| A-3102 | OCHF₂ | Ar.83 |
| A-3103 | methyl | Ar.83 |
| A-3104 | iso-propyl | Ar.83 |
| A-3105 | ethenyl | Ar.83 |
| A-3106 | ethynyl | Ar.83 |
| A-3107 | Ar.0 | Ar.83 |
| A-3108 | Ar.1 | Ar.83 |
| A-3109 | Ar.2 | Ar.83 |
| A-3110 | Ar.3 | Ar.83 |
| A-3111 | Ar.4 | Ar.83 |
| A-3112 | Ar.5 | Ar.83 |
| A-3113 | Ar.6 | Ar.83 |
| A-3114 | Ar.7 | Ar.83 |
| A-3115 | Ar.8 | Ar.83 |
| A-3116 | Ar.9 | Ar.83 |
| A-3117 | Ar.10 | Ar.83 |
| A-3118 | Ar.11 | Ar.83 |
| A-3119 | Ar.12 | Ar.83 |
| A-3120 | Ar.13 | Ar.83 |
| A-3121 | Ar.14 | Ar.83 |
| A-3122 | Ar.15 | Ar.83 |
| A-3123 | OCH₃ | Ar.84 |
| A-3124 | SCH₃ | Ar.84 |
| A-3125 | OCF₃ | Ar.84 |
| A-3126 | OCHF₂ | Ar.84 |
| A-3127 | methyl | Ar.84 |
| A-3128 | iso-propyl | Ar.84 |
| A-3129 | ethenyl | Ar.84 |
| A-3130 | ethynyl | Ar.84 |
| A-3131 | Ar.0 | Ar.84 |
| A-3132 | Ar.1 | Ar.84 |
| A-3133 | Ar.2 | Ar.84 |
| A-3134 | Ar.3 | Ar.84 |
| A-3135 | Ar.4 | Ar.84 |
| A-3136 | Ar.5 | Ar.84 |
| A-3137 | Ar.6 | Ar.84 |
| A-3138 | Ar.7 | Ar.84 |
| A-3139 | Ar.8 | Ar.84 |
| A-3140 | Ar.9 | Ar.84 |
| A-3141 | Ar.10 | Ar.84 |
| A-3142 | Ar.11 | Ar.84 |
| A-3143 | Ar.12 | Ar.84 |
| A-3144 | Ar.13 | Ar.84 |
| A-3145 | Ar.14 | Ar.84 |
| A-3146 | Ar.15 | Ar.84 |
| A-3147 | OCH₃ | Ar.85 |
| A-3148 | SCH₃ | Ar.85 |
| A-3149 | OCF₃ | Ar.85 |
| A-3150 | OCHF₂ | Ar.85 |
| A-3151 | methyl | Ar.85 |
| A-3152 | iso-propyl | Ar.85 |
| A-3153 | ethenyl | Ar.85 |
| A-3154 | ethynyl | Ar.85 |
| A-3155 | Ar.0 | Ar.85 |
| A-3156 | Ar.1 | Ar.85 |
| A-3157 | Ar.2 | Ar.85 |
| A-3158 | Ar.3 | Ar.85 |
| A-3159 | Ar.4 | Ar.85 |
| A-3160 | Ar.5 | Ar.85 |
| A-3161 | Ar.6 | Ar.85 |
| A-3162 | Ar.7 | Ar.85 |
| A-3163 | Ar.8 | Ar.85 |
| A-3164 | Ar.9 | Ar.85 |
| A-3165 | Ar.10 | Ar.85 |
| A-3166 | Ar.11 | Ar.85 |
| A-3167 | Ar.12 | Ar.85 |
| A-3168 | Ar.13 | Ar.85 |
| A-3169 | Ar.14 | Ar.85 |
| A-3170 | Ar.15 | Ar.85 |
| A-3171 | OCH₃ | Ar.86 |
| A-3172 | SCH₃ | Ar.86 |
| A-3173 | OCF₃ | Ar.86 |
| A-3174 | OCHF₂ | Ar.86 |
| A-3175 | methyl | Ar.86 |
| A-3176 | iso-propyl | Ar.86 |
| A-3177 | ethenyl | Ar.86 |
| A-3178 | ethynyl | Ar.86 |
| A-3179 | Ar.0 | Ar.86 |
| A-3180 | Ar.1 | Ar.86 |
| A-3181 | Ar.2 | Ar.86 |
| A-3182 | Ar.3 | Ar.86 |
| A-3183 | Ar.4 | Ar.86 |
| A-3184 | Ar.5 | Ar.86 |
| A-3185 | Ar.6 | Ar.86 |
| A-3186 | Ar.7 | Ar.86 |
| A-3187 | Ar.8 | Ar.86 |
| A-3188 | Ar.9 | Ar.86 |
| A-3189 | Ar.10 | Ar.86 |
| A-3190 | Ar.11 | Ar.86 |
| A-3191 | Ar.12 | Ar.86 |
| A-3192 | Ar.13 | Ar.86 |
| A-3193 | Ar.14 | Ar.86 |
| A-3194 | Ar.15 | Ar.86 |
| A-3195 | OCH₃ | Ar.87 |
| A-3196 | SCH₃ | Ar.87 |
| A-3197 | OCF₃ | Ar.87 |
| A-3198 | OCHF₂ | Ar.87 |
| A-3199 | methyl | Ar.87 |
| A-3200 | iso-propyl | Ar.87 |
| A-3201 | ethenyl | Ar.87 |
| A-3202 | ethynyl | Ar.87 |
| A-3203 | Ar.0 | Ar.87 |
| A-3204 | Ar.1 | Ar.87 |
| A-3205 | Ar.2 | Ar.87 |
| A-3206 | Ar.3 | Ar.87 |
| A-3207 | Ar.4 | Ar.87 |
| A-3208 | Ar.5 | Ar.87 |
| A-3209 | Ar.6 | Ar.87 |
| A-3210 | Ar.7 | Ar.87 |
| A-3211 | Ar.8 | Ar.87 |
| A-3212 | Ar.9 | Ar.87 |
| A-3213 | Ar.10 | Ar.87 |
| A-3214 | Ar.11 | Ar.87 |
| A-3215 | Ar.12 | Ar.87 |
| A-3216 | Ar.13 | Ar.87 |
| A-3217 | Ar.14 | Ar.87 |
| A-3218 | Ar.15 | Ar.87 |
| A-3219 | OCH₃ | Ar.88 |
| A-3220 | SCH₃ | Ar.88 |
| A-3221 | OCF₃ | Ar.88 |
| A-3222 | OCHF₂ | Ar.88 |
| A-3223 | methyl | Ar.88 |
| A-3224 | iso-propyl | Ar.88 |
| A-3225 | ethenyl | Ar.88 |
| A-3226 | ethynyl | Ar.88 |
| A-3227 | Ar.0 | Ar.88 |
| A-3228 | Ar.1 | Ar.88 |
| A-3229 | Ar.2 | Ar.88 |
| A-3230 | Ar.3 | Ar.88 |
| A-3231 | Ar.4 | Ar.88 |
| A-3232 | Ar.5 | Ar.88 |
| A-3233 | Ar.6 | Ar.88 |
| A-3234 | Ar.7 | Ar.88 |
| A-3235 | Ar.8 | Ar.88 |
| A-3236 | Ar.9 | Ar.88 |
| A-3237 | Ar.10 | Ar.88 |
| A-3238 | Ar.11 | Ar.88 |
| A-3239 | Ar.12 | Ar.88 |
| A-3240 | Ar.13 | Ar.88 |
| A-3241 | Ar.14 | Ar.88 |
| A-3242 | Ar.15 | Ar.88 |
| A-3243 | OCH₃ | Ar.89 |
| A-3244 | SCH₃ | Ar.89 |
| A-3245 | OCF₃ | Ar.89 |
| A-3246 | OCHF₂ | Ar.89 |
| A-3247 | methyl | Ar.89 |
| A-3248 | iso-propyl | Ar.89 |
| A-3249 | ethenyl | Ar.89 |
| A-3250 | ethynyl | Ar.89 |
| A-3251 | Ar.0 | Ar.89 |
| A-3252 | Ar.1 | Ar.89 |
| A-3253 | Ar.2 | Ar.89 |
| A-3254 | Ar.3 | Ar.89 |
| A-3255 | Ar.4 | Ar.89 |
| A-3256 | Ar.5 | Ar.89 |
| A-3257 | Ar.6 | Ar.89 |
| A-3258 | Ar.7 | Ar.89 |
| A-3259 | Ar.8 | Ar.89 |
| A-3260 | Ar.9 | Ar.89 |
| A-3261 | Ar.10 | Ar.89 |
| A-3262 | Ar.11 | Ar.89 |
| A-3263 | Ar.12 | Ar.89 |
| A-3264 | Ar.13 | Ar.89 |
| A-3265 | Ar.14 | Ar.89 |
| A-3266 | Ar.15 | Ar.89 |
| A-3267 | OCH₃ | Ar.90 |
| A-3268 | SCH₃ | Ar.90 |
| A-3269 | OCF₃ | Ar.90 |
| A-3270 | OCHF₂ | Ar.90 |
| A-3271 | methyl | Ar.90 |
| A-3272 | iso-propyl | Ar.90 |
| A-3273 | ethenyl | Ar.90 |
| A-3274 | ethynyl | Ar.90 |
| A-3275 | Ar.0 | Ar.90 |
| A-3276 | Ar.1 | Ar.90 |
| A-3277 | Ar.2 | Ar.90 |
| A-3278 | Ar.3 | Ar.90 |
| A-3279 | Ar.4 | Ar.90 |
| A-3280 | Ar.5 | Ar.90 |
| A-3281 | Ar.6 | Ar.90 |
| A-3282 | Ar.7 | Ar.90 |
| A-3283 | Ar.8 | Ar.90 |
| A-3284 | Ar.9 | Ar.90 |
| A-3285 | Ar.10 | Ar.90 |
| A-3286 | Ar.11 | Ar.90 |
| A-3287 | Ar.12 | Ar.90 |
| A-3288 | Ar.13 | Ar.90 |
| A-3289 | Ar.14 | Ar.90 |
| A-3290 | Ar.15 | Ar.90 |
| A-3291 | Ar.16 | Ar.19 |
| A-3292 | Ar.17 | Ar.19 |
| A-3293 | Ar.18 | Ar.19 |
| A-3294 | Ar.19 | Ar.19 |
| A-3295 | Ar.20 | Ar.19 |
| A-3296 | Ar.21 | Ar.19 |
| A-3297 | Ar.22 | Ar.19 |
| A-3298 | Ar.23 | Ar.19 |
| A-3299 | Ar.24 | Ar.19 |
| A-3300 | Ar.25 | Ar.19 |
| A-3301 | Ar.26 | Ar.19 |
| A-3302 | Ar.27 | Ar.19 |
| A-3303 | Ar.28 | Ar.19 |
| A-3304 | Ar.29 | Ar.19 |
| A-3305 | Ar.30 | Ar.19 |
| A-3306 | Ar.31 | Ar.19 |
| A-3307 | Ar.32 | Ar.19 |
| A-3308 | Ar.33 | Ar.19 |
| A-3309 | Ar.34 | Ar.19 |
| A-3310 | Ar.35 | Ar.19 |
| A-3311 | Ar.36 | Ar.19 |
| A-3312 | Ar.37 | Ar.19 |
| A-3313 | Ar.38 | Ar.19 |
| A-3314 | Ar.39 | Ar.19 |
| A-3315 | Ar.40 | Ar.19 |
| A-3316 | Ar.41 | Ar.19 |
| A-3317 | Ar.42 | Ar.19 |
| A-3318 | Ar.43 | Ar.19 |
| A-3319 | Ar.44 | Ar.19 |
| A-3320 | Ar.45 | Ar.19 |
| A-3321 | Ar.46 | Ar.19 |
| A-3322 | Ar.47 | Ar.19 |
| A-3323 | Ar.48 | Ar.19 |
| A-3324 | Ar.49 | Ar.19 |
| A-3325 | Ar.50 | Ar.19 |
| A-3326 | Ar.51 | Ar.19 |
| A-3327 | Ar.52 | Ar.19 |
| A-3328 | Ar.53 | Ar.19 |
| A-3329 | Ar.54 | Ar.19 |
| A-3330 | Ar.55 | Ar.19 |
| A-3331 | Ar.56 | Ar.19 |
| A-3332 | Ar.57 | Ar.19 |
| A-3333 | Ar.58 | Ar.19 |
| A-3334 | Ar.59 | Ar.19 |
| A-3335 | Ar.60 | Ar.19 |
| A-3336 | Ar.61 | Ar.19 |
| A-3337 | Ar.62 | Ar.19 |
| A-3338 | Ar.63 | Ar.19 |
| A-3339 | Ar.64 | Ar.19 |
| A-3340 | Ar.65 | Ar.19 |
| A-3341 | Ar.66 | Ar.19 |
| A-3342 | Ar.67 | Ar.19 |
| A-3343 | Ar.68 | Ar.19 |
| A-3344 | Ar.69 | Ar.19 |
| A-3345 | Ar.70 | Ar.19 |
| A-3346 | Ar.71 | Ar.19 |
| A-3347 | Ar.72 | Ar.19 |
| A-3348 | Ar.73 | Ar.19 |
| A-3349 | Ar.74 | Ar.19 |
| A-3350 | Ar.75 | Ar.19 |
| A-3351 | Ar.76 | Ar.19 |
| A-3352 | Ar.77 | Ar.19 |
| A-3353 | Ar.78 | Ar.19 |
| A-3354 | Ar.79 | Ar.19 |
| A-3355 | Ar.80 | Ar.19 |
| A-3356 | Ar.81 | Ar.19 |
| A-3357 | Ar.82 | Ar.19 |
| A-3358 | Ar.83 | Ar.19 |
| A-3359 | Ar.84 | Ar.19 |
| A-3360 | Ar.85 | Ar.19 |
| A-3361 | Ar.86 | Ar.19 |
| A-3362 | Ar.87 | Ar.19 |
| A-3363 | Ar.88 | Ar.19 |
| A-3364 | Ar.89 | Ar.19 |
| A-3365 | Ar.90 | Ar.19 |
| A-3366 | Ar.16 | Ar.18 |
| A-3367 | Ar.17 | Ar.18 |
| A-3368 | Ar.18 | Ar.18 |
| A-3369 | Ar.19 | Ar.18 |
| A-3370 | Ar.20 | Ar.18 |
| A-3371 | Ar.21 | Ar.18 |
| A-3372 | Ar.22 | Ar.18 |
| A-3373 | Ar.23 | Ar.18 |
| A-3374 | Ar.24 | Ar.18 |
| A-3375 | Ar.25 | Ar.18 |
| A-3376 | Ar.26 | Ar.18 |
| A-3377 | Ar.27 | Ar.18 |
| A-3378 | Ar.28 | Ar.18 |
| A-3379 | Ar.29 | Ar.18 |
| A-3380 | Ar.30 | Ar.18 |
| A-3381 | Ar.31 | Ar.18 |
| A-3382 | Ar.32 | Ar.18 |
| A-3383 | Ar.33 | Ar.18 |
| A-3384 | Ar.34 | Ar.18 |
| A-3385 | Ar.35 | Ar.18 |
| A-3386 | Ar.36 | Ar.18 |
| A-3387 | Ar.37 | Ar.18 |
| A-3388 | Ar.38 | Ar.18 |
| A-3389 | Ar.39 | Ar.18 |
| A-3390 | Ar.40 | Ar.18 |
| A-3391 | Ar.41 | Ar.18 |
| A-3392 | Ar.42 | Ar.18 |
| A-3393 | Ar.43 | Ar.18 |
| A-3394 | Ar.44 | Ar.18 |
| A-3395 | Ar.45 | Ar.18 |
| A-3396 | Ar.46 | Ar.18 |
| A-3397 | Ar.47 | Ar.18 |
| A-3398 | Ar.48 | Ar.18 |
| A-3399 | Ar.49 | Ar.18 |
| A-3400 | Ar.50 | Ar.18 |
| A-3401 | Ar.51 | Ar.18 |
| A-3402 | Ar.52 | Ar.18 |
| A-3403 | Ar.53 | Ar.18 |
| A-3404 | Ar.54 | Ar.18 |
| A-3405 | Ar.55 | Ar.18 |
| A-3406 | Ar.56 | Ar.18 |
| A-3407 | Ar.57 | Ar.18 |
| A-3408 | Ar.58 | Ar.18 |
| A-3409 | Ar.59 | Ar.18 |
| A-3410 | Ar.60 | Ar.18 |
| A-3411 | Ar.61 | Ar.18 |
| A-3412 | Ar.62 | Ar.18 |
| A-3413 | Ar.63 | Ar.18 |
| A-3414 | Ar.64 | Ar.18 |
| A-3415 | Ar.65 | Ar.18 |
| A-3416 | Ar.66 | Ar.18 |
| A-3417 | Ar.67 | Ar.18 |
| A-3418 | Ar.68 | Ar.18 |
| A-3419 | Ar.69 | Ar.18 |
| A-3420 | Ar.70 | Ar.18 |
| A-3421 | Ar.71 | Ar.18 |
| A-3422 | Ar.72 | Ar.18 |
| A-3423 | Ar.73 | Ar.18 |
| A-3424 | Ar.74 | Ar.18 |
| A-3425 | Ar.75 | Ar.18 |
| A-3426 | Ar.76 | Ar.18 |
| A-3427 | Ar.77 | Ar.18 |
| A-3428 | Ar.78 | Ar.18 |
| A-3429 | Ar.79 | Ar.18 |
| A-3430 | Ar.80 | Ar.18 |
| A-3431 | Ar.81 | Ar.18 |
| A-3432 | Ar.82 | Ar.18 |
| A-3433 | Ar.83 | Ar.18 |
| A-3434 | Ar.84 | Ar.18 |
| A-3435 | Ar.85 | Ar.18 |
| A-3436 | Ar.86 | Ar.18 |
| A-3437 | Ar.87 | Ar.18 |
| A-3438 | Ar.88 | Ar.18 |
| A-3439 | Ar.89 | Ar.18 |
| A-3440 | Ar.90 | Ar.18 |
| A-3441 | Ar.81 | Ar.0 |
| A-3442 | Ar.81 | Ar.1 |
| A-3443 | Ar.81 | Ar.2 |
| A-3444 | Ar.81 | Ar.3 |
| A-3445 | Ar.81 | Ar.4 |
| A-3446 | Ar.81 | Ar.5 |
| A-3447 | Ar.81 | Ar.6 |
| A-3448 | Ar.81 | Ar.7 |
| A-3449 | Ar.81 | Ar.8 |
| A-3450 | Ar.81 | Ar.9 |
| A-3451 | Ar.81 | Ar.10 |
| A-3452 | Ar.81 | Ar.11 |
| A-3453 | Ar.81 | Ar.12 |
| A-3454 | Ar.81 | Ar.13 |
| A-3455 | Ar.81 | Ar.14 |
| A-3456 | Ar.81 | Ar.15 |
| A-3457 | Ar.82 | Ar.0 |
| A-3458 | Ar.82 | Ar.1 |
| A-3459 | Ar.82 | Ar.2 |
| A-3460 | Ar.82 | Ar.3 |
| A-3461 | Ar.82 | Ar.4 |
| A-3462 | Ar.82 | Ar.5 |
| A-3463 | Ar.82 | Ar.6 |
| A-3464 | Ar.82 | Ar.7 |
| A-3465 | Ar.82 | Ar.8 |
| A-3466 | Ar.82 | Ar.9 |
| A-3467 | Ar.82 | Ar.10 |
| A-3468 | Ar.82 | Ar.11 |
| A-3469 | Ar.82 | Ar.12 |
| A-3470 | Ar.82 | Ar.13 |
| A-3471 | Ar.82 | Ar.14 |
| A-3472 | Ar.82 | Ar.15 |
| A-3473 | Ar.83 | Ar.0 |
| A-3474 | Ar.83 | Ar.1 |
| A-3475 | Ar.83 | Ar.2 |
| A-3476 | Ar.83 | Ar.3 |
| A-3477 | Ar.83 | Ar.4 |
| A-3478 | Ar.83 | Ar.5 |
| A-3479 | Ar.83 | Ar.6 |
| A-3480 | Ar.83 | Ar.7 |
| A-3481 | Ar.83 | Ar.8 |
| A-3482 | Ar.83 | Ar.9 |
| A-3483 | Ar.83 | Ar.10 |
| A-3484 | Ar.83 | Ar.11 |
| A-3485 | Ar.83 | Ar.12 |
| A-3486 | Ar.83 | Ar.13 |
| A-3487 | Ar.83 | Ar.14 |
| A-3488 | Ar.83 | Ar.15 |
| A-3489 | Ar.84 | Ar.0 |
| A-3490 | Ar.84 | Ar.1 |
| A-3491 | Ar.84 | Ar.2 |
| A-3492 | Ar.84 | Ar.3 |
| A-3493 | Ar.84 | Ar.4 |
| A-3494 | Ar.84 | Ar.5 |
| A-3495 | Ar.84 | Ar.6 |
| A-3496 | Ar.84 | Ar.7 |
| A-3497 | Ar.84 | Ar.8 |
| A-3498 | Ar.84 | Ar.9 |
| A-3499 | Ar.84 | Ar.10 |
| A-3500 | Ar.84 | Ar.11 |
| A-3501 | Ar.84 | Ar.12 |
| A-3502 | Ar.84 | Ar.13 |
| A-3503 | Ar.84 | Ar.14 |
| A-3504 | Ar.84 | Ar.15 |
| A-3505 | Ar.85 | Ar.0 |
| A-3506 | Ar.85 | Ar.1 |
| A-3507 | Ar.85 | Ar.2 |
| A-3508 | Ar.85 | Ar.3 |
| A-3509 | Ar.85 | Ar.4 |
| A-3510 | Ar.85 | Ar.5 |
| A-3511 | Ar.85 | Ar.6 |
| A-3512 | Ar.85 | Ar.7 |
| A-3513 | Ar.85 | Ar.8 |
| A-3514 | Ar.85 | Ar.9 |
| A-3515 | Ar.85 | Ar.10 |
| A-3516 | Ar.85 | Ar.11 |
| A-3517 | Ar.85 | Ar.12 |
| A-3518 | Ar.85 | Ar.13 |
| A-3519 | Ar.85 | Ar.14 |
| A-3520 | Ar.85 | Ar.15 |
| A-3521 | Ar.86 | Ar.0 |
| A-3522 | Ar.86 | Ar.1 |
| A-3523 | Ar.86 | Ar.2 |
| A-3524 | Ar.86 | Ar.3 |
| A-3525 | Ar.86 | Ar.4 |
| A-3526 | Ar.86 | Ar.5 |
| A-3527 | Ar.86 | Ar.6 |
| A-3528 | Ar.86 | Ar.7 |
| A-3529 | Ar.86 | Ar.8 |
| A-3530 | Ar.86 | Ar.9 |
| A-3531 | Ar.86 | Ar.10 |
| A-3532 | Ar.86 | Ar.11 |
| A-3533 | Ar.86 | Ar.12 |
| A-3534 | Ar.86 | Ar.13 |
| A-3535 | Ar.86 | Ar.14 |
| A-3536 | Ar.86 | Ar.15 |
| A-3537 | Ar.87 | Ar.0 |
| A-3538 | Ar.87 | Ar.1 |
| A-3539 | Ar.87 | Ar.2 |
| A-3540 | Ar.87 | Ar.3 |
| A-3541 | Ar.87 | Ar.4 |
| A-3542 | Ar.87 | Ar.5 |
| A-3543 | Ar.87 | Ar.6 |
| A-3544 | Ar.87 | Ar.7 |
| A-3545 | Ar.87 | Ar.8 |
| A-3546 | Ar.87 | Ar.9 |
| A-3547 | Ar.87 | Ar.10 |
| A-3548 | Ar.87 | Ar.11 |
| A-3549 | Ar.87 | Ar.12 |
| A-3550 | Ar.87 | Ar.13 |
| A-3551 | Ar.87 | Ar.14 |
| A-3552 | Ar.87 | Ar.15 |
| A-3553 | Ar.88 | Ar.0 |
| A-3554 | Ar.88 | Ar.1 |
| A-3555 | Ar.88 | Ar.2 |
| A-3556 | Ar.88 | Ar.3 |
| A-3557 | Ar.88 | Ar.4 |
| A-3558 | Ar.88 | Ar.5 |
| A-3559 | Ar.88 | Ar.6 |
| A-3560 | Ar.88 | Ar.7 |
| A-3561 | Ar.88 | Ar.8 |
| A-3562 | Ar.88 | Ar.9 |
| A-3563 | Ar.88 | Ar.10 |
| A-3564 | Ar.88 | Ar.11 |
| A-3565 | Ar.88 | Ar.12 |
| A-3566 | Ar.88 | Ar.13 |
| A-3567 | Ar.88 | Ar.14 |
| A-3568 | Ar.88 | Ar.15 |
| A-3569 | Ar.89 | Ar.0 |
| A-3570 | Ar.89 | Ar.1 |
| A-3571 | Ar.89 | Ar.2 |
| A-3572 | Ar.89 | Ar.3 |
| A-3573 | Ar.89 | Ar.4 |
| A-3574 | Ar.89 | Ar.5 |
| A-3575 | Ar.89 | Ar.6 |
| A-3576 | Ar.89 | Ar.7 |
| A-3577 | Ar.89 | Ar.8 |
| A-3578 | Ar.89 | Ar.9 |
| A-3579 | Ar.89 | Ar.10 |
| A-3580 | Ar.89 | Ar.11 |
| A-3581 | Ar.89 | Ar.12 |
| A-3582 | Ar.89 | Ar.13 |
| A-3583 | Ar.89 | Ar.14 |
| A-3584 | Ar.89 | Ar.15 |
| A-3585 | Ar.90 | Ar.0 |
| A-3586 | Ar.90 | Ar.1 |
| A-3587 | Ar.90 | Ar.2 |
| A-3588 | Ar.90 | Ar.3 |
| A-3589 | Ar.90 | Ar.4 |
| A-3590 | Ar.90 | Ar.5 |
| A-3591 | Ar.90 | Ar.6 |
| A-3592 | Ar.90 | Ar.7 |
| A-3593 | Ar.90 | Ar.8 |
| A-3594 | Ar.90 | Ar.9 |
| A-3595 | Ar.90 | Ar.10 |
| A-3596 | Ar.90 | Ar.11 |
| A-3597 | Ar.90 | Ar.12 |
| A-3598 | Ar.90 | Ar.13 |
| A-3599 | Ar.90 | Ar.14 |
| A-3600 | Ar.90 | Ar.15 |

Amongst compounds of the formulae Ia or Ib, preference is also given to the compounds defined in the following tables 2 to 24:

Table 2: Compounds of the formulae Ia or Ib and their mixtures, wherein X is S, A is CH₂, R^{2a} or R^{2b} is hydrogen, R^{3a}, R^{3b}, R^{3c} and R^{3d} are hydrogen and wherein R¹ and Ar have the meanings given in each line of table B. These compounds are also referred to as Ia-B-1 to Ia-B-3600 or Ib-B-1 to Ib-B-3600.

Table 3: Compounds of the formulae Ia or Ib and their mixtures, wherein X is NH, A is CH₂, R^{2a} or R^{2b} is hydrogen, R^{3a}, R^{3b}, R^{3c} and R^{3d} are hydrogen and wherein R¹ and Ar have the meanings given in each line of table B. These compounds are also referred to as Ia-C-1 to Ia-C-3600 or Ib-C-1 to Ib-C-3600.

Table 4: Compounds of the formulae Ia or Ib and their mixtures, wherein X is NCH₃, A is CH₂, R^{2a} or R^{2b} is hydrogen, R^{3a}, R^{3b}, R^{3c} and R^{3d} are hydrogen and wherein R¹ and Ar have the meanings given in each line of table B. These compounds are also referred to as Ia-D-1 to Ia-D-3600 or Ib-D-1 to Ib-D-3600.

Table 5: Compounds of the formulae Ia or Ib and their mixtures, wherein X is O, A is CH₂, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-E-1 to Ia-E-3600 or Ib-E-1 to Ib-E-3600.

Table 6: Compounds of the formulae Ia or Ib and their mixtures, wherein X is S, A is CH₂, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-F-1 to Ia-F-3600 or Ib-F-1 to Ib-F-3600.

Table 7: Compounds of the formulae Ia or Ib and their mixtures, wherein X is NH, A is CH₂, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-G-1 to Ia-G-3600 or Ib-G-1 to Ib-G-3600.

Table 8: Compounds of the formulae Ia or Ib and their mixtures, wherein X is NCH₃, A is CH₂, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-H-1 to Ia-H-3600 or Ib-H-1 to Ib-H-3600.

Table 9: Compounds of the formulae Ia or Ib and their mixtures, wherein X is O, A is CH₂, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-J-1 to Ia-J-3600 or Ib-J-1 to Ib-J-3600.

Table 10: Compounds of the formulae Ia or Ib and their mixtures, wherein X is S, A is CH₂, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-K-1 to Ia-K-3600 or Ib-K-1 to Ib-K-3600.

Table 11: Compounds of the formulae Ia or Ib and their mixtures, wherein X is NH, A is CH₂, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-L-1 to Ia-L-3600 or Ib-L-1 to Ib-L-3600.

Table 12: Compounds of the formulae Ia or Ib and their mixtures, wherein X is NCH₃,

A is CH₂, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-M-1 to Ia-M-3600 or Ib-M-1 to Ib-M-3600.

Table 13: Compounds of the formulae Ia or Ib and their mixtures, wherein X is O, A is CHCH₃, R^{2a} or R^{2b} is H, R^{3a}, R^{3b}, R^{3c} and R^{3d} are hydrogen and wherein R¹ and Ar have the meanings given in each line of table B. These compounds are also referred to as Ia-N-1 to Ia-N-3600 or Ib-N-1 to Ib-N-3600.

Table 14: Compounds of the formulae Ia or Ib and their mixtures, wherein X is S, A is CHCH₃, R^{2a} or R^{2b} is H, R^{3a}, R^{3b}, R^{3c} and R^{3d} are hydrogen and wherein R¹ and Ar have the meanings given in each line of table B. These compounds are also referred to as Ia-O-1 to Ia-O-3600 or Ib-O-1 to Ib-O-3600.

Table 15: Compounds of the formulae Ia or Ib and their mixtures, wherein X is NH, A is CHCH₃, R^{2a} or R^{2b} is H, R^{3a}, R^{3b}, R^{3c} and R^{3d} are hydrogen and wherein R¹ and Ar have the meanings given in each line of table B. These compounds are also referred to as Ia-P-1 to Ia-P-3600 or Ib-P-1 to Ib-P-3600.

Table 16: Compounds of the formulae Ia or Ib and their mixtures, wherein X is NCH₃,

A is CHCH₃, R^{2a} or R^{2b} is H, R^{3a}, R^{3b}, R^{3c} and R^{3d} are hydrogen and wherein R¹ and Ar have the meanings given in each line of table B. These compounds are also referred to as Ia-Q-1 to Ia-Q-3600 or Ib-Q-1 to Ib-Q-3600.

Table 17: Compounds of the formulae Ia or Ib and their mixtures, wherein X is O, A is CHCH₃, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-R-1 to Ia-R-3600 or Ib-R-1 to Ib-R-3600.

Table 18: Compounds of the formulae Ia or Ib and their mixtures, wherein X is S, A is CHCH₃, R^{2a} or R^{2b} is CH₃,R^{3a}, R^{3b}, R^{3c} and R^{3d} are hydrogen and wherein R¹ and Ar have the meanings given in each line of table B. These compounds are also referred to as Ia-S-1 to Ia-S-3600 or Ib-S-1 to Ib-S-3600.

Table 19: Compounds of the formulae Ia or Ib and their mixtures, wherein X is NH, A is CHCH₃, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-T-1 to Ia-T-3600 or Ib-T-1 to Ib-T-3600.

Table 20: Compounds of the formulae Ia or Ib and their mixtures, wherein X is NCH₃, A is CHCH₃, R^{2a} or R^{2b} is CH₃, R^{3a}, R^{3b}, R^{3c} and R^{3d} are hydrogen and wherein R¹ and Ar have the meanings given in each line of table B. These compounds are also referred to as Ia-U-1 to Ia-U-3600 or Ib-U-1 to Ib-U-3600.

Table 21: Compounds of the formulae Ia or Ib and their mixtures, wherein X is O, A is CHCH₃, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-V-1 to Ia-V-3600 or Ib-V-1 to Ib-V-3600.

Table 22: Compounds of the formulae Ia or Ib and their mixtures, wherein X is S, A is CHCH₃, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-W-1 to Ia-W-3600 or Ib-W-1 to Ib-W-3600.

Table 23: Compounds of the formulae Ia or Ib and their mixtures, wherein X is NH, A is CHCH₃, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-X-1 to Ia-X-3600 or Ib-X-1 to Ib-X-3600.

Table 24: Compounds of the formulae Ia or Ib and their mixtures, wherein X is NCH₃, A is CHCH₃, R^{2a} or R^{2b} is CN, R^{3a}, R^{3b}, R^{3c} and R^{3d} are hydrogen and wherein R¹ and Ar have the meanings given in each line of table B. These compounds are also referred to as Ia-Y-1 to Ia-Y-3600 or Ib-Y-1 to Ib-Y-3600.

Table 25: Compounds of the formulae Ia or Ib and their mixtures, wherein X is S, A is CH₂, R^{2a} or R^{2b} is C(O)CH₃, R^{3a}, R^{3b}, R^{3c} and R^{3d} are hydrogen and wherein R¹ and Ar have the meanings given in each line of table B. These compounds are also referred to as Ia-Z-1 to Ia-Z-3600 or Ib-Z-1 to Ib-Z-3600.

Table 26: Compounds of the formulae Ia or Ib and their mixtures, wherein X is S, A is CH₂, R^{2a} or R^{2b} is C(O)CH₂OCH₃, R^{3a}, R^{3b}, R^{3c} and R^{3d} are hydrogen and wherein R¹ and Ar have the meanings given in each line of table B. These compounds are also referred to as Ia-AA-1 to Ia-AA-3600 or Ib-AA-1 to Ib-AA-3600.

Table 27: Compounds of the formulae Ia or Ib and their mixtures, wherein X is S, A is CH₂, R^{2a} or R^{2b} is C(O)—O—C(CH₃)₃, R^{3a}, R^{3b}, R^{3c} and R^{3d} are hydrogen and wherein R¹ and Ar have the meanings given in each line of table B. These compounds are also referred to as Ia-AB-1 to Ia-AB-3600 or Ib-AB-1 to Ib-AB-3600.

Table 28: Compounds of the formulae Ia or Ib and their mixtures, wherein X is S, A is CH₂, R^{2a} or R^{2b} is CH₂—Ar.0, R^{3a}, R^{3b}, R^{3c} and R^{3d} are hydrogen and wherein R¹ and Ar have the meanings given in each line of table B. These compounds are also referred to as Ia-AC-1 to Ia-AC-3600 or Ib-AC-1 to Ib-AC-3600.

Table 29: Compounds of the formulae Ia or Ib and their mixtures, wherein X is S, A is CH₂, R^{2a} or R^{2b} is CH₂—Ar.1, R^{3a}, R^{3b}, R^{3c} and R^{3d} are hydrogen and wherein R¹ and Ar have the meanings given in each line of table B. These compounds are also referred to as Ia-AD-1 to Ia-AD-3600 or Ib-AD-1 to Ib-AD-3600.

Table 30: Compounds of the formulae Ia or Ib and their mixtures, wherein X is S, A is CH₂, R^{2a} or R^{2b} is CH₂—Ar.6, R^{3a}, R^{3b}, R^{3c} and R^{3d} are hydrogen and wherein R¹ and Ar have the meanings given in each line of table B. These compounds are also referred to as Ia-AE-1 to Ia-AE-3600 or Ib-AE-1 to Ib-AE-3600.

Table 31: Compounds of the formulae Ia or Ib and their mixtures, wherein X is S, A is CH₂, R^{2a} or R^{2b} is CH₂—Ar.14, R^{3a}, R^{3b}, R^{3c} and R^{3d} are hydrogen and wherein R¹ and Ar have the meanings given in each line of table B. These compounds are also referred to as Ia-AF-1 to Ia-AF-3600 or Ib-AF-1 to Ib-AF-3600.

Table 32: Compounds of the formulae Ia or Ib and their mixtures, wherein X is S, A is CH₂, R^{2a} or R^{2b} is CH₂—Ar.15, R^{3a}, R^{3b}, R^{3c} and R^{3d} are hydrogen and wherein R¹ and Ar have the meanings given in each line of table B. These compounds are also referred to as Ia-AG-1 to Ia-AG-3600 Ib-AG-1 to Ib-AG-3600.

Table 33: Compounds of the formulae Ia or Ib and their mixtures, wherein X is S, A is CH₂, R^{2a} or R^{2b} is CH₂—Ar.16, R^{3a}, R^{3b}, R^{3c} and R^{3d} are hydrogen and wherein R¹ and Ar have the meanings given in each line of table B. These compounds are also referred to as Ia-AH-1 to Ia-AH-3600 Ib-AH-1 to Ib-AH-3600.

Table 34: Compounds of the formulae Ia or Ib and their mixtures, wherein X is S, A is CH₂, R^{2a} or R^{2b} is CH₂—Ar.19, R^{3a}, R^{3b}, R^{3c} and R^{3d} are hydrogen and wherein R¹ and Ar have the meanings given in each line of table B. These compounds are also referred to as Ia-AJ-1 to Ia-AJ-3600 Ib-AJ-1 to Ib-AJ-3600.

Table 35: Compounds of the formulae Ia or Ib and their mixtures, wherein X is S, A is CH₂, R^{2a} or R^{2b} is CH₂—Ar.79, R^{3a}, R^{3b}, R^{3c} and R^{3d} are hydrogen and wherein R¹ and Ar have the meanings given in each line of table B. These compounds are also referred to as Ia-AK-1 to Ia-AK-3600 Ib-AK-1 to Ib-AK-3600.

Table 36: Compounds of the formulae Ia or Ib and their mixtures, wherein X is S, A is CH₂, R^{2a} or R^{2b} is CH₂—Ar.80, R^{3a}, R^{3b}, R^{3c} and R^{3d} are hydrogen and wherein R¹ and Ar have the meanings given in each line of table B. These compounds are also referred to as Ia-AK-1 to Ia-AK-3600 Ib-AK-1 to Ib-AK-3600.

The compounds of the present invention can be e.g. prepared from the corresponding monosubstituted hydrazines 11 by the synthetic routes outlined in schemes 1, 2, 2a, 3 and 4. In the following schemes the variables A, Ar, X, R¹, R^{3a}, R^{3b}, R^{3c} and R^{3d} are as defined above, if not stated otherwise.

Compounds of the formula Ia, with X being S or O and R^{2a} being H can be obtained according to the methods outlined in schemes 1, 2 and 3:

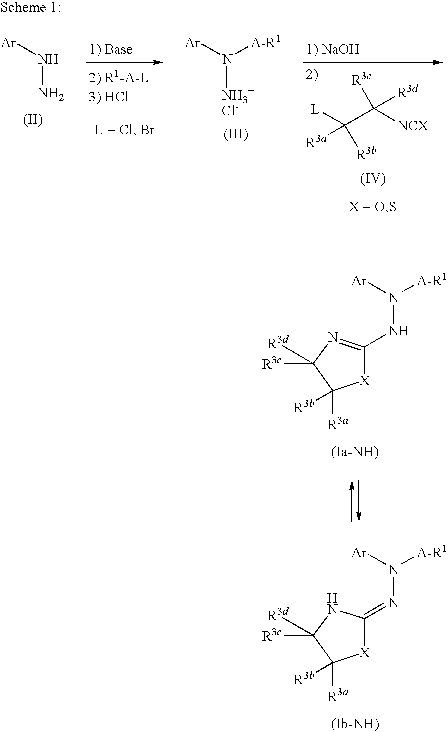

According to the method outlined in scheme 1, a monosubstituted hydrazine compound II is reacted with a compound R¹A-L posessing a leaving group L, which is suitable to undergo a substitution reaction under basic conditions such as chlorine or bromine or arylsulfonate such as tosylate or alkylsulfonate such as mesylate. The thus formed N,N-disubstituted hydrazine compound III can conventionally be isolated as its hydrochloride (see e.g. U. Lerch, J. König, Synthesis, 157-158 (1983) or M. De Angelis, F. Stossi, K. A. Carlson, B. S. Katzenellenbogen, J. A. Katzenellenbogen, J. Med. Chem., 48, 1132-1144 (2005)). In the final step an isocyanate (X═O) or a thioisocyanate (X═S) of the general formula IV is reacted with the salt of the N,N-disubstituted hydrazine compound (III) under basic conditions to form the (azolin-2-yl)hydrazino compound Ia with R^{2a}═H and their tautomeric (3H-azolin-2-yliden)hydrazino compounds Ib with R²b═H. This reaction scheme can easily be adopted using standard techniques of organic chemistry which are well known by a skilled person in the field of organic synthesis.

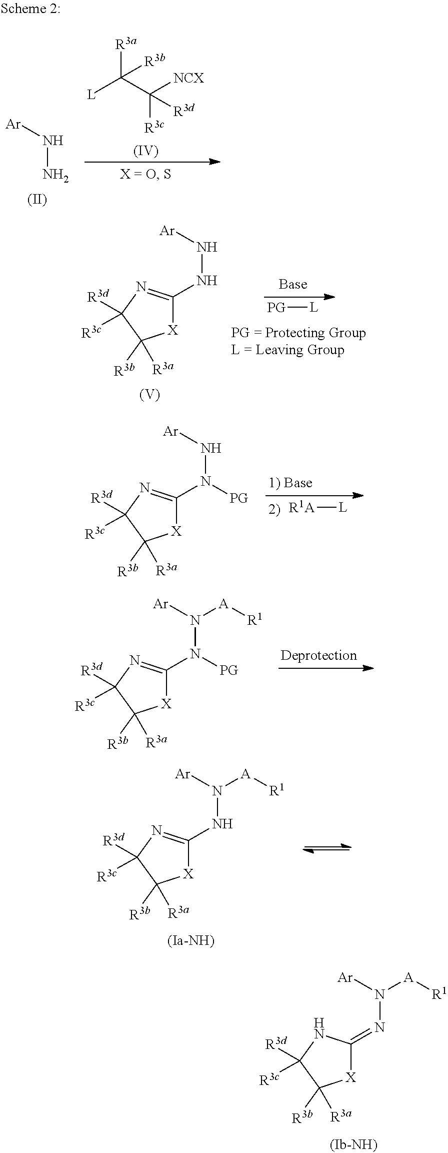

According to the method outlined in scheme 2, a monosubstituted hydrazine compound II can directly be reacted with an isocyanate or a thioisocyanate IV, which possesses a suitable leaving group for subsequent cyclization. Suitable leaving groups are in particular halogen such as chlorine or bromine, arylsulfonate such as tosylate or alkylsulfonate such as mesylate. Thereby, an (azolin-2-yl)hydrazino compound V is obtained which, upon use of usual protecting group chemistry and N-alkylation as mentioned above, yields the (azolin-2-yl)hydrazino compound Ia with R^{2a}═H and its tautomer Ib. Suitable protecting groups are those usually used for amines and well known in the art, e.g. C(O)CF₃, C(O)OMe, alkoxycarbonyls like Boc, benzyloxycarbonyls like Z or Cbz (see also P. J. Kocienski, protecting groups, corrected edition, ed. D. Enders, R. Noyori, B. M. Trost, Thieme (2000), p. 185-238).

Compounds of the formula Ia, with X being N and R^{2a} being H can be obtained according to the methods outlined in schemes 3 and 4, the tautomeric compounds Ib are present in equilibrium:

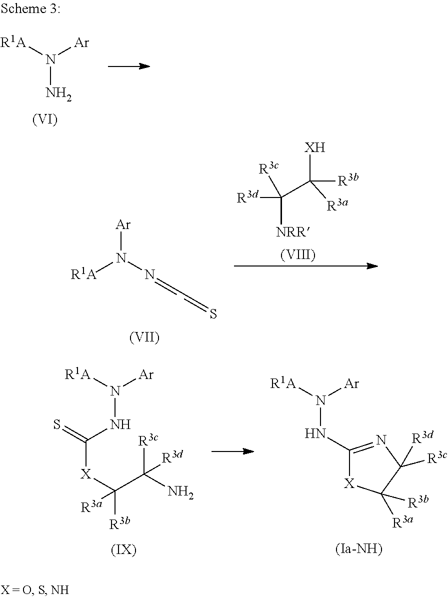

In scheme 3, the variables R and R′ in formula VIII independently of each other represent hydrogen or a protective group or NRR′ is a phthalimidoyl radical. According to the method outlined in Scheme 3, a N,N-disubstituted hydrazine VI can be converted into the corresponding isothiocyanate VII by conventional means, e.g. by reacting VI with thiophosgene (see e.g. Houben-Weyl, E4, “Methoden der Organischen Chemie”, chapter IIIc, pp. 837-842, Georg Thieme Verlag 1983).

The compound VII is then reacted with a 1,2 diaminoethane, a 2-aminoethanol or a 2-aminothioethanol of the formula VIII, thereby obtaining the thiourea derivative of the formula IX. If R and R′ in formula A are both hydrogen, compound VIII is preferably used as its hydrohalide, in particular as the hydrochloride to avoid side reactions. The reaction of VII and VIII can be performed by analogy to standard methods of organic chemistry (see e.g. Tetrahedron 60, 9883-9888 (2004) or Biotech. Biochem., 56(7), 1062-1065 (1992) or J. Org. Chem. 1963, 28, 3140-3144).

The thus obtained thiourea derivatives IX can be cyclized by conventional means, thereby obtaining the desired compound of the formula Ia-NH, wherein R^{2b} and R⁴ are hydrogen. Cyclization of compound IX can be achieved e.g. via intermediate carbodiimide formation and amine addition with e.g. Tosylchloride/NaOH (see, for example Tetrahedron 60, 9883-9888 (2004)) or yellow mercury (II) according to Synthesis, 482-484 (1982).

Some N,N-disubstituted hydrazines VI are commercially available and their preparation is known from the literature or said compounds can be prepared by conventional methods.

Compounds of the formula Ia-C and their tautomers Ib-C, can also be obtained by the method outlined in Scheme 4.

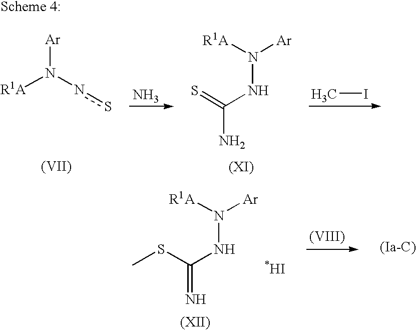

The isothiocyanate VII is converted to the corresponding thiourea XI, which subsequently is treated with methyl iodide to yield the isothiuronium salt XII. Finally, the intermediate XII is reacted with an 1,2-diaminoethane VII (see for example U.S. Pat. No. 2,899,426).

In order to obtain compounds of the formulae Ia and Ib, wherein R^{2a} or R^{2b} is C₁-C₆-alkyloxycarbonyl or C₁-C₆-alkylthiocarbonyl, the starting material is reacted with a suitable haloformiate of the formula R^{b}—C(O)—Hal, wherein Hal is halogen, especially chlorine and wherein R^{b} is C₁-C₆-alkyloxy or C₁-C₆-alkylthio. The reaction can be performed by routine methods described in standard textbooks on organic synthesis, see e.g. J. March, Advanced Organic Synthesis, 3rd ed. John Wiley and Sons.

A cyano group can be introduced as a radical R^{2a} or R^{2b} e.g. by reaction of the starting material with bromocyan according to the methods described in the experimental part of the present application. The introduction of nitro groups as radicals R^{2a} or R^{2b} can be performed by reacting compounds Ia or Ib with R^{2a} or R^{2b} being H with nitronium source according to standard methods well known in the art.

The group (SO₂)NR^{a}R^{b} can be introduced as a radical R^{2a} or R^{2b} e.g. by reacting the starting material with the chlorosulfonamide Cl—(SO₂)NR^{a}R^{b} according to routine methods described in standard textbooks on organic synthesis, see e.g. J. March, Advanced Organic Synthesis, 3rd ed. John Wiley and Sons.

The group C(O)NR^{a}R^{b} can be introduced as a radical R^{2a} or R^{2b} e.g. by reacting the starting material with the chloroformamide Cl—C(O)—NR^{a}R^{b} or by reaction with isocyanates OCN-R^{a} for R^{b} being hydrogen.

The particular reaction mixtures are worked up, as a rule, by conventional methods, for example by removing the solvent, distributing the residue in a mixture of water and a suitable organic solvent and isolating the product from the organic phase.

The (azolin-2-yl)hydrazino compounds (Ia) and their related (3H-azolin-2-yliden)hydrazino compounds (Ib) may be obtained in the preparation as isomer mixtures, which however can, if desired, be separated into the pure isomers by conventional methods, for example by crystallization or chromatography (if necessary, over an optically active adsorbate). Pure optically active isomers can be synthesized, for example, from corresponding optically active starting materials.

As a rule, (azolin-2-yl)hydrazino compounds (Ia) and their related (3H-azolin-2-yliden)hydrazino compounds (Ib) can be prepared by the methods described above. However, in individual cases, certain compounds Ia or Ib can also advantageously be prepared from other compounds Ia or Ib by ester hydrolysis, amidation, esterification, ether cleavage, olefination, reduction, oxidation, cross-coupling reactions or cyclization reactions at the positions of the radical R^{y1} or R^{y2} or by ester hydrolysis, transesterification, ether cleavage or oxidation at the positions of the radical R^{2a}, R^{2b} or R⁴.

Due to their excellent activity, the compounds of the general formulae Ia and Ib may be used for controlling animal pests, selected harmful insects, acarids and nematodes. Accordingly, the invention further provides agriculturally composition for combating such animal pests, which comprises such an amount of at least one compound of the general formulae Ia and/or Ib or at least an agriculturally useful salt of Ia and/or Ib and at least one inert liquid and/or solid agronomically acceptable carrier that it has a pesticidal action and, if desired, at least one surfactant.

Such a composition may contain a single active compound of the general formulae Ia or Ib or a mixture of several active compounds Ia and/or Ib according to the present invention. The composition according to the present invention may comprise an individual isomer or mixtures of isomers as well as individual tautomers or mixtures of tautomers.

The compounds of the formulae Ia and Ib as well as the salts thereof and the pesticidal compositions comprising them are in particular suitable for efficiently controlling arthropodal pests such as arachnids and insects as well as nematodes.

In particular, they are suitable for controlling insect pests, such as insects from the order of the

lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,
beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa
thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,
fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Scutigera coleoptrata,
milipedes (Diplopoda), e.g. Narceus spp.,
earwigs (Dermaptera), e.g. forficula auricularia, and
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

The compounds of the formulae Ia and Ib and their salts are also useful for controlling nematodes, especially plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the formulae Ia and Ib and their salts are also useful for controlling Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa,

Compounds of the formulae Ia and Ib and their salts are particularly useful for controlling piercing sucking insects, in particular those of the order hemiptera

For use in a method according to the present invention, the compounds Ia or Ib or their salts can be converted into the customary formulations, e.g. solutions, emulsions, suspensions, dusts, powders, pastes and granules. The use form depends on the particular purpose; it is intended to ensure in each case a fine and uniform distribution of the compound according to the invention.

The formulations are prepared in a known manner, e.g. by extending the active ingredient with solvents and/or carriers, if desired using emulsifiers and dispersants. Solvents/auxiliaries, which are suitable, are essentially:
water, aromatic solvents (for example Solvesso products, xylene), paraffins (for example mineral fractions), alcohols (for example methanol, butanol, pentanol, benzyl alcohol), ketones (for example cyclohexanone, gammabutyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

The following are examples of formulations: 1. Products for dilution with water

### A Soluble Concentrates (SL)

10 parts by weight of a compound according to the invention are dissolved in water or in a water-soluble solvent. As an alternative, wetters or other auxiliaries are added. The active ingredient dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

### E Suspensions (SC, OD)

In an agitated ball mill, 20 parts by weight of a compound according to the invention are milled with addition of dispersant, wetters and water or an organic solvent to give a fine active ingredient suspension. Dilution with water gives a stable suspension of the active ingredient.

### F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active ingredient.

### G Water-Dispersible Powders and Water-Soluble Powders (WP, SP)

75 parts by weight of a compound according to the invention are ground in a rotorstator mill with addition of dispersant, wetters and silica gel. Dilution with water gives a stable dispersion or solution with the active ingredient.

### 2. Products to be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 parts by weight of a compound according to the invention is ground finely and associated with 95.5% carriers. Current methods are extrusion, spray drying or the fluidized bed. This gives granules to be applied undiluted.

J ULV solutions (UL)

10 parts by weight of a compound according to the invention are dissolved in an organic solvent, for example xylene. This gives a product to be applied undiluted.

The active ingredients can be used as such, in the form of their formulations or the use forms prepared therefrom, eg. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

Compositions which can be used according to the invention may also contain other active ingredients, for example other pesticides such as insecticides and herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents usually are admixed with the agents according to the invention in a weight ratio of 1:100 to 100:1.

The following list of pesticides together with which the compounds Ia and lib and their salts according to the invention can be used, is intended to illustrate the possible combinations, but not to impose any limitation:

Organo(thio)phosphates: acephate, azamethiphos, azinphos-ethyl, azinphos-methyl, cadusafos, chlorethoxyfos, chlorfenvinphos, chlormephos, chlorpyrifos, chlorpyrifosmethyl, chlorfenvinphos, coumaphos, cyanophos, demeton-5-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, isoxathion, malathion, mecarbam, methamidophos, methidathion, methyl-parathion, mevinphos, monocrotophos, naled, omethoate, oxydemeton-methyl, paraoxon, parathion, parathion-methyl, phenthoate, phorate, phosalone, phosmet, phosphamidon, phorate, phoxim, pirimiphos, pirimiphos-methyl, profenofos, propetamphos, prothiofos, pyraclofos, pyridaphenthion, quinalphos, sulfotep, sulprophos, tebupirimfos, temephos, terbufos, tetrachlorvinphos, thiometon, triazophos, trichlorfon, vamidothion;

Carbamates: alanycarb, aldicarb, bendiocarb, benfuracarb, butocarboxim, butoxycarboxim, carbaryl, carbofuran, carbosulfan, ethiofoncarb, fenobucarb, fenoxycarb, formethanat, furathiocarb, isoprocarb, methiocarb, methomyl, metolcarb, oxamyl, pirimicarb, propoxur, thiodicarb, thiofanox, triazemate, trimethacarb, XMC, xylylcarb;

Pyrethroids: acrinathrin, allethrin, d-cis-trans allethrin, d-trans allethrin, bifenthrin, bioallethrin, bioallethrin S-cyclopentenyl, bioresmethrin, cycloprothrin, cyfluthrin, betacyfluthrin, cyhalothrin, lambda-cyhalothrin, gamma-cyhalothrin, cyphenothrin, cypermethrin, alpha-cypermethrin, beta-cypermethrin, theta-cypermethrin, zetacypermethrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, profluthrin, pyrethrin I and II, resmethrin, RU 15525, silafluofen, tau-fluvalinate, tefluthrin, tetramethrin, tralomethrin, transfluthrin, dimefluthrin, ZXI 8901;

Growth regulators: a) chitin synthesis inhibitors: benzoylureas; bistrifluoron, chlorfluazuron, diflubenzuron, flucycloxuron, flufenoxuron, hexaflumuron, lufenuron, novaluron, noviflumuron, teflubenzuron, triflumuron; buprofezin, diofenolan, hexythiazox, etoxazole, clofentezine; b) ecdysone antagonists: chlormafenozide, halofenozide, methoxyfenozide, tebufenozide, azadirachtin; c) juvenoids: pyriproxyfen, hydroprene, kinoprene, methoprene, fenoxycarb; d) lipid biosynthesis inhibitors: spirodiclofen, spiromesifen, spirotetramat;

Nicotinic receptor agonists/antagonists compounds: acetamiprid, clothianidin, dinotefuran, imidacloprid, nitenpyram, thiacloprid, thiamethoxam, nicotine, bensultap, cartap hydrochloride, thiocyclam, thiosultap-sodium; the thiazol compound of formula (Γ¹)

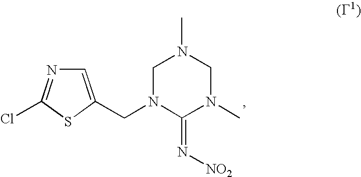

GABA antagonist compounds: acetoprole, chlordane, endosulfan, ethiprole, gammaHCH (lindane), fipronil, vaniliprole, pyrafluprole, pyriprole, vaniliprole, the phenylpyrazole compound of formula Γ²

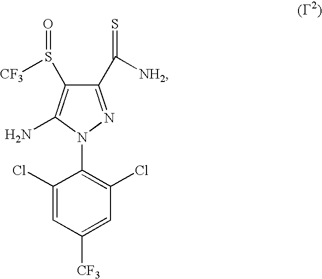

Macrocyclic lactone insecticides: abamectin, emamectin, emamectin benzoate, milbemectin, lepimectin, spinosad.

METI I compounds: fenazaquin, fenpyroximate, flufenerim, pyridaben, pyrimidifen, rotenone, tebufenpyrad, tolfenpyrad;

METI II and III compounds: acequinocyl, fluacryprim, hydramethylnon;

Uncoupler compounds: chlorfenapyr, DNOC;

Oxidative phosphorylation inhibitor compounds: azocyclotin, cyhexatin, diafenthiuron, fenbutatin oxide, propargite, tetradifon;

Moulting disruptor compounds: cyromazine;

Mixed Function Oxidase inhibitor compounds: piperonyl butoxide;

Sodium channel blocker compounds: indoxacarb, metaflumizone, Inorganic compounds: aluminium phosphide, borax, cryolite, cyanide, sulfuryl fluoride, phosphine;

Microbial disruptors of insect midgut membranes: bacillus thuringiensis subsp. israel-ensis, bacillus sphaericus, bacillus thuringiensis subsp. aizawai, bacillus thuringiensis subsp. kurstaki, bacillus thuringiensis subsp. tenebrionis;

Various: amitraz, benclothiaz, benzoximat, bifenazate, bromopropylate, cartap, chinomethionat, chloropicrin, flonicamid, methyl bromide, pyridalyl, pymetrozine, rynaxypursulfur, tartar emetic, thiocyclam, tribufosflubendiamide, cyenopyrafen, flupyrazofos, cyflumetofen, amidoflumet, NNI-0101,

N—R′-2,2-dihalo-1-R″cyclo-propanecarboxamide-2-(2,6-dichloro-α,α,α-tri-fluoro-ptolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, anthranilamide compounds of formula Γ⁵

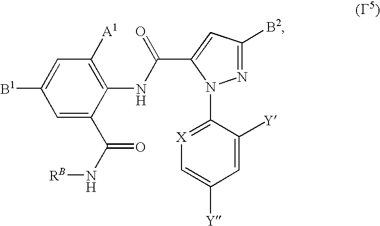

wherein A¹ is CH₃, Cl, Br, I, X is C—H, C—Cl, C—F or N, Y¹ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂, and malononitrile compounds as described in JP 2002 284608, WO 02/89579, WO 02/90320, WO 02/90321, WO 04/06677, WO 04/20399, or JP 2004 99597.

The commercially available compounds of the group A may be found in The Pesticide Manual, 13th Edition, British Crop Protection Council (2003) among other publications. Thiamides of formula Γ² and their preparation have been described in WO 98/28279. Lepimection is known from Agro Project, PJB Publications Ltd, November 2004. Benclothiaz and its preparation have been described in EP-A1454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180.

Anthranilamide compounds of formula Γ⁵ and their preparation have been described in WO 01/70671; WO 02/48137; WO 03/24222, WO 03/15518, WO 04/67528; WO 04/33468; and WO 05/118552.

The aforementioned compositions are particularly useful for protecting plants against infestation of said pests or to combat these pests in infested plants.

However, the compounds of formulae Ia and Ib and their salts are also suitable for the treatment of seeds. Application to the seeds is carried out before sowing, either directly on the seeds or after having pregerminated the latter.

Compositions which are useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)
H Dustable powders (DP, DS)

Preferred FS formulations of compounds of formulae Ia or Ib for seed treatment usually comprise from 0.5 to 80% of the active ingredient, from 0.05 to 5% of a wetter, from 0.5 to 15% of a dispersing agent, from 0.1 to 5% of a thickener, from 5 to 20% of an anti-freeze agent, from 0.1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

For use against ants, termites, wasps, flies, mosquitos, crickets, or cockroaches, compounds of formula I or II, their pyridine N-oxides or their salts are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formulae Ia or Ib as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formulae Ia or Ib, their salts and their respective compositions can also be used in mosquito and fumigating coils, smoke cartridges, vaporizer plates or long-term vaporizers and also in moth papers, moth pads or other heat-independent vaporizer systems.

The compounds of formulae Ia or Ib, their salts, and their compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of compounds of formulae I or II or with an pyridine N-oxide thereof or with a salt thereof or with a composition, containing a pesticidally effective amount of a compound of formula I or II, or a pyridine N-oxide or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formulae Ia or Ib and their salts may be also used to protect growing plants from attack or infestation by pests by contacting the plant with a pesticidally effective amount of compounds of formula Ia or Ib and their salts. As such, “contacting” includes both direct contact (applying the compounds/compositions directly on the pest and/or plant—typically to the foliage, stem or roots of the plant) and indirect contact (applying the compounds/compositions to the locus of the pest and/or plant).

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

The present invention is now illustrated in further detail by the following examples.

The compounds of the invention as well as intermediates were characterized by coupled High Performance Liquid Chromatography/mass spectroscopy (HPLC/MS), by NMR or by their melting points. HPLC column: RP-18 column (Chromolith Speed ROD from Merck KgaA, Germany). Elution: acetonitrile+0.1% trifluoroacetic acid (TFA)/water+0.1% TFA in a ratio of from 5:95 to 95:5 in 5 minutes at 40° C. MS Quadrupol electrospray ionisation, 80 V (positive modus).

### EXAMPLE A

### Preparation of N-(2,3-Dichloro-phenyl)-N′-(4,5-dihydro-thiazol-2-yl)-N-(3-methyl-benzyl)-hydrazine

### B.1 N-(2,3-Dichloro-phenyl)-N-(3-methyl-benzyl)-hydrazine hydrochloride

Sodium amide (0.41 g, 10.50 mmol) was suspended in tetrahydrofurane (15 mL) and cooled to 5 to 8° C. A solution of 2,3-Dichloro-phenyl hydrazine (1.77 g, 10.00 mmol) in tetrahydrofurane (20 mL) was added dropwise and the reaction mixture was allowed to warm to room temperature. Then N2 was bubbled through the mixture for 2 h. At a temperature of 10 to 15° C. 3-methyl benzylbromide (2.04 g, 11.00 mmol) was added dropwise and the mixture was stirred over night at ambient temperature. The mixture was poured into water (25 mL) and the tetrahydrofurane was removed in vacuo. The aqueous layer was extracted with ethyl acetate (4 times), washed with water (2 times) and dried over MgSO₄. The organic phase was treated with ethereal hydrochloride (2 M in diethyl ether), the precipitate was seperated by filtration and dried in vacuo. Thus, N-(2,3-dichloro-phenyl)-N-(3-methyl-benzyl)-hydrazine hydrochloride (2.28 g, 7.18 mmol, 72%) was isolated as a white solid.

### A.2 N-(2,3-Dichloro-phenyl)-N′-(4,5-dihydro-thiazol-2-yl)-N-(3-methyl-benzyl)hydrazine

A solution of N-(2,3-dichloro-phenyl)-N-(3-methyl-benzyl)-hydrazine (0.46 g, 1.64 mmol) in methyl-tert-butyl ether (15 mL) was obtained from the extraction of a solution of the hydrochloride prepared as described above with an aqueous NaOH solution. 2-Bromoethylisothiocyanate (0.27 g, 1.64 mmol) was added dropwise and the reaction mixture was stirred over night. The precipitate was seperated by filtration, dissolved in water and treated with an aqueous NaOH solution (1 M). The aqueous solution was extracted with methylene chloride (3 times), the combined organic layers were washed with an aqueous NaOH solution (1 M) and dried over MgSO₄. Evaporation of the solvent yielded N-(2,3-Dichloro-phenyl)-N′-(4,5-dihydro-thiazol-2-yl)-N-(3-methyl-benzyl)-hydrazine (0.30 g, 0.82 mmol, 50%) as a white solid.

### EXAMPLE B

### Preparation of N′-(4,5-Dihydro-thiazol-2-yl)-N-(2,3-dimethyl-phenyl)-N-(3-methyl-but-2-enyl)-hydrazine

### B.1 N-(4,5-Dihydro-thiazol-2-yl)-N′-(2,3-dimethyl-phenyl)-hydrazine

2,3-Dimethyl-phenyl-hydrazine (10.90 g, 80.0 mmol) was dissolved in diethyl ether (400 mL) and cooled to 0-5° C. 2-Chloroethylisothiocyanate (9.73 g, 80.0 mmol) was added dropwise, the reaction mixture allowed to warm to room temperature and stirred for 3 h. An aqueous solution of NaOH (1 M, 80 mL) was added and the mixture stirred for 30 min. After dilution with ethyl acetate (100 mL), the organic layer was separated. The aqueous layer was extracted with ethyl acetate twice and the combined organic layers were washed with water twice and dried over Na₂SO₄. After removal of the solvents in vacuo, the residue was triturated with a mixture of CH₂Cl₂ (100 mL) and petrol ether (400 mL). Filtration and drying yielded N-(4,5-Dihydro-thiazol-2-yl)-N′-(2,3-dimethyl-phenyl)hydrazine (12.01 g, 54.3 mmol, 68%).

### B.2 N-(4,5-Dihydro-thiazol-2-yl)-N′-(2,3-dimethyl-phenyl)-hydrazinecarboxylic acid tert-butyl ester

N-(4,5-Dihydro-thiazol-2-yl)-N′-(2,3-dimethyl-phenyl)-hydrazine (6.90 g, 31.18 mmol) was dissolved in tetrahydrofurane (120 mL) and cooled to 0° C. Pyridine (3.70 g, 46.76 mmol), dimethylaminopyridine (DMAP, 0.10 g, 0.82 mmol) and a solution of Di-tert-butyl dicarbonate (10.21 g, 46.76 mmol) in tetrahydrofurane (80 mL) were added successively. The reaction mixture was stirred at 0° C. for 2 h and stirring was continued over night at room temperature. The mixture was concentrated by half and treated with water (200 mL) and NaHCO₃ to obtain a pH value of about 8. Extraction with CH₂Cl₂ (3 times), washing the combined organic layers with water (twice) and drying over Na₂SO₄ yielded the crude material that was purified by column chromatography (SiO₂, cyclohexane/ethyl acetate 1:0→6:4) to give N-(4,5-Dihydro-thiazol-2-yl)-N′-(2,3-dimethyl-phenyl)-hydrazinecarboxylic acid tert-butyl ester (5.40 g, 16.80 mmol, 54%) and as a sideproduct 2-[(2,3-Dimethyl-phenyl)-hydrazono]-thiazolidine-3-carboxylic acid tert-butyl ester (1.00 g, 3.11 mmol, 10%).

### B.3 N-(4,5-Dihydro-thiazol-2-yl)-N′-(2,3-dimethyl-phenyl)-N′-(3-methyl-but-2-enyl)hydrazine

N-(4,5-Dihydro-thiazol-2-yl)-N′-(2,3-dimethyl-phenyl)-hydrazinecarboxylic acid tert-butyl ester (0.48 g, 1.50 mmol) was dissolved in tetrahydrofurane (10 mL) and cooled to 0° C. A solution of lithium hexamethyldisilazane (LHMDS,1 M, 1.65 mL, 1.65 mmol) was added dropwise and the reaction mixture was stirred for 30 min. Then a solution of 3,3-Dimethylallylbromide (0.40 g, 2.70 mmol) in tetrahydrofurane (5 mL) was added, the mixture warmed to room temperature and stirred for 6 h. The reaction mixture was quenched with NH₄Cl solution, extracted with ethyl acetate (2 times), the combined organic phases washed with NaCl solution and dried over Na₂SO₄. The residue (0.22 g, 0.56 mmol) was dissolved in CH₂Cl₂ (15 mL) and treated with trifluoroacetic acid (TFA, 1.49 g, 13.07 mmol). The reaction mixture was stirred for 20 h. Evaporation of the solvent yielded N′-(4,5-Dihydro-thiazol-2-yl)-N-(2,3-dimethyl-phenyl)-N-(3-methyl-but-2-enyl)-hydrazine as its trifluoroacetate-salt (0.21 g, 0.52 mmol, 92%). The salt was dissolved in CH₂Cl₂ (15 mL) and the solution was extracted with a mixture of an aqueous solution of NaOH (2 M, 1.25 mL) and water (15 mL). The organic phase was washed with water (twice) and an aqueous solution of NaCl succesively and dried over Na₂SO₄. Evaporation of the solvent yielded N′-(4,5-Dihydrothiazol-2-yl)-N-(2,3-dimethyl-phenyl)-N-(3-methyl-but-2-enyl)-hydrazine (0.07 g, 0.24 mmol, 43%).

### EXAMPLE C

### Preparation of N′-(4-Chloro-3-trifluoromethyl-benzyl)-N-(4,5-dihydro-thiazol-2-yl)-N′-phenyl-hydrazinecarboxylic acid tert-butyl ester

N-(4,5-Dihydro-thiazol-2-yl)-N′-(phenyl)-hydrazinecarboxylic acid tert-butyl ester (prepared according to example 2.2, 0.42 g, 1.30 mmol) was dissolved in tetrahydrofurane (10 mL) and cooled to −30° C. A solution of lithium hexamethyldisilazane (LHMDS, 1 M, 1.43 mL, 1.43 mmol) was added dropwise and the reaction mixture was stirred for 30 min. Then a solution of 4-Chloro-3-(trifluoromethyl)benzyl bromide (0.47 g, 1.71 mmol) in tetrahydrofurane (2 mL) was added and the mixture warmed to room temperature over night. The volatiles were evaporated, the residue charged on celite and purified by column chromatography (SiO₂, cyclohexane/ethyl acetate) to yield N′-(4-Chloro-3-trifluoromethyl-benzyl)-N-(4,5-dihydro-thiazol-2-yl)-N′-phenyl hydrazinecarboxylic acid tert-butyl ester (0.34 g, 0.66 mmol, 51%).

### EXAMPLE D

### Preparation of N-(4-Chloro-3-trifluoromethyl-benzyl)-N′-(4,5-dihydro-thiazol-2-yl)-N-phenyl-hydrazine

N′-(4-Chloro-3-trifluoromethyl-benzyl)-N-(4,5-dihydro-thiazol-2-yl)-N′-phenyl hydrazinecarboxylic acid tert-butyl ester (0.33 g, 0.64 mmol) was dissolved in CH₂Cl₂ (10 mL) and treated with trifluoroacetic acid (TFA, 0.73 g, 6.42 mmol). The reaction mixture was stirred for 20 h and quenched with 5% aqueous K₂CO₃ solution. The organic phase was separated, washed with water (2 times) and dried over Na₂SO₄. The residue was triturated with diethyl ether to yield N-(4-Chloro-3-trifluoromethyl-benzyl)-N′-(4,5-dihydro-thiazol-2-yl)-N-phenyl-hydrazine (75 mg, 0.18 mmol, 28%).

The compounds of the general formulae Ia-B and Ib-B (examples 1 to 64) can be prepared accordingly. The physico-chemical data of these compounds are listed in table 37.

TABLE 37

| | |
|---|---|
| | (Ia-B) |
| 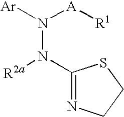 | |
| | (Ib-B) |
| 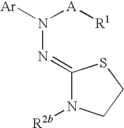 | |

| | | | | | | Physico-chemical |
|---|---|---|---|---|---|---|
| | | | | | | data (m.p. [° C.]); |
| | | | | | | HPLC/MS |
| Ex. | Form. | Ar | R¹ | A | R^{2a}/R^{2b} | min/[M + H]⁺ |
| 1 | Ia-B | Ar.0 | Ar.0 | CH₂ | H | 127-129 |
| 2 | Ia-B | Ar.19 | Ar.8 | CH₂ | H | 183-185 |
| 3 | Ia-B | Ar.19 | Ar.9 | CH₂ | H | 155-158 |
| 4 | Ia-B | Ar.19 | Ar.79 | CH₂ | H | 156-157 |
| 5 | Ia-B | Ar.18 | Ar.79 | CH₂ | H | ¹H-NMR* |
| 6 | Ia-B | Ar.19 | Ar.78 | CH₂ | H | 93-95 |
| 7 | Ia-B | Ar.18 | Ar.9 | CH₂ | H | 123-127 |
| 8 | Ia-B | Ar.19 | Ar.24 | CH₂ | H | 3.25, 340 |
| 9 | Ia-B | Ar.0 | CH₂Cl | C(O) | H | 1.64, 270 |
| 10 | Ia-B | Ar.19 | Ar.74 | CH₂ | H | 172-175 |
| 11 | Ia-B | Ar.19 | Ar.73 | CH₂ | H | 162-167 |
| 12 | Ia-B | Ar.19 | 3-CN-Ph | CH₂ | H | 200-202 |
| 13 | Ia-B | Ar.19 | Ar.69 | CH₂ | H | 130-140 |
| 14 | Ia-B | Ar.9 | CH₃ | CH₂ | H | 2.14, 236 |
| 15 | Ia-B | Ar.0 | NHPh | C(O) | H | 2.00, 313 |
| 16 | Ia-B | Ar.0 | CH₃ | CH₂ | H | 1.79, 222 |
| 17 | Ia-B | Ar.0 | (CH₂)₂CH₃ | CH₂ | H | 2.34, 250 |
| 18 | Ia-B | Ar.4 | CH₃ | CH₂ | H | 2.17, 236 |
| 19 | Ia-B | Ar.14 | CH₃ | CH₂ | H | 2.07, 236 |
| 20 | Ia-B | Ar.0 | NHCH₂C(O)OEt | C(O) | H | 2.73, 323 |
| 21 | Ia-B | Ar.19 | Ar.80 | CH₂ | H | 125-132 |
| 22 | Ia-B | Ar.2 | Ar.79 | CH₂ | H | 3.02, 391 |
| 23 | Ia-B | Ar.19 | CH═CH₂ | CH₂ | H | 2.44, 262 |
| 24 | Ia-B | Ar.19 | OC(CH₃)₃ | C(O) | CH₃ | 2.60, 336 |
| 25 | Ia-B | Ar.19 | (CH₂)₃ | | | 2.29, 262 |
| 26 | Ib-B | Ar.19 | CH═C(CH₃)₂ | CH₂ | C(O)OC(CH₃)₃ | 3.06, 390 |
| 27 | Ib-B | Ar.19 | OC(CH₃)₃ | C(O) | CH₂—C≡H | 106-108 |
| 28 | Ib-B | Ar.19 | OC(CH₃)₃ | C(O) | 3,5-F₂-phenyl-CH₂ | 131-134 |
| 29 | Ia-B | Ar.19 | OC(CH₃)₃ | C(O) | C₂H₅ | 2.71, 350 |
| 30 | Ia-B | Ar.19 | CH═C(CH₃)₂ | CH₂ | H | 2.64, 290 |
| 31 | Ib-B | Ar.19 | OC(CH₃)₃ | C(O) | 3,5-(CH₃O)₂—Ph—CH₂ | 3.29, 472 |
| 32 | Ib-B | Ar.19 | OC(CH₃)₃ | C(O) | 3-F,5-CF₃—Ph—CH₂ | 161-163 |
| 33 | Ia-B | Ar.19 | 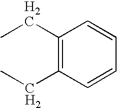 | | | 138-141 |
| 34 | Ia-B | Ar.18 | Ar.79 | CH₂ | H | 124-126 |
| | | | | | | as hydrochloride |
| 35 | Ia-B | Ar.19 | CH═C(CH₃)₂ | CH₂ | H | as trifluoro- |
| | | | | | | acetate salt |
| 36 | Ia-B | Ar.19 | OC(CH₃)₃ | C(O) | CH₂—OCH₃ | 2.91, 366 |
| 37 | Ib-B | Ar.19 | OC(CH₃)₃ | C(O) | SO₂-Ar.79 | 136-139 |
| 38 | Ia-B | Ar.19 | OC(CH₃)₃ | C(O) | C(O)OCH₃ | 104-108 |
| 39 | Ib-B | Ar.19 | OC(CH₃)₃ | C(O) | CH₂C(O)OCH₃ | 2.97, 394 |
| 40 | Ia-B | Ar.19 | OC(CH₃)₃ | C(O) | C(O)—CH₂OCH₃ | 3.54, 394 |
| 41 | Ib-B | Ar.19 | OC(CH₃)₃ | C(O) | CH₂—CN | 383 [M + Na]⁺ |
| 42 | Ia-B | Ar.19 | OC(CH₃)₃ | C(O) | C(O)-Ar.79 | 4.33, 454 |
| 43 | Ib-B | Ar.19 | OC(CH₃)₃ | C(O) | CH₂-Ar.6 | 3.37, 430 |
| 44 | Ib-B | Ar.19 | OC(CH₃)₃ | C(O) | C(O)OC(CH₃)₃ | 3.72, 422 |
| 45 | Ib-B | Ar.19 | OC(CH₃)₃ | C(O) | CH₂-Ar.79 | 107-112 |
| 46 | Ib-B | Ar.19 | OC(CH₃)₃ | C(O) | CH₂-Ar.80 | 122-130 |
| 47 | Ib-B | Ar.19 | Ar.79 | C(O) | CH₂-Ar.79 | 154-158 |
| 48 | Ib-B | Ar.19 | CH₃ | C(O) | CH₂-Ar.79 | 3.35, 382 |
| 49 | Ib-B | Ar.19 | OC(CH₃)₃ | C(O) | CH₂-Ar.14 | 3.30, 426 |
| 50 | Ib-B | Ar.19 | OC(CH₃)₃ | C(O) | CH₂-Ar.15 | 3.60, 480 |
| 51 | Ib-B | Ar.19 | OC(CH₃)₃ | C(O) | CH₂-(4-NO₂-Ph) | 3.37, 457 |
| 52 | Ib-B | Ar.19 | OC(CH₃)₃ | C(O) | CH₂-Ar.1 | 3.24, 430 |
| 53 | Ia-B | Ar.19 | Ar.89 | CH₂ | C(O)OC(CH₃)₃ | 3.54, 414 |
| | | | | | | [M − 101]⁺ |
| 54 | Ia-B | Ar.19 | Ar.77 | CH₂ | C(O)OC(CH₃)₃ | 3.68, 470 |
| | | | | | | [M − 101]⁺ |
| 55 | Ia-B | Ar.19 | Ar.77 | CH₂ | H | 3.28, 470 |
| 56 | Ia-B | Ar.19 | Ar.73 | CH₂ | C(O)OC(CH₃)₃ | 3.60, 480 |
| 57 | Ia-B | Ar.19 | Ar.89 | CH₂ | H | 169-172 |
| 58 | Ia-B | Ar.19 | Ar.73 | CH₂ | H | 161-165 |
| 59 | Ib-B | Ar.19 | OC(CH₃)₃ | C(O) | CH₂-Ar.3 | 3.33, 446 |
| 60 | Ib-B | Ar.19 | OC(CH₃)₃ | C(O) | CH₂-Ar.4 | 3.25, 426 |
| 61 | Ib-B | Ar.19 | OC(CH₃)₃ | C(O) | CH₂-(3-I-Ph) | 3.52, 538 |
| 62 | Ia-B | Ar.82 | N(CH₃)₂ | C(O) | H | 2.00, 317 |
| 63 | Ib-B | Ar.19 | Ar.79 | CH₂ | C(O)CH₃ | 4.05, 382 |
| 64 | Ib-B | Ar.19 | Ar.79 | CH₂ | C(O)OC(CH₃)₃ | 97-100 |

In table 25 Ph stands forphenyl, Et means ethyl.

### 2. Examples of Action Against Pests

The action of the compounds of the formulae Ia and Ib against pests was demonstrated by the following experiments:

2.1 Activity against Vetch Aphid (Megoura viciae)

The active compounds were formulated in DMSO/water (1:3). Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

In this test, compounds 2, 3, 4, 5, 6, 7, 8, 9, 10, 11, 12, 13, 17, 22, 25, 30, 34, 36, 40, 42, 44, 54, 55, 57, 58 and 63 at 2500 ppm showed over 75% mortality compared to 0% mortality of untreated controls.

2.2 Activity against Cotton Aphid (Aphis gossypii)

The active compounds were formulated in acetone/water (1:1) and 100 ppm Kinetic™ surfactant. Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds 1, 2, 3, 4, 5, 6, 7, 8, 9, 10, 11, 12, 13, 16, 17, 18, 21, 22, 23, 34, 55, 57, 58, 59, 63 and 64 at 300 ppm showed over 50% mortality.

2.3. Activity against Green Peach Aphid (Myzus persicae)

Pepper plants in the 2^{nd} leaf-pair stage (variety ‘California Wonder’) are infested with approximately 40 laboratory-reared aphids by placing infested leaf sections on top of the test plants. The leaf sections are removed after 24 hours. The leaves of the intact plants are dipped into gradient solutions of the test compound. Aphid mortality on the treated plants, relative to mortality on check plants, is determined after 5 days.

In this test, compound of example no. 1, 2, 3, 4, 5, 6, 7, 8, 9, 10, 13, 18, 21, 23, 34, 55, 57, 58, 59 and 63 at 300 ppm showed over 50% mortality in comparison with untreated controls.

2.4 Activity against Brown Planthopper (nilaparvata lugens)

The active compounds were formulated as a 20:80 acetone:water solution. Surfactant (Alkamuls EL 620) was added at the rate of 0.1% (vol/vol).

Potted rice plants of 3-4 weeks of age are sprayed with 10 ml of the test solution using air driven hand atomizer (Devillbis atomizer) at 1.7 bar. The treated plants are allowed to dry for about 1 hour and covered with Mylar cages. The plants are inoculated with 10 adults of the specie (5 male and 5 females) and kept at 25-27° C. and 50-60% humidity for 3 days. Mortality is assed after 24, 48 and 72 hours after treatment. Dead insects are usually found in the water surface. Each treatment is replicated once.

In this test, compounds of examples no. 3, 4, 6, 7, 8, 10 and 58 at 300 ppm showed a mortality of at least 50% in comparison with untreated controls.

1-25. (canceled)

26. Azoline compounds of the general formulae Ia or Ib, 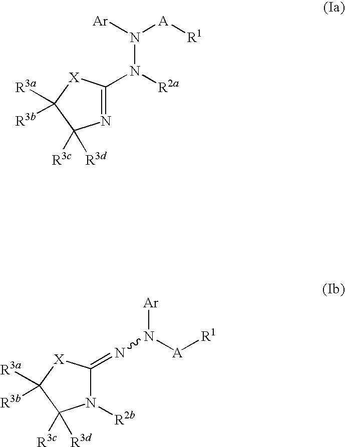
wherein
X is sulfur, oxygen or a radical NR⁴;
Ar is an aromatic radical selected from the group consisting of phenyl and a 5 or 6 membered heteroaromatic ring, which contains 1, 2, 3 or 4 heteroatoms selected from oxygen, sulfur and nitrogen as ring members, wherein Ar is unsubstituted or may carry any combination of 1, 2, 3, 4 or 5 radicals R^{y1};
R¹ is selected from the group consisting of CN, SH, OH,
C₁-C₆-alkyl, which carries 1, 2 or 3 radicals R^{a1},
C₃-C₁₀-cycloalkyl, which may be unsubstituted or may carry 1, 2, 3, 4 or 5 radicals R^{b1},
C₂-C₆-alkenyl, which may carry 1, 2 or 3 radicals R″,
C₂-C₆-alkynyl, which may carry 1, 2 or 3 radicals R″, a radical O-R^{z1},
a radical S(O)ₘ—R^{Z} with m being 0, 1 or 2,
a radical C(O)—R^{z3},
a radical NR^{z4}R^{z5},
phenyl, which is unsubstituted or may carry any combination of 1, 2, 3, 4 or 5 radicals R^{y2},
and a 5 or 6 membered heteroaromatic ring, which contains 1, 2, 3 or 4 heteroatoms selected from oxygen, sulfur and nitrogen as ring members,
and which is unsubstituted or may carry any combination of 1, 2, 3, or 4 radicals R^{y3};
R^{2a}, R^{2b} are each independently selected from the group consisting of hydrogen, formyl, CN, C₁-C₆-alkyl, C₃-C₆-alkenyl, C₃-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, C₁-C₆-alkylsulfonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzyl, phenoxycarbonyl, phenylsulfonyl, 5 or 6 membered hetarylmethyl, 5 or 6 membered hetarylcarbonyl and benzoyl each of the last seven mentioned radicals may be unsubstituted or may carry any combination of 1, 2, 3, 4 or 5 radicals R^{b2}, and wherein the 5 or 6 membered heteroaromatic ring in hetarylmethyl and hetarylcarbonyl contains 1, 2, 3 or 4 heteroatoms selected from oxygen, sulfur and nitrogen as ring members;
R¹ together with R^{2a} may also form a linear C₂-C₄-alkandiyl, which may carry 1, 2, 3 or 4 radicals R^{d} independently selected from halogen, C₁-C₄-alkyl and C₁-C₄-haloalkyl, wherein the radicals which are bound to adjacent carbon atoms of alkylene may together with the carbon atoms to which they are bound, form a fused phenyl ring, which may carry 1, 2, 3 or 4 radicals independently selected from halogen, C₁-C₄-alkyl and C₁-C₄-haloalkyl, and wherein 1 or 2 non-adjacent CH₂-moieties of C₂-C₄-alkandiyl may be replaced by oxygen or a radical N-R^{q}; wherein R^{q} has one of the meanings given for R^{2a};
R^{3a}, R^{3b}, R^{3c}, R^{3d} are each independently selected from the group consisting of hydrogen, halogen, cyano, nitro, hydroxy, mercapto, amino, C₁-C₆-haloalkyl, C₁-C₆-alkyl, C₁-C₆-alkylamino, Di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl or benzyl, each of the last two mentioned radicals may be unsubstituted or may carry any combination of 1, 2, 3, 4 or 5 radicals R^{b3};
R⁴ is selected from the group consisting of hydrogen, formyl, CN, C₁-C₆-alkyl, C₃-C₆-alkenyl, C₃-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a4},
C(O)NR^{a}R^{b}, (SO₂)NR^{a}R^{b},
phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b4}, and wherein the 5 or 6 membered heteroaromatic ring in hetarylmethyl and hetarylcarbonyl contains 1, 2, 3 or 4 heteroatoms selected from oxygen, sulfur and nitrogen as ring members;
A is linear C₁-C₄-alkandiyl, which is unsubstituted or may carry any combination of 1, 2, 3 or 4 radicals R^{x}; or A can also be C(O) when R¹ is a radical O—R^{z1} or NR^{z4}R^{z5};
R^{x} is selected from the group consisting of halogen, C₁-C₆-alkyl and C₁-C₆-haloalkyl, wherein the carbon atoms in these groups may carry any combination of 1, 2 or 3 radicals R^{ax};
R^{y1}, R^{y2}, R^{y3} independently of each other are selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{c}R^{d}, formyl, C₁-C₆-alkylcarbonyl,
C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{ay},
C(O)NR^{a}R^{b}, (SO₂)NR^{a}R^{b}, and radicals of the formula Y-Cy, wherein
Y is a single bond, oxygen, sulfur or C₁-C₆-alkandiyl, wherein one carbon might be replaced with oxygen.
Cy is selected from the group consisting of C₃-C₁₂-cycloalkyl, which is unsubstituted or substituted with any combination of 1, 2, 3, 4 or 5 radicals R^{bx}, phenyl, naphthyl and mono- or bicyclic 5- to 10-membered heterocyclyl, which contains 1, 2, 3 or 4 heteroatoms selected from oxygen, sulfur and nitrogen as ring members, wherein Cy is unsubstituted or may carry any combination of 1, 2, 3, 4 or 5 radicals R^{by};
and wherein two radicals R^{y1}, R^{y2} or R^{y3} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from oxygen, sulfur and nitrogen as ring members, and wherein the fused ring is unsubstituted or may carry any combination of 1, 2, 3, or 4 radicals R^{by};
R^{z1} is selected from the group consisting of C₁-C₆-alkyl, C₁-C₆-haloalkyl, C₁-C₆-alkoxy-C₁-C₄-alkyl, C₁-C₆-alkylcarbonyl, C₁-C₆-haloalkylcarbonyl, C₂-C₆-alkenylcarbonyl, C₂-C₆-alkynylcarbonyl, phenyl, benzyl and benzoyl, each of the last three mentioned radicals may be unsubstituted or may carry any combination of 1, 2, 3, 4 or 5 radicals R^{bz};
R^{z2} is selected from the group consisting of C₁-C₆-alkyl, C₁-C₆-haloalkyl, and phenyl, which may be unsubstituted or may carry any combination of 1, 2, 3, 4 or 5 radicals R^{bz};
R^{z3} is selected from the group consisting of hydrogen, C₁-C₆-alkyl, C₁-C₆-haloalkyl, C₁-C₆-alkoxy-C₁-C₄-alkyl, C₁-C₆-alkoxy, C₁-C₆-haloalkoxy, C₂-C₆-alkenyl, C₂-C₆-alkynyl, C₂-C₆-alkenyloxy, C₂-C₆-alkynyloxy, a radical NR^{c}R^{d},
phenyl, benzyl and phenoxy, each of the last three mentioned radicals may be unsubstituted or may carry any combination of 1, 2, 3, 4 or 5 radicals R^{bz};
R^{z4}, R^{z5} are each independently selected from the group consisting of hydrogen, C₁-C₆-alkyl, C₂-C₆-alkenyl, C₂-C₆-alkynyl, C₂-C₆-alkylcarbonyl, C₂-C₆-alkoxycarbonyl, wherein the carbon atoms in these groups may carry any combination of 1, 2 or 3 radicals R^{az}, or phenyl, phenyl-C₁-C₄-alkyl, benzoyl, wherein the last three mentioned groups may carry any combination of 1, 2, 3, 4 or 5 radicals R^{bz}
R^{a}, R^{b} are each independently selected from the group consisting of hydrogen, C₁-C₆-alkyl, C₂-C₆-alkenyl, or C₂-C₆-alkynyl, wherein the carbon atoms in these groups may carry any combination of 1, 2 or 3 radicals R^{ay};
R^{a1}, R^{a2}, R^{a3}, R^{a4}, R^{ax}, R^{ay} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}; R^{b2}, R^{b3}, R^{b4}, R^{bx}, R^{by} and R^{bz} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R^{c}, R^{d} are each independently selected from the group consisting of hydrogen, C₁-C₆-alkyl, C₂-C₆-alkenyl, or C₂-C₆-alkynyl, wherein the carbon atoms in these groups may carry any combination of 1, 2 or 3 radicals R^{ay};
R^{c1} is selected from the group consisting of halogen, OH, C₁-C₆-alkoxy and C₃-C₆-cycloalkyl;
and the salts thereof.

27. The compound of 26, wherein Ar is phenyl, which is unsubstituted or may carry any combination of 1, 2, 3, 4 or 5 radicals R^{y1}.

28. The compound of claim 26, wherein said phenyl carries 1, 2, 3, 4 or 5 radicals R^{y1}, which are, independently of each other selected from the group consisting of halogen, C₁-C₆-alkyl, C₁-C₆-haloalkyl, C₁-C₆-alkylthio, C₁-C₆-haloalkylthio, C₁-C₆-alkoxy, di(C₁-C₆-alkyl)amino and C₁-C₆-haloalkoxy.

29. The compound of claim 26, wherein A is a radical CR⁵R⁶, wherein R⁵ is selected from hydrogen or C₁-C₄-alkyl and R⁶ is selected from hydrogen, halogen, C₁-C₄-alkyl, C₁-C₄-haloalkyl, C₁-C₄-alkoxy-C₁-C₄-alkyl, or phenyl, which is unsubstituted or substituted with any combination of 1, 2, 3, 4 or 5 radicals selected from halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy.

30. The compound of claim 29, wherein A is CH₂.

31. The compound of claim 26, wherein R¹ is phenyl, which is unsubstituted or may carry any combination of 1, 2, 3, 4 or 5 radicals R^{y2}.

32. The compound of claim 31, wherein said phenyl carries 1, 2, 3, 4 or 5 radicals R^{y2}, which are, independently of each other selected from the group consisting of halogen, C₁-C₆-alkyl, C₁-C₆-haloalkyl, C₁-C₆-alkylthio, C₁-C₆-haloalkylthio, C₁-C₆-alkoxy, di(C₁-C₆-alkyl)amino and C₁-C₆-haloalkoxy.

33. The compound of claim 25, wherein R¹ is selected from the group consisting of CN,
C₃-C₁₀-cycloalkyl, which may be unsubstituted or may carry 1, 2, 3, 4 or 5 radicals R^{b1},
C₂-C₆-alkenyl, which may carry 1, 2 or 3 radicals R^{c1},
C₂-C₆-alkynyl, which may carry 1, 2 or 3 radicals R^{c1},
a radical O-R^{z1},
a radical S(O)ₘ—R^{z2} with m being 0, 1 or 2, and
a radical C(O)—R^{z3}.

34. The compound of claim 33, wherein A is CH₂ or CH₂CH₂ and R¹ is selected from the group consisting of CN,
C₂-C₆-alkenyl,
C₂-C₆-alkynyl,
a radical O-R^{z1},
a radical S(O)ₘ—R^{z2} with m being 0, 1 or 2, and a radical C(O)—R^{z3}.

35. The compound of claim 26, wherein R¹ is C₁-C₆-alkyl, which carries 1, 2 or 3 radicals R^{a1}.

36. The compound of claim 26, wherein R^{2a} and R^{2b} are selected from the group consisting of hydrogen, C₁-C₄-alkyl, formyl, CN, C₁-C₆-alkylcarbonyl, C₁-C₄-haloalkylcarbonyl, C₁-C₆-alkoxycarbonyl, C₁-C₄-alkoxy-C₁-C₄-alkoxycarbonyl or C₁-C₆-alkylthiocarbonyl.

37. The compound of claim 26, wherein R^{2a} or R^{2b} are hydrogen.

38. The compound of claim 26, wherein the radicals R^{3a}, R^{3b}, R^{3c} and R^{3d} are each hydrogen.

39. The compound of claim 26, wherein X is S.

40. The compound of claim 26, wherein X is O.

41. The compound of claim 26, wherein X is NR⁴.

42. A composition comprising at least one compound of formulae Ia or Ib and/or a salt thereof as defined in claim 26 and a carrier material.

43. A method for the control of arthropod pests or nematodes, which comprises contacting the insect, acarid or nematode or their food supply, habitat, breeding ground or their locus with at least one compound of the formulae Ia or Ib and/or a salt thereof as defined in claim 26.

44. A method of protecting growing plants from attack or infestation by arthropod pests or nematodes, which comprises applying to the plants, or to the soil or water in which they are growing, at least one compound of formulae Ia or Ib and/or an agriculturally acceptable salt thereof as defined in claim 43.

45. A method of protecting growing plants from attack or infestation by arthropod pests or nematodes, which comprises applying to the plants, or to the soil or water in which they are growing, at least one compound of formulae Ia or Ib and/or an agriculturally acceptable salt thereof as defined in claim 26.

46. The method of claim 45, wherein said at least one compound of formulae Ia or Ib and/or the salt thereof or a composition comprising them is applied in an amount of from 5 g/ha to 2000 g/ha, calculated as the compound of formulae Ia or Ib.

47. A method of protection of seed comprising contacting the seeds with said at least one compound of formulae Ia or Ib and/or an agriculturally acceptable salt thereof as defined in claim 26 or a composition containing at least one of these compounds in pesticidally effective amounts.

48. A method as claimed in claim 47 wherein the said at least one compound of formulae Ia or Ib and/or an agriculturally acceptable salt thereof or said composition comprising at least one of these compounds is applied in an amount of from 0.1 g to 10 kg per 100 kg of seeds.

49. Seed, comprising at least one compound of formulae Ia or Ib and/or an agriculturally acceptable salt thereof as defined in claim 26 in an amount of from 0.1 g to 10 kg per 100 kg of seeds, calculated as the compound of formulae Ia or Ib.

50. A method for treating, controlling, preventing or protecting animals against infestation or infection by parasites which comprises administering or applying to the animals a parasiticidally effective amount of at least one compound of formulae Ia or Ib and/or an veterinarily acceptable salt thereof as defined in claim 26.
